(19) United States
(12) Patent Application Publication
(10) Pub. No.: US 2013/0252951 A1
(43) Pub. Date: Sep. 26, 2013

(54) 7-HYDROXY-PYRAZOLO[1,5-A] PYRIMIDINE COMPOUNDS AND THEIR USE AS CCR2 RECEPTOR ANTAGONISTS

(71) Applicants: Joe William Boyd, London (GB); Paul Meo, London (GB); Michael Higginbottom, London (GB); Iain Simpson, London (GB); David Mountford, London (GB); Edward Daniel Savory, London (GB)

(72) Inventors: Joe William Boyd, London (GB); Paul Meo, London (GB); Michael Higginbottom, London (GB); Iain Simpson, London (GB); David Mountford, London (GB); Edward Daniel Savory, London (GB)

(73) Assignee: Proximagen Limited, London (GB)

(21) Appl. No.: 13/876,220

(22) PCT Filed: Sep. 26, 2011
(86) PCT No.: PCT/EP2011/066697

§ 371 Date: Jun. 11, 2013

(30) Foreign Application Priority Data
Sep. 27, 2010 (GB) 1016221.1
Aug. 15, 2011 (GB) 1113971.4

(51) Int. Cl. C07D 487/04

(52) U.S. Cl. CPC C07D 487/04
USPC 514/233.2; 544/281; 514/259.3; 514/255.05; 544/117; 514/252.16

(57) ABSTRACT

The compounds of formula (I) are antagonists of the CCR2 receptor Wherein R¹⁻⁷ and A are as defined in the claims.

### FIELD OF THE INVENTION

The present invention relates to new pyrazolo[1,5-a]pyrimidin-7-ol compounds of general formula (I) that are antagonists of the CCR2 receptor. As such, they decrease activation of the MCP-1/CCR2 pathway involved in nociception, inflammatory processes, cancer and cancer metastasis. Accordingly, the invention also relates to pharmaceutical compositions comprising these compounds and to the use of these compounds in the treatment or prevention of medical conditions wherein mediation of the MCP-1/CCR2 pathway is beneficial, such as pain and inflammatory diseases. The invention also relates to the use of these compounds for the inhibition of the spread of metastatic tumour cells from the site of a primary tumour.

### BACKGROUND ART

Chemokines, also known as chemotactic cytokines, are a group of small proteins of low molecular-weight that are released by a wide variety of cells and have a variety of biological activities. Chemokines attract various types of cells of the immune system, such as macrophages, T cells, eosinophils, basophils and neutrophils, and cause them to migrate from the blood to various lymphoid and none-lymphoid tissues. In tumours many chemokines have been implicated in the attraction and maintenance of cancer stem cells, tumour associated macrophages, myeloid cells and other cells which are involved in tumour growth and spread. They also mediate infiltration of inflammatory cells to sites of inflammation, and are responsible for the initiation and perpetuation of many inflammation diseases (reviewed in Schall, Cytokine, 3:165-183 (1991); Schall et al., Curr. Opin. Immunol., 6:865-873 (1994)). In addition to stimulating chemotaxis, chemokines can induce other changes in responsive cells, including changes in cell shape, granule exocytosis, integrin up-regulation, formation of bioactive lipids (e.g., leukotrienes), respiratory burst associated with leukocyte activation, cell proliferation, resistance to induction of apoptosis and angiogenesis. Thus, chemokines are early triggers of the inflammatory response, causing inflammatory mediator release, chemotaxis and extravasation to sites of infection or inflammation.

The chemokine family is divided into four subfamilies, based on the number of amino acid residues between the first and second highly-conserved cysteine residues. CCR2 is one of the ten CC chemokine receptors and is found on the surface of monocytes, macrophages, B cells, activated T cells, dendritic cells, endothelial cells and tumor cells. It is a receptor for a number of chemokine ligands, including MCP-1, MCP-2, MCP-3 and MCP-4. Among them, MCP-1 (monocyte chemotactic protein-1) appears to interact only with CCR2, and not any other chemokine receptors identified so far. MCP-1 is a potent chemotactic factor and is expressed by cardiac muscle cells, blood vessel endothelial cells, fibroblasts, chondrocytes, smooth muscle cells, mesangial cells, alveolar cells, T-lymphocytes, macrophages, and the like. Following activation by its cognate ligand MCP-1, the CCR2 receptor signaling cascade involves activation of phospholipases, protein kinases, and lipid kinases.

CCR2-mediated monocyte recruitment is one of the earliest steps that leads to the development of atherosclerosis. CCR2 is expressed by monocytes and is essential to migration of these cells to the artery well, where its ligand MCP-1 is highly expressed. In experimental models of atherosclerosis, arterial plaque formation depends on the integrity of CCR2 and MCP-1, since deletion of either genes results in decreased atherosclerotic lesion formation in mice that otherwise develop severe disease (Gu et al., Mol. Cell. 2:275-281 (1998); Boring et al., Nature 394:894-897 (1998); Boring et al., J. Clin. Invest. 100:2552-2561 (1997)). The infiltration of monocytes in the inflammatory tissue and their differentiation into macrophages also provides a secondary source of several proinflammatory modulators, including tumor necrosis factor-α (TNF-α), interleukin-1 (IL-1), IL-8 (a member of the CXC chemokine subfamily), IL-12, arachidonic acid metabolites (e.g., PGE2 and LTB4), oxygen-derived free radicals, matrix metalloproteinases, and complement components.

Animal model studies of chronic inflammatory diseases have demonstrated that inhibition of binding between MCP-1 and CCR2 by an antagonist suppresses the inflammatory response. Monocyte migration is inhibited by MCP-1 antagonists (either antibodies or soluble, inactive fragments of MCP-1), which have been shown to inhibit the development of arthritis, asthma, and uveitis. Both MCP-1 and CCR2 knockout (KO) mice have demonstrated that monocyte infiltration into inflammatory lesions is significantly decreased. CCR2-mediated migration of monocytes is believed to be responsible for the pathogenicity in human multiple sclerosis (MS), as CCR2 and MCP-1 expression is observed in the cerebrospinal fluid in MS patients. In a mouse model of human MS, namely the experimental autoimmune encephalomyelitis (EAE), deficiency in CCR2 or MCP-1 prevents the development of EAE (Izikson et al., Clin. Immunol. 103:125-131 (2002); Huang et al., J. Exp. Med. 193:713-726 (2001); Fife et al., J. Exp. Med. 192:899-905 (2000); Karpus et al., J. Leukoc. Biol. 62:681-687 (1997)). The improvement seen in rheumatoid arthritis and Crohn's disease during treatment with TNF-α antagonists (e.g., monoclonal antibodies and soluble receptors) was also correlated with decreases in MCP-1 expression and the number of infiltrating macrophages. Additionally, CCR2 has recently been suggested to influence the development of obesity and associated adipose tissue inflammation and systemic insulin resistance and to play a role in the maintenance of adipose tissue macrophages and insulin resistance once obesity and its metabolic consequences are established (Weisberg et al., J. Clin. Invest., 116:115-124 (2006)). In addition, CCR2 signaling may play a pathogenic role in neuropathic pain. It has been shown that the absence of CCR2 reduces inflammatory and neuropathic pain in mouse pain models, suggesting that recruitment and activation of macrophage and microglia to neural tissues play an important role in the pain states (Abbadie et al., Proc. Natl. Acad. Sci. USA. 100:7947-7952 (2003)).

The interaction between MCP-1 and CCR2 has been linked to inflammatory disease pathologies such as psoriasis, uveitis, atherosclerosis, rheumatoid arthritis, multiple sclerosis, Crohn's disease, inflammatory bowel disease, nephritis, organ allograft rejection, fibroid lung, renal insufficiency, renal fibrosis, diabetes and diabetic complications, diabetic nephropathy, diabetic retinopathy, diabetic retinitis, diabetic microangiopathy, obesity, diabetic and other forms of neuropathy, neuropathic pain (including that associated with diabetes), tuberculosis, sarcoidosis, invasive staphylococcia, inflammation after cataract surgery, allergic rhinitis, allergic conjunctivitis, chronic urticaria, chronic obstructive pulmonary disease (COPD), allergic asthma, HIV associated dementia, periodontal diseases, periodontitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, rectum, lung, prostate and stomach (see e.g. Rollins, Mol. Med. Today, 2:198-204 (1996); Dawson et al., Expert Opin. Ther. Targets, 7(1):35-48, (2003)), Connor et al., Gut, 153:1287-1294; Ali-Osman Jr et al., J. Surg. Res., 144:350-351 (2008); Cid et al., Rheumatology, 45(11):1356-1363 (2006); Wada et al., Inflammation and regeneration, 23(5):567-572 (2004).

There remains a need for further CCR2 antagonists that can be used for preventing or treating a CCR2 mediated inflammatory disease or disorder. An aspect of the invention described here is based on the discovery that a decrease in activation of the MCP-1/CCR2 pathway in inflammatory conditions with certain pyrazolo[1,5-a]pyrimidin-7-ol molecules can effectively reduce symptoms in a subject. WO 98/54093 and WO 2004052286 disclose pyrazolo[1,5-a]pyrimidine derivatives as tyrosine kinase inhibitors for use in the treatment of cancer, diabetic retinopathy, atherosclerosis and inflammatory diseases. WO 93/17023 discloses pyrazolo[1,5-a]pyrimidin-7-ol derivatives as angiotensin (II) receptor antagonists for use in the treatment of cardiovascular diseases, in particular atherosclerosis and hypertension. Additional pyrazolo[1,5-a]pyrimidin-7-ol derivatives are disclosed as flavivirus replication inhibitors in WO 2007/005541 and as androgen inhibitors in WO 92/06096.

### CCR2, Cancer and Metastases

CCR2 and MCP-1 (as well as other CCR2 binding chemokines such as CCL7, CCL8, and CCL13 ligands (Yoshie et al. 2001) are strongly implicated in the growth, establishment and metastatic spread of a number of cancers. In general it is considered that CCR2 mediated attraction of macrophages and immunosuppressive myeloid cells to tumours and metastatic cells is the major mechanism involved, although mobilisation of a variety of bone marrow progenitor cells may also play a role. Of particular interest are the many observations that MCP-1 levels correlate with aggressiveness, invasion, macrophage content and angiogenesis in many tumour types. Plasma CCL2 levels tend to be elevated in cancer patients and are associated with tumour stage in patients with breast (Dwyer et al., 2007), ovarian (Hefler et al., 1999), and lung cancers (Cai et al., 2009).

Polymorphisms of the CCR2 receptor and MCP-1/CCL2 are significantly associated with cancer incidence in humans, including prostate, bladder, breast and cervical cancers (Zafiropoulos et al., 2004; Coelho et al., 2005; Narter et al., 2010) and cervical cancer (Chatterjee et al., 2010; Sun et al., 2011).

Cancers in which MCP-1 and CCR2 have been implicated include melanoma (Graves et al., 1992; Koga et al 2008; Zheng et al., 1999,) ovarian cancer (Negus et al., 1995), breast cancer (Saji et al., 2001; Soria et al., 2008; Soria and Ben Baruch 2008; Mestdagt et al 2004; Chavey et al., 2007; Valkovic et al., 1998; Ueno et al., 2000; Valkovic et al., 2005; Salcedo et al., 2000) oesephogeal cancer (Ohta et al., 2002; Koide et al., 2004), gastric cancer (Ohta et al., 2003; Kuroda et al., 2005; Futagami et al., 2008), renal cell carcinoma (Lukesova et al., 2008), lung cancer (Cai et al., 2009; Wong et al., 2008; Niiya et al., 2003), colon cancer (Bailey et al., 2007), thyroid cancer (Tanaka et al., 2009), leukaemia (Mazur et al., 2007), multiple myeloma (Arendt et al., 2002; Johrer et al., 2004; Van de Broeke et al., 2003; Pellegrino et al., 2005) and prostate cancer (Lu et al., 2007a).

### Prostate Cancer

Approximately 200,000 prostate cancers were diagnosed in the US in 2009, with approximately 30,000 fatalities (Jemal et al., 2009). Prostate cancer is the second major cause of cancer induced mortality in the US; partly because once metastases have reached the bone the disease is incurable.

MCP-1 promotes prostate cancer cell growth, survival, invasion and migration, as well as regulating monocytic lineage cells (i.e. macrophages and osteoclasts) which are strongly implicated in prostate cancer growth and metastasis. CCR2 mRNA and protein expression is greater within prostate cancer metastatic tissues compared to localized prostate cancer and benign prostate tissue, and correlated with a higher Gleason score suggesting that this receptor is associated with prostate cancer progression (Lu et al., 2007a).

MCP-1 induces PC-3 and VCaP cancer cell proliferation via activation of the PI3K/AKT pathway in a paracrine and autocrine manner (Loberg et al., 2006; Lu et al., 2006). The growth of subcutaneous VCaP cells can be inhibited by an anti-MCP-1 antibody which also reduced macrophage infiltration and vascularity (Loberg et al. 2007a). In metastatic colonisation experiments inhibition of MCP-1 severely inhibited overall tumour cell survival and even caused regression (Loberg et al., 2007b) as well as inhibiting colonisation of the bone (Li et al., 2009, Lu et al., 2009).

### Metastases

The establishment of metastases in bone tissue requires osteoclast mediated bone resorption (Pienta and Loberg, 2005; Taichman et al., 2007). MCP-1 promotes pre-osteoclast cell fusion with resultant formation of osteoclasts (Lu et al., 2007b), and is also involved in promoting the differentiation of CD11b+ cells into osteoclasts (Mizutani et al., 2009). Several cancers metastasize predominantly to the bone, including lung, breast kidney, thyroid cancers and multiple myeloma (see Craig and Loberg, 2006). More than 90% of patients with advanced prostate cancer presented with evidence of bone metastasis (Shah et al., 2004).

MCP-1 plays a central role in the development of bone targeted metastasis. Lu and Kang (2009) showed, using a human breast tumour line, that increased expression of MCP-1 promoted lung and bone metastasis and subsequent growth of secondary tumours. Accordingly, for the above reasons, it is expected that CCR2 blockade will be effective in inhibiting the growth of bone metastases, as well as their seeding in the lung.

The liver is the primary site of colorectal metastases, colorectal cancer being a major cause of cancer related mortality. However liver resection is seldom curative, recurrence occurring in 60-70% of cases. MCP-1 can be highly expressed in liver metastases, and high levels are associated with a poor prognosis, MCP-1 expression apparently increasing with cancer stage (i.e. being associated with increased metastatic potency, Bailey et al., 2007; Yoshimode et al., 2009).

Both liver tumour associated fibroblasts and normal fibroblasts express MCP-1 under the influence of TNFα (Muller et al., 2007, 2010), suggesting that the tumour associated fibroblasts are derived from the normal liver stroma under inflammatory conditions.

There is therefore abundant evidence for the involvement of MCP-1 and CCR2 in the growth and development of cancers, and particularly in the recruitment of cancer associated cells such as the macrophages. For the above reasons it is expected that CCR2 inhibitors will be useful in the treatment of cancers, particularly in restricting metastatic spread (from many types of cancer), and in reducing the recruitment of macrophages and myeloid cells to primary tumours, thus reducing tumour growth and vascularisation. In particular it is expected that CCR2 inhibitors will be useful for the inhibition of the spread of metastatic tumour cells from the site of a primary tumour

### DISCLOSURE OF THE INVENTION

It has surprisingly been found that the new pyrazolo[1,5-a]pyrimidin-7-ol compounds of general formula (I) are antagonists of the CCR2 receptor and can decrease activation of the MCP-1/CCR2 pathway, which is involved in nociception and inflammatory processes. The compounds are therefore potentially useful in the treatment or prevention of pain and inflammatory diseases, and for the inhibition of the spread of metastatic tumour cells from the site of a primary tumour. Consequently, the invention relates to a compound of formula (I),

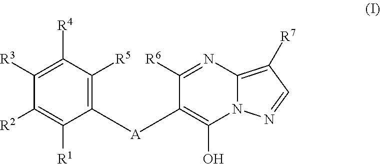

or a pharmaceutically acceptable salt, solvate, hydrate, geometrical isomer, tautomer, optical isomer or N-oxide thereof, wherein:
R¹-R⁵ are each independently selected from hydrogen, halogen, cyano, C₁₋₄-alkyl, C₁₋₄-alkoxy, fluoro-C₁₋₄-alkyl and fluoro-C₁₋₄-alkoxy;
R⁶ is selected from C₁₋₆-alkyl, fluoro-C₁₋₆-alkyl, hydroxy-C₁₋₆-alkyl, C₁₋₄-alkoxy-C₁₋₄-alkyl, C₃₋₅-cycloalkyl, C₁₋₆-alkylcarbonyl, C₁₋₆-alkoxycarbonyl, —CO₂H, heterocyclyl, heterocyclyl-C₁₋₄-alkyl, heteroaryl and heteroaryl-C₁₋₄-alkyl, wherein any heteroaryl residue is optionally substituted with C₁₋₄-alkyl;
R⁷ is selected from hydrogen, halogen, cyano, C₁₋₄-alkyl and —C(O)N(R^{8A})(R^{8B});
A is selected from —CH(R⁹)—, —N(R¹⁰)—, —O— and —S—;
R^{8A} and R^{8B} are each independently selected from hydrogen, C₁₋₄-alkyl, C₂₋₄-alkenyl, cyano-C₁₋₄-alkyl, C₁₋₄-alkoxy-C₁₋₄-alkyl, C₁₋₄-alkylthio-C₁₋₄-alkyl, —C₁₋₄-alkylene-N(R^{11A})(R^{11B}), phenyl-C₁₋₄-alkyl, phenoxy-C₁₋₄-alkyl, heteroaryl-C₁₋₄-alkyl and heterocyclyl-C₁₋₄-alkyl;
or
R^{8A} and R^{8B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl;
R⁹ and R¹⁰ are each selected from hydrogen and C₁₋₄-alkyl;
R^{11A} and R^{11B} are each independently selected from hydrogen, C₁₋₄-alkyl and phenyl;
or
R^{11A} and R^{11B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl;
provided that at least one of R¹-R⁵ is selected from halogen, cyano, C₁₋₄-alkyl, C₁₋₄-alkoxy, fluoro-C₁₋₄-alkyl or fluoro-C₁₋₄-alkoxy; and
provided that the compound is not selected from the group consisting of:
6-[(2-Chloro-4-fluorophenyl)methyl]-7-hydroxy-5-methyl-N-(3-pyridinylmethyl)-pyrazolo[1,5-a]pyrimidine-3-carboxamide;
N-(2-Cyanoethyl)-6-[(4-fluorophenyl)methyl]-7-hydroxy-N,5-dimethyl-pyrazolo[1,5-a]pyrimidine-3-carboxamide;
6-[(2-Chloro-4-fluorophenyl)methyl]-7-hydroxy-5-methyl-N-(2-phenylethyl)-pyrazolo[1,5-a]pyrimidine-3-carboxamide;
7-Hydroxy-5-methyl-6-(phenylmethyl)-pyrazolo[1,5-a]pyrimidine-3-carbonitrile;
N-[2-(Butylmethylamino)ethyl]-6-[(2-fluorophenyl)methyl]-7-hydroxy-5-methyl-pyrazolo[1,5-a]pyrimidine-3-carboxamide;
6-[(4-Chlorophenyl)methyl]-7-hydroxy-N,5-dimethyl-N-(phenylmethyl)-pyrazolo[1,5-a]pyrimidine-3-carboxamide;
6-[(3-Chlorophenyl)methyl]-7-hydroxy-5-methyl-N-(2-phenylethyl)-pyrazolo[1,5-a]pyrimidine-3-carboxamide;
N-Butyl-6-[(4-fluorophenyl)methyl]-7-hydroxy-N,5-dimethyl-pyrazolo[1,5-a]pyrimidine-3-carboxamide;
N-Butyl-6-[(2-chlorophenyl)methyl]-7-hydroxy-5-methyl-pyrazolo[1,5-a]pyrimidine-3-carboxamide;
[6-[(4-Chlorophenyl)methyl]-7-hydroxy-5-methylpyrazolo[1,5-a]pyrimidin-3-yl]-1-pyrrolidinyl-methanone;
[6-[(3-Methylphenyl)methyl]-7-hydroxy-5-methylpyrazolo[1,5-a]pyrimidin-3-yl][4-ethyl-1-piperazinyl]-methanone;
6-[(2-Chloro-4-fluorophenyl)methyl]-7-hydroxy-5-methyl-N-[3-(4-morpholinyl)propyl]-pyrazolo[1,5-a]pyrimidine-3-carboxamide;
6-[(4-Chlorophenyl)methyl]-7-hydroxy-N-(2-methoxyethyl)-5-methyl-pyrazolo[1,5-a]pyrimidine-3-carboxamide;
[6-[(2-Fluorophenyl)methyl]-7-hydroxy-5-methylpyrazolo[1,5-a]pyrimidin-3-yl]-1-piperidinyl-methanone;
N-[3-(2-Ethyl-1-piperidinyl)propyl]-6-[(2-fluorophenyl)methyl]-7-hydroxy-5-methyl-pyrazolo[1,5-a]pyrimidine-3-carboxamide;
6-[(2-Fluorophenyl)methyl]-7-hydroxy-5-methyl-N-(2-phenylethyl)-pyrazolo[1,5-a]pyrimidine-3-carboxamide,
[6-[(4-Chorophenyl)methyl]-7-hydroxy-5-methylpyrazolo[1,5-a]pyrimidin-3-yl]-1-piperidinyl-methanone;
6-[(2-Fluorophenyl)methyl]-7-hydroxy-N,5-dimethyl-N-(phenylmethyl)-pyrazolo[1,5-a]pyrimidine-3-carboxamide;
N-[2-(Dimethylamino)ethyl]-6-[(2-fluorophenyl)methyl]-7-hydroxy-5-methyl-pyrazolo[1,5-a]pyrimidine-3-carboxamide;
[6-[(2-Chlorophenyl)methyl]-7-hydroxy-5-methylpyrazolo[1,5-a]pyrimidin-3-yl]-1-pyrrolidinyl-methanone;
[6-[(2-Chloro-4-fluorophenyl)methyl]-7-hydroxy-5-methylpyrazolo[1,5-a]pyrimidin-3-yl]-1-pyrrolidinyl-methanone;
[6-[(4-Chorophenyl)methyl]-7-hydroxy-5-methylpyrazolo[1,5-a]pyrimidin-3-yl]-4-morpholinyl-methanone;
[6-[(4-Methylphenyl)methyl]-7-hydroxy-5-methylpyrazolo[1,5-a]pyrimidin-3-yl]-1-pyrrolidinyl-methanone;
6-[(4-Fluorophenyl)methyl]-7-hydroxy-5-methyl-N-[2-(4-morpholinyl)ethyl]-pyrazolo[1,5-a]pyrimidine-3-carboxamide;
[6-[(2-Chloro-4-fluorophenyl)methyl]-7-hydroxy-5-methylpyrazolo[1,5-a]pyrimidin-3-yl]-4-morpholinyl-methanone;
6-[(2-Chlorophenyl)methyl]-7-hydroxy-5-methyl-N-(phenylmethyl)-pyrazolo[1,5-a]pyrimidine-3-carboxamide;
6-[(2-Fluorophenyl)methyl]-N-(2-furanylmethyl)-7-hydroxy-N,5-dimethyl-pyrazolo[1,5-a]pyrimidine-3-carboxamide;
6-[(4-Chlorophenyl)methyl]-N-[3-(diethylamino)propyl]-7-hydroxy-5-methyl-pyrazolo[1,5-a]pyrimidine-3-carboxamide;
N-[2-(Ethylphenylamino)ethyl]-6-[(2-fluorophenyl)methyl]-7-hydroxy-5-methyl-pyrazolo[1,5-a]pyrimidine-3-carboxamide;
6-[(2-Chlorophenyl)methyl]-7-hydroxy-5-methyl-N-(1-methylpropyl)-pyrazolo[1,5-a]pyrimidine-3-carboxamide; and
6-[(2-Fluorophenyl)methyl]-7-hydroxy-N,5-dimethyl-pyrazolo[1,5-a]pyrimidine-3-carboxamide.

Another object of the present invention is a compound of formula (I) as defined above for use in therapy, provided that the compound is not selected from the group consisting of:
N-[2-(Ethylphenylamino)ethyl]-6-[(2-fluorophenyl)methyl]-7-hydroxy-5-methyl-pyrazolo[1,5-a]pyrimidine-3-carboxamide;
6-[(2-Chlorophenyl)methyl]-7-hydroxy-5-methyl-N-(1-methylpropyl)-pyrazolo[1,5-a]-pyrimidine-3-carboxamide; and
6-[(2-Fluorophenyl)methyl]-7-hydroxy-N,5-dimethyl-pyrazolo[1,5-a]pyrimidine-3-carboxamide.

### R¹-R⁵

The groups R1, R2, R3, R4, and R5, R¹-R⁵ are each independently selected from hydrogen; halogen for example fluoro, chloro, bromo; cyano; C₁₋₄-alkyl for example methyl, ethyl, n-propyl, isopropyl, n-butyl, isobutyl, sec-butyl, t-butyl; C₁₋₄-alkoxy for example methoxy, ethoxy, n-propoxy, isopropoxy, n-butoxy, isobutoxy, sec-butoxy and tert-butoxy; fluoro-C₁₋₄-alkyl for example fluoromethyl, trifluoromethyl, 2-fluoroethyl and 2,2,2-trifluoroethyl; and fluoro-C₁₋₄-alkoxy for example trifluoromethoxy and 2,2,2-trifluoroethoxy.

In a preferred embodiment R¹-R⁵ are independently selected from hydrogen, halogen, methyl, methoxy, CF₃ and OCF₃. In a yet more preferred embodiment R¹-R⁵ are independently selected from hydrogen, fluoro, chloro, bromo and CF₃.

In alternative preferred embodiments R¹ is hydrogen, and R²-R⁵ are independently selected from fluoro, chloro, bromo and CF₃; or R¹ and R⁵ are hydrogen, and R²-R⁴ are each independently selected from fluoro, chloro, bromo and CF₃; or R¹, R², and R⁵ are hydrogen, and R² and R³ are each independently selected from fluoro, chloro, bromo and CF₃; or R¹, R³, and R⁵ are hydrogen, and R² and R⁴ are each independently selected from fluoro, chloro, bromo and CF₃; or R¹, R², R⁴, and R⁵ are hydrogen, and R³ is selected from fluoro, chloro, bromo and CF₃; or R¹, R³, R⁴, and R⁵ are hydrogen, and R² is selected from fluoro, chloro, bromo and CF₃. It is particularly preferred that R² and R³ are independently selected from fluoro and CF₃; or R² and R⁴ are independently selected from fluoro and CF₃.

### R⁶

The group R⁶ is selected from C₁₋₆-alkyl for example methyl, ethyl, n-propyl, isopropyl, n-butyl, isobutyl, sec-butyl, t-butyl and straight- and branched-chain pentyl and hexyl; fluoro-C₁₋₆-alkyl for example fluoromethyl, trifluoromethyl, 2-fluoroethyl and 2,2,2-trifluoroethyl; hydroxy-C₁₋₆-alkyl for example hydroxymethyl, 2-hydroxyethyl, 2-hydroxypropyl and 2-hydroxy-2-methylpropyl; C₁₋₄-alkoxy-C₁₋₄-alkyl for example methoxymethyl, methoxyethyl, ethoxyethyl, isopropoxyethyl, n-butoxyethyl and t-butoxyethyl; C₃₋₅-cycloalkyl for example cyclopropyl, cyclobutyl, cyclopentyl; C₁₋₆-alkylcarbonyl for example methylcarbonyl (acetyl), ethylcarbonyl and n-propylcarbonyl; C₁₋₆-alkoxycarbonyl for example methoxycarbonyl, ethoxycarbonyl and isopropoxycarbonyl; —CO₂H; heterocyclyl for example piperidinyl, tetrahydropyranyl, tetrahydrofuranyl, oxetanyl, azetidinyl, pyrrolidinyl, morpholinyl, imidazolidinyl, thiomorpholinyl, dioxanyl, piperazinyl and homopiperazinyl; heterocyclyl-C₁₋₄-alkyl for example piperidin-1-ylmethyl, piperidin-4-ylmethyl and morpholin-4-ylmethyl; heteroaryl for example furyl, pyrrolyl, thienyl, oxazolyl, isoxazolyl, imidazolyl, thiazolyl, isothiazolyl, pyridinyl, pyrimidinyl, tetrazolyl, quinazolinyl, indolyl, indolinyl, isoindolyl, isoindolinyl, pyrazolyl, pyridazinyl, pyrazinyl, quinolinyl, quinoxalinyl, oxadiazolyl, thiadiazolyl, benzofuranyl, 2,3-dihydrobenzofuranyl, 1,3-benzodioxolyl, 1,4-benzodioxinyl, benzothiazolyl, benzimidazolyl, benzotriazolyl and chromanyl; and heteroaryl-C₁₋₄-alkyl such as 2-(pyridin-2-yl)-ethyl and 1,2,4-oxadiazol-5-ylmethyl, wherein any heteroaryl residue is optionally substituted with C₁₋₄-alkyl.

In another preferred embodiment, R⁶ is selected from C₁₋₄-alkyl, fluoro-C₁₋₄-alkyl, hydroxy-C₁₋₄-alkyl, C₁₋₄-alkoxy-C₁₋₄-alkyl, C₃₋₅-cycloalkyl, and C₁₋₄-alkoxycarbonyl. More preferably, R⁶ is selected from C₁₋₃-alkyl for example methyl, ethyl, propyl, isopropyl; C₃₋₄-cycloalkyl for example cyclopropyl or cyclobutyl; and C₁₋₃-alkoxycarbonyl for example methoxycarbonyl, ethoxycarbonyl and isopropoxycarbonyl. Yet more preferably, R⁶ is selected from ethyl, isopropyl, cyclopropyl, or cyclobutyl. In a particularly preferred embodiment, R⁶ is selected from isopropyl or cyclopropyl.

### R⁷

The group R⁷ is selected from hydrogen; halogen for example fluoro, chloro, bromo; cyano; C₁₋₄-alkyl for example methyl, ethyl, n-propyl, isopropyl, and —C(O)N(R^{8A})(R^{8B}). In a preferred embodiment R⁷ is hydrogen.

### A

The group A is selected from —CH(R⁹)—, —N(R¹⁰)—, —O— and —S—, wherein R⁹ and R¹⁰ are as defined above, for example hydrogen, or methyl, ethyl, n-propyl, isopropyl. In a preferred embodiment A is selected from —CH(R⁹)—, where R⁹ is as defined and discussed above, and —O—. When A is —CH(R⁹)— a currently preferred embodiment is where R⁹ is hydrogen.

### R^{8A} and R^{8B}

The groups R^{8A} and R^{8B} are each independently selected from hydrogen, C₁₋₄-alkyl for example methyl, ethyl, n-propyl, isopropyl; C₂₋₄-alkenyl for example allyl; cyano-C₁₋₄-alkyl for example cyanoethyl, C₁₋₄-alkoxy-C₁₋₄-alkyl for example methoxymethyl, methoxyethyl, methoxypropyl, ethoxyethyl, isopropoxyethyl, n-butoxyethyl and t-butoxyethyl; C₁₋₄-alkylthio-C₁₋₄-alkyl for example 2-(methylsulfanyl)ethyl and 2-(ethylsulfanyl)ethyl; —C₁₋₄-alkylene-N(R^{11A})(R^{11B}), phenyl-C₁₋₄-alkyl for example phenethyl; phenoxy-C₁₋₄-alkyl for example phenoxyethyl; heteroaryl-C₁₋₄-alkyl for example 2-pyridylethyl; and heterocyclyl-C₁₋₄-alkyl for example 2-methyl furan;

or
The groups R^{8A} and R^{8B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl, examples of such ring systems include for example morpholine, and 4-methyl piperazine.

Specific preferred compounds of formula (I) are the compounds selected from the group consisting of:
6-[(4-Chlorophenyl)methyl]-5-methylpyrazolo[1,5-a]pyrimidin-7-ol;
5-Methyl-6-{[4-(trifluoromethyl)phenyl]methyl}pyrazolo[1,5-a]pyrimidin-7-ol;
3-[(3-Fluorophenyl)methyl]-2-(pyrazin-2-yl)imidazo[1,5-a]pyrimidin-4-ol;
5-Ethyl-6-{[3-fluoro-5-(trifluoromethyl)phenyl]methyl}pyrazolo[1,5-a]pyrimidin-7-ol;
6-(3-Fluorophenoxy)-5-methylpyrazolo[1,5-a]pyrimidin-7-ol;
Methyl 6-[(4-chlorophenyl)methyl]-7-hydroxypyrazolo[1,5-a]pyrimidine-5-carboxylate;
6-[(2,3-Difluorophenyl)methyl]-5-methylpyrazolo[1,5-a]pyrimidin-7-ol;
2-Fluoro-5-({7-hydroxy-5-methylpyrazolo[1,5-a]pyrimidin-6-yl}methyl)benzonitrile;
6-[(3-Bromo-4-chlorophenyl)methyl]-5-methylpyrazolo[1,5-a]pyrimidin-7-ol;
6-[(3-Methoxyphenyl)methyl]-5-methylpyrazolo[1,5-a]pyrimidin-7-ol;
6-[1-(4-Chlorophenyl)ethyl]-5-methylpyrazolo[1,5-a]pyrimidin-7-ol;
6-[(3,5-difluorophenyl)methyl]-5-ethylpyrazolo[1,5-a]pyrimidin-7-ol;
5-Ethyl-6-[(3,4,5-trifluorophenyl)methyl]pyrazolo[1,5-a]pyrimidin-7-ol;
6-[(3-Chloro-4-fluorophenyl)methyl]-5-ethylpyrazolo[1,5-a]pyrimidin-7-ol;
6-{[3-Fluoro-4-(trifluoromethyl)phenyl]methyl}-5-propylpyrazolo[1,5-a]pyrimidin-7-ol;
6-[(3,4-Dichlorophenyl)methyl]-5-propylpyrazolo[1,5-a]pyrimidin-7-ol;
6-Benzyl-5-cyclopropylpyrazolo[1,5-a]pyrimidin-7-ol;
5-Cyclopropyl-6-{[4-(trifluoromethoxy)phenyl]methyl}pyrazolo[1,5-a]pyrimidin-7-ol;
5-Cyclopropyl-6-{[3-(trifluoromethoxy)phenyl]methyl}pyrazolo[1,5-a]pyrimidin-7-ol;
6-[(3-Chlorophenyl)methyl]-5-cyclopropylpyrazolo[1,5-a]pyrimidin-7-ol;
5-Cyclopropyl-6-{[3-(trifluoromethyl)phenyl]methyl}pyrazolo[1,5-a]pyrimidin-7-ol;
5-Cyclopropyl-6-{[4-(trifluoromethyl)phenyl]methyl}pyrazolo[1,5-a]pyrimidin-7-ol;
5-Cyclopropyl-6-[(3,4-difluorophenyl)methyl]pyrazolo[1,5-a]pyrimidin-7-ol;
5-Cyclopropyl-6-[(3,5-difluorophenyl)methyl]pyrazolo[1,5-a]pyrimidin-7-ol;
5-Cyclopropyl-6-[(3,4,5-trifluorophenyl)methyl]pyrazolo[1,5-a]pyrimidin-7-ol;
6-[4-Chloro-3-fluorophenyl)methyl]-5-cyclopropylpyrazolo[1,5-a]pyrimidin-7-ol;
6-[(3-Chloro-4-fluorophenyl)methyl]-5-cyclopropylpyrazolo[1,5-a]pyrimidin-7-ol;
6-[(3-Chloro-5-fluorophenyl)methyl]-5-cyclopropylpyrazolo[1,5-a]pyrimidin-7-ol;
5-Cyclopropyl-6-[(3,4-dichlorophenyl)methyl]pyrazolo[1,5-a]pyrimidin-7-ol;
5-Cyclopropyl-6-{[4-fluoro-3-(trifluoromethyl)phenyl]methyl}pyrazolo[1,5-a]pyrimidin-7-ol;
5-Cyclopropyl-6-{[3-fluoro-5-(trifluoromethyl)phenyl]methyl}pyrazolo[1,5-a]pyrimidin-7-ol;
5-Cyclopropyl-6-[(3,5-dichlorophenyl)methyl]pyrazolo[1,5-a]pyrimidin-7-ol;
6-[(3,4-Difluorophenyl)methyl]-5-(propan-2-yl)pyrazolo[1,5-a]pyrimidin-7-ol;
5-(Propan-2-yl)-6-[(3,4,5-trifluorophenyl)methyl]pyrazolo[1,5-a]pyrimidin-7-ol;
6-[(3-Chloro-4-fluorophenyl)methyl]-5-(propan-2-yl)pyrazolo[1,5-a]pyrimidin-7-ol;
6-[(4-Chloro-3-fluorophenyl)methyl]-5-(propan-2-yl)pyrazolo[1,5-a]pyrimidin-7-ol;
6-[(3-Chloro-5-fluorophenyl)methyl]-5-(propan-2-yl)pyrazolo[1,5-a]pyrimidin-7-ol;
6-{[4-Fluoro-3-(trifluoromethyl)phenyl]methyl}-5-(propan-2-yl)pyrazolo[1,5-a]pyrimidin-7-ol;
6-[(3,4-Dichlorophenyl)methyl]-5-(propan-2-yl)pyrazolo[1,5-a]pyrimidin-7-ol;
6-[(3,5-Dichlorophenyl)methyl]-5-(propan-2-yl)pyrazolo[1,5-a]pyrimidin-7-ol;
6-[(3-Chlorophenyl)methyl]-5-cyclobutylpyrazolo[1,5-a]pyrimidin-7-ol;
6-[(4-Chlorophenyl)methyl]-5-cyclobutylpyrazolo[1,5-a]pyrimidin-7-ol;
5-Cyclobutyl-6-[(3,4-difluorophenyl)methyl]pyrazolo[1,5-a]pyrimidin-7-ol;
6-[(3-Chloro-4-fluorophenyl)methyl]-5-cyclobutylpyrazolo[1,5-a]pyrimidin-7-ol;
5-Cyclobutyl-6-{[4-fluoro-3-(trifluoromethyl)phenyl]methyl}pyrazolo[1,5-a]pyrimidin-7-ol;
5-Cyclobutyl-6-{[3-fluoro-5-(trifluoromethyl)phenyl]methyl}pyrazolo[1,5-a]pyrimidin-7-ol;
5-Cyclobutyl-6-[(3,4,5-trifluorophenyl)methyl]pyrazolo[1,5-a]pyrimidin-7-ol;
5-Cyclobutyl-6-[(3,4-dichlorophenyl)methyl]pyrazolo[1,5-a]pyrimidin-7-ol;
6-[(4-Chlorophenyl)methyl]-5-(methoxymethyl)pyrazolo[1,5-a]pyrimidin-7-ol;
6-[(4-Chloro-3-fluorophenyl)methyl]-5-(methoxymethyl)pyrazolo[1,5-a]pyrimidin-7-ol;
6-[(3,4-Dichorophenyl)methyl]-5-(methoxymethyl)pyrazolo[1,5-a]pyrimidin-7-ol;
6-[(4-Chlorophenyl)methyl]-5-(2-methoxyethyl)pyrazolo[1,5-a]pyrimidin-7-ol;
6-[(3,4-Dichorophenyl)methyl]-5-(trifluoromethyl)pyrazolo[1,5-a]pyrimidin-7-ol;
6-[(3,4-Dichlorophenyl)methyl]-5-(oxolan-2-yl)pyrazolo[1,5-a]pyrimidin-7-ol;
6-[(4-Chlorophenyl)methyl]-3,5-dimethylpyrazolo[1,5-a]pyrimidin-7-ol;
3-Bromo-6-[(4-chlorophenyl)methyl]-5-methylpyrazolo[1,5-a]pyrimidin-7-ol;
6-[(4-Chlorophenyl)methyl]-7-hydroxy-5-methylpyrazolo[1,5-a]pyrimidine-3-carbonitrile;
6-(3,4-Dichlorophenoxy)-5-methylpyrazolo[1,5-a]pyrimidin-7-ol;
5-Ethyl-6-[3-(trifluoromethyl)phenoxy]pyrazolo[1,5-a]pyrimidin-7-ol;
6-(3,4-Difluorophenoxy)-5-ethylpyrazolo[1,5-a]pyrimidin-7-ol
6-(3-Chloro-4-fluorophenoxy)-5-ethylpyrazolo[1,5-a]pyrimidin-7-ol;
6-(3,4-Dichlorophenoxy)-5-ethylpyrazolo[1,5-a]pyrimidin-7-ol;
6-(4-Bromophenoxy)-5-cyclopropylpyrazolo[1,5-a]pyrimidin-7-ol;
6-(4-Bromophenoxy)-5-(propan-2-yl)pyrazolo[1,5-a]pyrimidin-7-ol;
6-(4-Chloro-3-fluorophenoxy)-5-(propan-2-yl)pyrazolo[1,5-a]pyrimidin-7-ol;
6-(3,4-diChlorophenoxy)-5-(propan-2-yl)pyrazolo[1,5-a]pyrimidin-7-ol;
Methyl 6-[(3-fluorophenyl)methyl]-7-hydroxypyrazolo[1,5-a]pyrimidine-5-carboxylate;
Ethyl 6-[(3-chlorophenyl)methyl]-7-hydroxypyrazolo[1,5-a]pyrimidine-5-carboxylate;
Ethyl 7-hydroxy-6-{[3-(trifluoromethyl)phenyl]methyl}pyrazolo[1,5-a]pyrimidine-5-carboxylate;
Ethyl 6-[(3,4-dichlorophenyl)methyl]-7-hydroxypyrazolo[1,5-a]pyrimidine-5-carboxylate;
Ethyl 6-[(3-fluorophenyl)methyl]-7-hydroxypyrazolo[1,5-a]pyrimidine-5-carboxylate;
Ethyl 6-[(4-chloro-3-fluorophenyl)methyl]-7-hydroxy pyrazolo[1,5-a]pyrimidine-5-carboxylate;
Ethyl 6-(4-chloro-3-fluorophenoxy)-7-hydroxypyrazolo[1,5-a]pyrimidine-5-carboxylate;
Ethyl 6-(3,4-dichlorophenoxy)-7-hydroxypyrazolo[1,5-a]pyrimidine-5-carboxylate;
6-[(3-Fluorophenyl)sulfanyl]-5-methylpyrazolo[1,5-a]pyrimidin-7-ol,
6-[(3-Fluorophenyl)amino]-5-methylpyrazolo[1,5-a]pyrimidin-7-ol;
6-[Ethyl(3-fluorophenyl)amino]-5-methylpyrazolo[1,5-a]pyrimidin-7-ol;
6-[(3-Fluorophenyl)(methyl)amino]-5-methylpyrazolo[1,5-a]pyrimidin-7-ol;
Methyl 6-[(3-chlorophenyl)methyl]-7-hydroxypyrazolo[1,5-a]pyrimidine-5-carboxylate;
Propan-2-yl6-[(3-chlorophenyl)methyl]-7-hydroxypyrazolo[1,5-a]pyrimidine-5-carboxylate;
Methyl 6-[(3,4-dichlorophenyl)methyl]-7-hydroxypyrazolo[1,5-a]pyrimidine-5-carboxylate;
Propan-2-yl6-[(3,4-dichlorophenyl)methyl]-7-hydroxy pyrazolo[1,5-a]pyrimidine-5-carboxylate;
6-[(3-Chlorophenyl)methyl]-7-hydroxypyrazolo[1,5-a]pyrimidine-5-carboxylic acid;
Propan-2-yl7-hydroxy-6-{[3-(trifluoromethyl)phenyl]methyl}pyrazolo[1,5-a]pyrimidine-5-carboxylate;
Propan-2-yl6-[(3-chloro-4-fluoro-phenyl)methyl]-7-hydroxypyrazolo[1,5-a]pyrimidine-5-carboxylate;
Propan-2-yl6-[(4-chloro-3-Fluorophenyl)methyl]-7-hydroxypyrazolo[1,5-a]pyrimidine-5-carboxylate;
Methyl 6-(3,4-dichlorophenoxy)-7-hydroxypyrazolo[1,5-a]pyrimidine-5-carboxylate;
6-[(4-Chorophenyl)methyl]-5-(hydroxymethyl)pyrazolo[1,5-a]pyrimidin-7-ol;
6-[(4-Chorophenyl)methyl]-5-(morpholin-4-ylmethyl)pyrazolo[1,5-a]pyrimidin-7-ol;
6-[(3-Fluorophenyl)methyl]-5-(3-methyl-1,2,4-oxadiazol-5-yl)pyrazolo[1,5-a]pyrimidin-7-ol;
6-[(3-Fluorophenyl)methyl]-5-(2-hydroxypropan-2-yl)pyrazolo[1,5-a]pyrimidin-7-ol;
1-{6-[(3-Fluorophenyl)methyl]-7-hydroxypyrazolo[1,5-a]pyrimidin-5-yl}ethan-1-one;
6-[(3-Fluorophenyl)methyl]-5-(1-hydroxyethyl)pyrazolo[1,5-a]pyrimidin-7-ol;
1-{6-[(3,4-Dichlorophenyl)methyl]-7-hydroxypyrazolo[1,5-a]pyrimidin-5-yl}ethan-1-one;
1-{6-[(3,4-Dichlorophenyl)methyl]-7-hydroxypyrazolo[1,5-a]pyrimidin-5-yl}propan-1-one;
6-[(4-Chorophenyl)methyl]-5-[(3-methyl-1,2,4-oxadiazol-5-yl)methyl]pyrazolo[1,5-a]pyrimidin-7-ol;
6-[(4-Chorophenyl)methyl]-7-hydroxy-N,5-dimethylpyrazolo[1,5-a]pyrimidine-3-carboxamide;
6-[(4-Chorophenyl)methyl]-7-hydroxy-N,N,5-trimethylpyrazolo[1,5-a]pyrimidine-3-carboxamide;
6-[(4-Chlorophenyl)methyl]-7-hydroxy-5-methyl-N-[(2R)-oxolan-2-ylmethyl]pyrazolo-[1,5-a]pyrimidine-3-carboxamide;
7-Hydroxy-N-(2-methoxyethyl)-5-methyl-6-[(4-methylphenyl)methyl]pyrazolo[1,5-a]pyrimidine-3-carboxamide;
6-Benzyl-7-hydroxy-5-methyl-N-(2-phenoxyethyl)pyrazolo[1,5-a]pyrimidine-3-carboxamide;
6-[(4-Chorophenyl)methyl]-5-methyl-3-[(4-methylpiperazin-1-yl)carbonyl]pyrazolo[1,5-a]pyrimidin-7-ol;
6-[(4-Chorophenyl)methyl]-7-hydroxy-5-methyl-N-(2-phenylethyl)pyrazolo[1,5-a]pyrimidine-3-carboxamide;
6-[(4-Chorophenyl)methyl]-7-hydroxy-5-methyl-N-(prop-2-en-1-yl)pyrazolo[1,5-a]-pyrimidine-3-carboxamide;
6-[(4-Chlorophenyl)methyl]-7-hydroxy-5-methyl-N-[2-(methylsulfanyl)ethyl]pyrazolo[1,5-a]pyrimidine-3-carboxamide;
6-[(4-Chlorophenyl)methyl]-N-(2-cyanoethyl)-7-hydroxy-N,5-dimethylpyrazolo[1,5-a]pyrimidine-3-carboxamide;
6-[(4-Chlorophenyl)methyl]-7-hydroxy-5-methyl-N-(oxolan-2-ylmethyl)pyrazolo[1,5-a]pyrimidine-3-carboxamide;
7-Hydroxy-5-methyl-6-[(4-methylphenyl)methyl]-N-[2-(pyridin-2-yl)ethyl]pyrazolo[1,5-a]pyrimidine-3-carboxamide;
6-Benzyl-7-hydroxy-N-(3-methoxypropyl)-5-methylpyrazolo[1,5-a]pyrimidine-3-carboxamide;
7-Hydroxy-N-(2-methoxyethyl)-5-methyl-6-{[4-(trifluoromethyl)phenyl]methyl}pyrazolo[1,5-a]pyrimidine-3-carboxamide;
5-Methyl-3-(morpholin-4-ylcarbonyl)-6-{[4-(trifluoromethyl)phenyl]ethyl}pyrazolo[1,5-a]pyrimidin-7-ol;
6-[(3,4-Dichlorophenyl)methyl]-7-hydroxy-N-(2-methoxyethyl)-5-methylpyrazolo[1,5-a]pyrimidine-3-carboxamide;
6-[(3-Chlorophenyl)methyl]-7-hydroxy-N-(2-methoxyethyl)-5-methylpyrazolo[1,5-a]-pyrimidine-3-carboxamide;
6-[(4-Chlorophenyl)methyl]-5-ethyl-7-hydroxy-N-(2-methoxyethyl)pyrazolo[1,5-a]pyrimidine-3-carboxamide;
6-[(4-Chlorophenyl)methyl]-7-hydroxy-N-(2-methoxyethyl)-5-(methoxymethyl)-pyrazolo[1,5-a]pyrimidine-3-carboxamide;
6-[(3,4-Dichlorophenyl)methyl]-5-ethylpyrazolo[1,5-a]pyrimidin-7-ol; and
5-Cyclopropyl-6-{[3-fluoro-4-(trifluoromethyl)phenyl]methyl}pyrazolo[1,5-a]pyrimidin-7-ol.

The compounds of formula (I) are useful as antagonists of the CCR2 receptor. As such, they are useful in the treatment or prevention of medical conditions and diseases in which mediation of the MCP-1/CCR2 pathway is beneficial, such as pain and inflammatory diseases. In particular, it is believed that compounds of formula (I) are useful for the treatment or prevention of psoriasis, uveitis, atherosclerosis, rheumatoid arthritis, multiple sclerosis, inflammatory bowel disease, Crohn's disease, nephritis, lupus and lupus nephritis, organ allograft rejection, fibroid lung, renal insufficiency, IgA nephropathy, renal fibrosis, diabetes and diabetic complications, diabetic nephropathy, diabetic retinopathy, diabetic retinitis, diabetic microangiopathy, obesity, diabetic and other forms of neuropathy, neuropathic pain (including that associated with diabetes), chronic pain, giant cell arteritis and other vasculitic inflammatory diseases, tuberculosis, sarcoidosis, invasive staphylococcia, inflammation after cataract surgery, allergic rhinitis, allergic conjunctivitis, chronic urticaria, chronic obstructive pulmonary disease (COPD), allergic asthma, HIV associated dementia, periodontal diseases, periodontitis, gingivitis, gum disease, diastolic cardiomyopathies, cardiac infarction, myocarditis, chronic heart failure, angiostenosis, restenosis, reperfusion disorders, glomerulonephritis (including but not restricted to focal and segmental glomerulosclerosis, IgA glomerulonephritis, IgM glomerulonephritis, membranoproliferative glomerulonephritis, membranous glomerulonephritis, minimal change nephropathy, vasculitis (including microscopic polyarteritis, Wegener's granulomatosis, Henoch Schonlein purpura and polyarteritis nodosa,)), solid tumors and cancers, chronic lymphocytic leukemia, chronic myelocytic leukemia, multiple myeloma, malignant myeloma, Hodgkin's disease, and carcinomas of the bladder, breast, cervix, colon, rectum, lung, prostate and stomach.

It is also believed that the compounds of formula (I) are useful for the inhibition of the spread of metastatic tumour cells from the site of a primary tumour.

Another object of the invention thus is the use of compounds of formula (I) in the manufacture of a medicament for the treatment or prevention of the above-mentioned medical conditions and diseases. Yet another object of the invention is a method for treatment or prevention of such medical conditions and diseases, comprising administering to a mammal, including man, in need of such treatment an effective amount of a compound of formula (I) as defined above.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabeling/assay techniques, blotting/chemiluminescence methods, real-time PCR, and the like.

### DEFINITIONS

The following definitions shall apply throughout the specification and the appended claims, unless otherwise stated or indicated.

The term “C₁₋₆-alkyl” denotes a straight or branched alkyl group having from 1 to 6 carbon atoms. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. Examples of said “C₁₋₆-alkyl” include methyl, ethyl, n-propyl, isopropyl, n-butyl, isobutyl, sec-butyl, t-butyl and straight- and branched-chain pentyl and hexyl.

The term “fluoro-C₁₋₆-alkyl” denotes a straight or branched C₁₋₆-alkyl group substituted by one or more fluorine atoms. Examples of said fluoro-C₁₋₆-alkyl include fluoromethyl, trifluoromethyl, 2-fluoroethyl and 2,2,2-trifluoroethyl.

The term “hydroxy-C₁₋₆-alkyl” denotes a straight or branched C₁₋₆-alkyl group that has one or more hydrogen atoms thereof replaced with OH. Examples of said hydroxy-C₁₋₆-alkyl include hydroxymethyl, 2-hydroxyethyl, 2-hydroxypropyl and 2-hydroxy-2-methylpropyl.

The term “C₁₋₆-alkoxy” refers to a straight or branched C₁₋₆-alkyl group which is attached to the remainder of the molecule through an oxygen atom. For parts of the range C₁₋₆-alkoxy, all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, etc. Examples of said C₁₋₆-alkoxy include methoxy, ethoxy, n-propoxy, isopropoxy, n-butoxy, isobutoxy, sec-butoxy and tert-butoxy.

The term “fluoro-C₁₋₄-alkoxy” denotes a fluoro-C₁₋₄-alkyl group which is attached to the remainder of the molecule through an oxygen atom. Exemplary fluoro-C₁₋₄-alkoxy groups include trifluoromethoxy and 2,2,2-trifluoroethoxy.

The term “C₁₋₄-alkoxy-C₁₋₄-alkyl” denotes a straight or branched alkoxy group having from 1 to 4 carbon atoms connected to a straight or branched alkyl group having from 1 to 4 carbon atoms. Examples of said C₁₋₄-alkoxy-C₁₋₄-alkyl include methoxymethyl, methoxyethyl, ethoxyethyl, isopropoxyethyl, n-butoxyethyl and t-butoxyethyl.

The term “C₁₋₄-alkylthio-C₁₋₄-alkyl” denotes a straight or branched C₁₋₄-alkyl group that is attached through a sulfur atom to a straight or branched C₁₋₄-alkyl group. Examples of said C₁₋₄-alkylthio-C₁₋₄-alkyl include 2-(methylsulfanyl)ethyl and 2-(ethylsulfanyl)ethyl.

The term “cyano-C₁₋₄-alkyl” denotes a straight or branched C₁₋₄-alkyl group substituted by one or more cyano groups. Exemplary cyano-C₁₋₄-alkyl groups include 2-cyanoethyl and 3-cyanopropyl.

The term “C₁₋₆-alkylcarbonyl” denotes a straight or branched C₁₋₆-alkyl group that is attached to a carbonyl group. Examples of said C₁₋₆-alkylcarbonyl include methylcarbonyl (acetyl), ethylcarbonyl and n-propylcarbonyl.

The term “C₁₋₆-alkoxycarbonyl” denotes a straight or branched C₁₋₆-alkoxy group that is attached to a carbonyl group. Examples of said C₁₋₆-alkoxycarbonyl include methoxycarbonyl, ethoxycarbonyl and isopropoxycarbonyl.

The term “C₃₋₅-cycloalkyl” denotes a saturated monocyclic hydrocarbon ring having from 3 to 5 carbon atoms. Examples of said C₃₋₅-cycloalkyl include cyclopropyl, cyclobutyl and cyclopentyl.

The term “phenyl-C₁₋₄-alkyl” denotes a phenyl group that is directly linked to a straight or branched C₁₋₄-alkyl group. Examples of such groups include phenylmethyl (i.e., benzyl) and 2-phenylethyl.

The term “phenoxy-C₁₋₄-alkyl” denotes a phenyl group that is linked to a straight or branched C₁₋₄-alkyl group through an oxygen atom. Examples of such groups include phenoxymethyl and phenoxyethyl.

The term “heterocyclyl” or “heterocyclic ring” denotes a saturated, monocyclic ring having from 4 to 7 ring atoms with at least one heteroatom such as O, N, or S, and the remaining ring atoms are carbon. Examples of heterocyclic rings include piperidinyl, tetrahydropyranyl, tetrahydrofuranyl, oxetanyl, azetidinyl, pyrrolidinyl, morpholinyl, imidazolidinyl, thiomorpholinyl, dioxanyl, piperazinyl and homopiperazinyl. When present, the sulfur atom may be in an oxidized form (i.e., S═O or O═S═O). Exemplary heterocyclic groups containing sulfur in oxidized form are 1,1-dioxido-thiomorpholinyl and 1,1-dioxido-isothiazolidinyl.

The term “heterocyclyl-C₁₋₄-alkyl” denotes a heterocyclic ring as defined above that is directly attached to a straight or branched C₁₋₄-alkyl group via a carbon or nitrogen atom of said ring. Examples of heterocyclyl-C₁₋₄-alkyl groups include piperidin-1-ylmethyl, piperidin-4-ylmethyl and morpholin-4-ylmethyl.

The term “heteroaryl” denotes a monocyclic or fused bicyclic heteroaromatic ring system comprising 5 to 10 ring atoms in which one or more of the ring atoms are other than carbon, such as nitrogen, sulphur or oxygen. Only one ring need to be aromatic and said heteroaryl moiety can be linked to the remainder of the molecule via a carbon or nitrogen atom in any ring. Examples of heteroaryl groups include furyl, pyrrolyl, thienyl, oxazolyl, isoxazolyl, imidazolyl, thiazolyl, isothiazolyl, pyridinyl, pyrimidinyl, tetrazolyl, quinazolinyl, indolyl, indolinyl, isoindolyl, isoindolinyl, pyrazolyl, pyridazinyl, pyrazinyl, quinolinyl, quinoxalinyl, oxadiazolyl, thiadiazolyl, benzofuranyl, 2,3-dihydrobenzofuranyl, 1,3-benzodioxolyl, 1,4-benzodioxinyl, benzothiazolyl, benzimidazolyl, benzotriazolyl and chromanyl.

The term “heteroaryl-C₁₋₄-alkyl” denotes a heteroaryl ring as defined above that is directly linked to a straight or branched C₁₋₄-alkyl group via a carbon or nitrogen atom of said ring. Examples of such groups include 2-(pyridin-2-yl)-ethyl and 1,2,4-oxadiazol-5-ylmethyl.

The term “C₂₋₄-alkenyl” denotes a straight or branched hydrocarbon chain radical having from 2 to 4 carbon atoms and containing one carbon-carbon double bond. Examples of said C₂₋₄-alkenyl include vinyl, allyl, 2-methylallyl and 1-butenyl.

The term “C₁₋₄-alkylene” denotes a straight or branched divalent saturated hydrocarbon chain having from 1 to 4 carbon atoms. Examples of C₁₋₄-alkylene diradicals include methylene [—CH₂—], 1,2-ethylene [—CH₂—CH₂—], 1,1-ethylene [—CH(CH₃)—], 1,2-propylene [—CH₂—CH(CH₃)—] and 1,3-propylene [—CH₂—CH₂—CH₂—]. When referring to e.g. a “C₁₋₄-alkylene” radical, all subgroups thereof are contemplated, such as C₁₋₃-alkylene, C₁₋₂-alkylene, C₂₋₄-alkylene, C₂₋₃-alkylene and C₃₋₄-alkylene.

“Halogen” refers to fluorine, chlorine, bromine or iodine.

“Hydroxy” refers to the —OH radical.

“Cyano” refers to the —CN radical.

“Optional” or “optionally” means that the subsequently described event or circumstance may but need not occur, and that the description includes instances where the event or circumstance occurs and instances in which it does not.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

“Prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2^{nd} Ed., Elsevier Academic Press (2004), pp. 498-549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

Throughout the specification and the appended claims, a given chemical formula or name shall also encompass all salts, hydrates, solvates, N-oxides and prodrug forms thereof. Further, a given chemical formula or name shall encompass all tautomeric and stereoisomeric forms thereof. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in their pure cis or trans forms or as mixtures of those.

The compounds of formula (I) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### Compositions

For clinical use, the compounds of the invention are formulated into pharmaceutical formulations for various modes of administration. It will be appreciated that compounds of the invention may be administered together with a physiologically acceptable carrier, excipient, or diluent. The pharmaceutical compositions of the invention may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the invention may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### Preparation of Compounds of the Invention

The compounds of formula (I) above may be prepared by, or in analogy with, conventional methods. The preparation of intermediates and compounds according to the examples of the present invention may in particular be illuminated by the following Schemes. Definitions of variables in the structures in the schemes herein are commensurate with those of the corresponding positions in the formulae delineated herein.

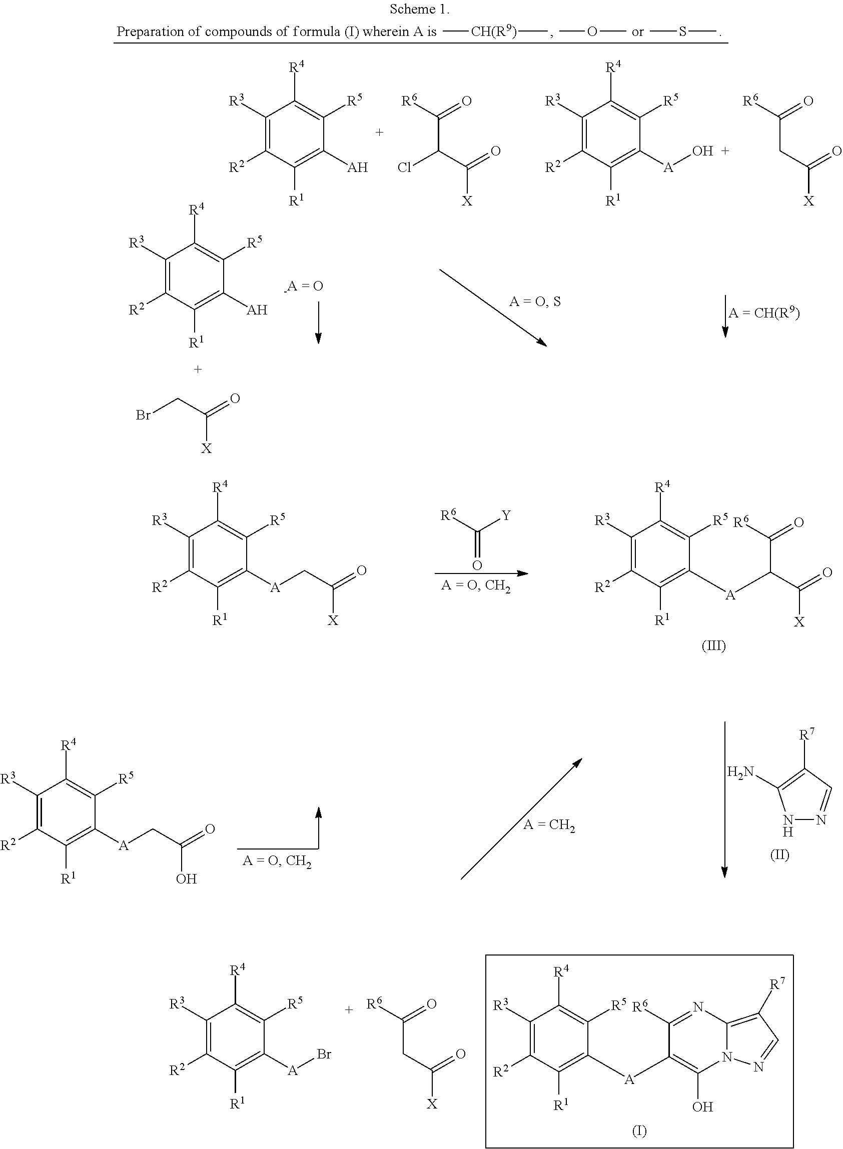

wherein R¹-R⁵, R⁶, R⁷ and R⁹ are as defined in formula (I); and X and Y are each independently —OMe or —OEt.

Compounds of general formula (I) wherein A is —CH(R⁹)—, —O— or —S— can easily be prepared by the condensation of a 3-aminopyrazole derivative of formula (II) with the appropriate α-substituted-β-keto ester of formula (III), as illustrated in Scheme 1 above. Compounds of general formula (I) wherein A is —N(R¹⁰)— can similarly be prepared by the condensation of a 3-aminopyrazole derivative of formula (II) with the appropriate α-substituted-β-imino ester of formula (IV), as shown in Scheme 2 below. The condensation is typically achieved by heating, optionally in the presence of acid or Lewis acid catalysts, including, but not limited to, acetic acid, phosphoric acid, hydrochloric acid, sulfuric acid and titanium trichloride.

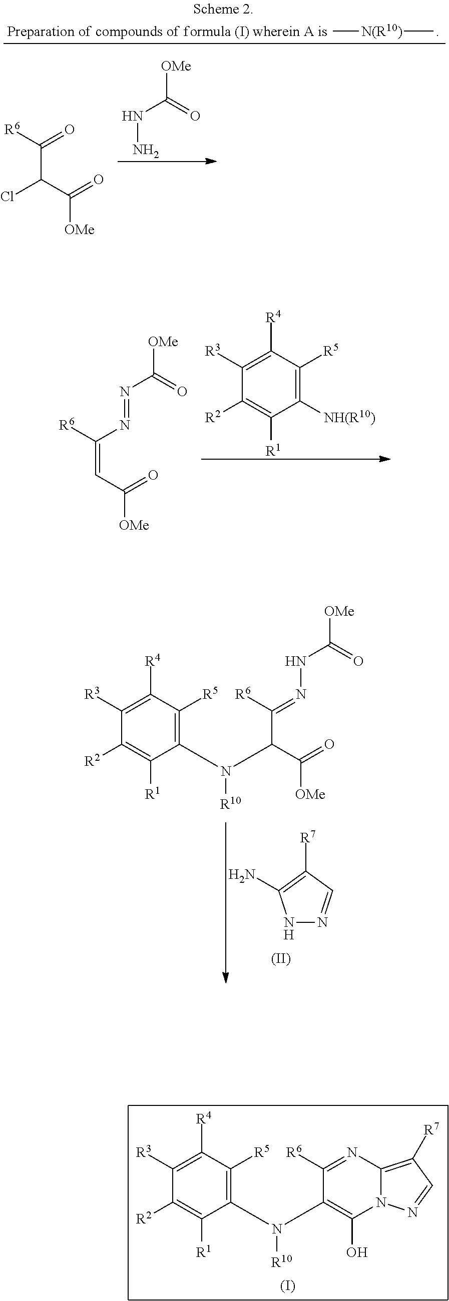

wherein R¹-R⁵, R⁶, R⁷ and R¹⁰ are as defined in formula (I).

The intermediate 3-aminopyrazoles of formula (II), α-substituted-β-keto esters of formula (III) and α-substituted-β-imimo esters of formula (IV) are either commercially available, or may be prepared by methods known in the art. Such methods include, but are not limited to, those illustrated in the Schemes. For example, α-benzyl-β-keto esters of formula (III) (A=—CH₂— or —CH(R⁹)—) may be prepared by condensation of β-keto esters with benzyl alcohols or benzyl bromides, or by condensation of 3-aryl-propionic esters with dialkyloxalates. α-Phenoxy-β-keto esters of formula (III) (A=O) may be prepared by condensation of α-chloro-β-keto esters with phenols, or by condensation of aryloxy-acetates with dialkyloxalates. 3-(Methoxy-carbonyl-hydrazono)-2-arylamino esters of formula (IV) may be prepared by condensation of α-chloro-β-keto esters with methyl carbazate followed by treatment with anilines. All of these alternatives are exemplified in the experimental section below.

Appropriate reaction conditions for the individual reaction steps are known to a person skilled in the art. Particular reaction conditions for examples of the invention are also described in the experimental section. The necessary starting materials for preparing the compounds of formula (I) are either commercially available, or may be prepared by methods known in the art.

The processes described below in the experimental section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl (triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The following abbreviations have been used:

AcOH Acetic acid
aq aqueous
cBu cyclobutyl
cPr cyclopropyl
DBN 1,5-Diazabicyclo[4.3.0]non-5-ene
DBU 1,8-Diazabicyclo(5.4.0)undec-7-ene

### DCM Dichloromethane

### DIPEA N,N-Diisopropylethylamine

### DMF N,N-Dimethylformamide

EDC 1-Ethyl-3-(3-dimethylaminopropyl)carbodiimide
ESI⁺ Electrospray ionization
Et₂O Diethyl ether
EtOAc Ethyl acetate

### EtOH Ethanol

HBTU 2-(1H-Benzotriazole-1-yl)-1,1,3,3-tetramethyluronium hexafluorophosphate

### HOBt N-Hydroxybenzotriazole

HONB endo-N-Hydroxybicyclo[2.2.1]hept-5-ene-2,3-dicarboximide

### HPLC High Performance Liquid Chromatography

### HPLC-MS High Performance Liquid Chromatography-Mass Spectrometry

### HRMS High Resolution Mass Spectrometry

### M Molar

### MeCN Acetonitrile

### MeOH Methanol

[MH]⁺ Protonated molecular ion

### LCMS Liquid Chromatography Mass Spectrometry

sat saturated
T₃P 2-Propane phosphinic acid anhydride
TFA Trifluoroacetic acid

### THF Tetrahydrofuran

### TLC Thin Layer Chromatography

### TMEDA Tetramethylethylenediamine

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

### EXAMPLES AND INTERMEDIATE COMPOUNDS

### Experimental Methods

All reagents were commercial grade and were used as received without further purification, unless otherwise specified. Reagent grade solvents were used in all cases. Analytical HPLC-MS was performed on an Agilent 1100 system equipped with an ACE C8, 30×3.0 mm, 3 μm column (MeCN/MeOH (95/5) in water (5 mM ammonium acetate), 215-295 nm, 35° C.). High-resolution mass spectra (HRMS) were obtained on an Agilent MSD-TOF connected to an Agilent 1100 HPLC system. During the analyses the calibration was checked by two masses and automatically corrected when needed. Spectra are acquired in positive electrospray mode. The acquired mass range was m/z 100-1100. Profile detection of the mass peaks was used. Analytical HPLC was performed on either an Agilent 1100 system using a Phenomenex Synergi, RP-Hydro, 150×4.6 mm, 4 μm column with a flow rate of 1.5 mL per min at 30° C. and a gradient of 5-100% acetonitrile (+0.085% TFA) in water (+0.1% TFA) over 7 min, (200-300 nm), or on an Agilent 1100/1200 Series Liquid chromatograph/Mass Selective Detector (Single Quadrupole) equipped with an electrospray interface using a gradient of 5-100% acetonitrile in water (5 mM ammonium acetate) over 4 min, 1 mL/min, 215-395 nM (marked * in text below), unless otherwise stated. Figures quoted are column retention time and % purity. Flash chromatography was performed on either a CombiFlash Companion system equipped with RediSep silica columns or a Flash Master Personal system equipped with Strata SI-1 silica gigatubes or in a glass column under gravity. Reverse Phase HPLC was performed on a Gilson system (Gilson 322 pump with Gilson 321 equilibration pump and Gilson 215 autosampler) equipped with Phenomenex Synergi Hydro RP 150×10 mm, or YMC ODS-A 100/150×20 mm columns, or on an XTerra Prep MS C18 5 μm 19×50 mm system. Reverse phase column chromatography was performed on a Gilson system (Gilson 321 pump and Gilson FC204 fraction collector) equipped with Merck LiChroprep® RP-18 (40-63 um) silica columns. Microwave irradiations were carried out using a Biotage microwave. The compounds were automatically named using ACD 6.0. All compounds were dried in a vacuum oven overnight. Where yields are not included, the intermediates were used crude. Reactions were monitored by TLC, LCMS or HPLC.

### Intermediate 1

### General Procedure A

### Ethyl 2-[(4-chlorophenyl)methyl]-3-oxobutanoate

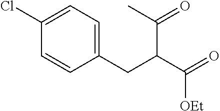 [SMILES: CCOC(=O)C(Cc1ccc(Cl)cc1)C(C)=O]

Ethyl acetoacetate (10.0 g, 76.8 mmol) was dissolved in DMF (160 mL) and 4-chlorobenzyl bromide (15.0 g, 73.0 mmol) and lithium carbonate (5.68 g, 76.8 mmol) were added. The reaction was heated at 80° C. for 48 h. The reaction mixture was diluted with water (100 mL) and toluene (200 mL) and the organic phase was washed with water (3×100 mL), brine (100 mL), dried (MgSO₄), and the solvents removed in vacuo to give crude title compound (18.2 g) which was used without further purification or characterization.

### Intermediate 2

### General Procedure B

### Ethyl 2-[(3,4-dichlorophenyl)methyl]-3-oxobutanoate

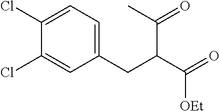 [SMILES: CCOC(=O)C(Cc1ccc(Cl)c(Cl)c1)C(C)=O]

Ethyl acetoacetate (500 mg, 3.84 mmol) was dissolved in toluene (20 mL) and 3,4-dichloro-benzyl bromide (920 mg, 3.84 mmol) and potassium carbonate (377 mg, 2.73 mmol) were added. The reaction was heated at reflux for 15 h. The reaction mixture was diluted with water (30 mL) and toluene (50 mL) and the organic phase was washed with water (3×20 mL), brine (50 mL), dried (Na₂SO₄) and the solvents removed in vacuo. The residue was purified by column chromatography to give crude title compound (500 mg) as a pale brown liquid which was used without further purification or characterization.

### Intermediate 3

### General Procedure C

### Methyl 2-[(2,3-difluorophenyl)methyl]-3-oxobutanoate

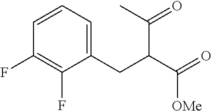 [SMILES: COC(=O)C(Cc1cccc(F)c1F)C(C)=O]

Sodium hydride (192 mg, 60% dispersion in mineral oil, 4.80 mmol) was suspended in THF (20 mL) at room temperature and methyl acetoacetate (296 μL, 2.74 mmol) was added dropwise. 2,3-Difluorobenzyl bromide (383 μL, 3.01 mmol) was added and the reaction mixture was stirred for 16 h. The reaction mixture was quenched with sat aq NH₄Cl (5 mL) and water (25 mL) and extracted with EtOAc (3×50 mL). The combined organic layers were dried (MgSO₄) and concentrated in vacuo to give crude title compound which was used without further purification or characterization.

### Intermediate 4

### Methyl 2-acetyl-3-(4-chlorophenyl)butanoate

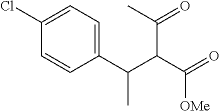 [SMILES: COC(=O)C(C(C)=O)C(C)c1ccc(Cl)cc1]

Methyl acetoacetate (465 μL, 4.31 mmol), 1-(4-chloro-phenyl)-ethanol (0.67 g, 4.31 mmol) and FeCl₃ (69.8 mg, 0.43 mmol) were dissolved in DCM (15 mL) and heated at reflux for 16 h. The reaction mixture was filtered through celite and the solvents were removed in vacuo to give crude title compound (971 mg) as a brown liquid which used without further purification or characterization.

### Intermediates 5-59

Intermediates 5-59 were prepared similarly to General Procedures A-C, by reacting beta-keto esters with the appropriate benzyl bromides (0.9-1.2 eq) at 20-50° C. for 5-72 h; see Table 1 below.

### Intermediate 60

### General Procedure D

### Methyl 2-(3-fluorophenoxy)-3-oxobutanoate

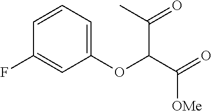 [SMILES: COC(=O)C(Oc1cccc(F)c1)C(C)=O]

Sodium hydride (221 mg, 60% dispersion in mineral oil, 3.32 mmol) was suspended in THF (2 mL) at 0° C. and a solution of 3-fluorophenol (372 mg, 3.32 mmol) in THF (2 mL) was added. The reaction mixture was warmed to room temperature over 1 h. TMEDA (500 μL, 3.32 mmol) and methyl-2-chloroacetoacetate (500 mg, 3.32 mmol) were added and the reaction mixture was heated at reflux for 4 h. The solvents were removed in vacuo and the residue was partitioned between DCM (15 mL) and 1 M aq NaOH (2 mL). The organic fraction was washed with water (5 mL), dried (MgSO₄) and concentrated in vacuo. The residue was purified by column chromatography to give the crude title compound (195 mg, 26%) as a yellow oil which was used without further purification or characterization.

### Intermediates 61-69

Intermediates 61-69 were prepared similarly to General Procedure D; see Table 2 below.

TABLE 1
Preparation of beta-keto ester intermediates

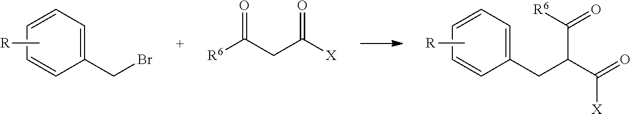

| Int | R | R⁶ | X | Proc | Intermediate Name |
|---|---|---|---|---|---|
| 5 | 4-Me | Me | OEt | A | Ethyl 2-[(4-methylphenyl)methyl]-3-oxobutanoate |
| 6 | 3-Cl | Me | OEt | B | Ethyl 2-[(3-chlorophenyl)methyl]-3-oxobutanoate |
| 7 | 4-CF₃ | Me | OEt | A | Ethyl 3-oxo-2-{[4-(trifluoromethyl)phenyl]methyl}butanoate |
| 8 | 3-CN, 4-F | Me | OMe | C | Methyl 2-[(3-cyano-4-fluorophenyl)methyl]-3-oxotutanoate |
| 9 | 3-Br, 4-Cl | Me | OMe | C | Methyl 2-[(3-bromo-4-chlorophenyl)methyl]-3-oxobutanoate |
| 10 | 3-OMe | Me | OMe | C | Methyl 2-[(3-methoxyphenyl)methyl]-3-oxobutanoate |
| 11 | 4-Cl | Et | OEt | C | Ethyl 2-[(4-chlorophenyl)methyl]-3-oxopentanoate |
| 12 | 3,5-F₂ | Et | OEt | C | Ethyl 2-[(3,5-difluorophenyl)methyl]-3-oxopentanoate |
| 13 | 3,4,5-F₃ | Et | OEt | C | Ethyl 3-oxo-2-[(3,4,5-trifluorophenyl)methyl]pentanoate |
| 14 | 3-Cl, 4-F | Et | OEt | C | Ethyl 2-[(3-chloro-4-fluorophenyl)methyl]-3-oxopentanoate |
| 15 | 3-F, 5-CF₃ | Et | OEt | C | Ethyl 2-{[3-fluoro-5-(trifluoromethyl)phenyl]methyl}-3-oxopantanoate |
| 16 | 3,4-Cl₂ | Et | OEt | C | Ethyl 2-[(3,4-dichlorophenyl)methyl]-3-oxopentanoate |
| 17 | 3-F, 4-CF₃ | ⁿPr | OEt | C | Ethyl 2-{[3-fluoro-4-(trifluoromethyl)phenyl]methyl}-3-oxohexanoate |
| 18 | 3,4-Cl₂ | ⁿPr | OEt | C | Ethyl 2-[(3,4-dichlorophenyl)methyl]-3-oxohexanoate |
| 19 | H | ^{c}Pr | OEt | C | Ethyl 2-benzyl-3-cyclopropyl-3-oxopropanoate |
| 20 | 4-CF₃O | ^{c}Pr | OEt | C | Ethyl 3-cyclopropyl-3-oxo-2-{[4-(trifluoromethoxy)phenyl]methyl}propanoate |
| 21 | 3-CF₃O | ^{c}Pr | OEt | C | Ethyl 3-cyclopropyl-3-oxo-2-{[3-(trifluoromethoxy)phenyl]methyl}propanoate |
| 22 | 3-Cl | ^{c}Pr | OEt | C | Ethyl 2-[(3-chlorophenyl)methyl]-3-cyclopropyl-3-oxopropanoate |
| 23 | 3-CF₃ | ^{c}Pr | OEt | C | Ethyl 3-cyclopropyl-3-oxo-2-{[3-(trifluoromethyl)phenyl]methyl}propanoate |
| 24 | 4-CF₃ | ^{c}Pr | OEt | C | Ethyl 3-cyclopropyl-3-oxo-2-{[4-(trifluoromethyl)phenyl]methyl}propanoate |
| 25 | 3,4-F₂ | ^{c}Pr | OEt | C | Ethyl 3-cyclopropyl-2-[(3,4-difluorophenyl)methyl]-3-oxopropanoate |
| 26 | 3,5-F₂ | ^{c}Pr | OEt | C | Ethyl 3-cyclopropyl-2-[(3,5-difluorophenyl)methyl]-3-oxopropanoate |
| 27 | 3,4,5-F₃ | ^{c}Pr | OEt | C | Ethyl 3-cyclopropyl-3-oxo-2-[(3,4,5-trifluorophenyl)methyl]propanoate |
| 28 | 3-F, 4-Cl | ^{c}Pr | OEt | C | Ethyl 2-[(4-chloro-3-fluorophenyl)methyl]-3-cyclopropyl-3-oxopropanoate |
| 29 | 3-Cl, 4-F | ^{c}Pr | OEt | C | Ethyl 2-[(3-chloro-4-fluorophenyl)methyl]-3-cyclopropyl-3-oxopropanoate |
| 30 | 3-Cl, 5-F | ^{c}Pr | OEt | C | Ethyl 2-[(3-chloro-5-fluorophenyl)methyl]-3-cyclopropyl-3-oxopropanoate |
| 31 | 3,4-Cl₂ | ^{c}Pr | OEt | C | Ethyl 3-cylcopropyl-2-[(3,4-dichlorophenyl)methyl]-3-oxopropanoate |
| 32 | 3-CF₃, 4-F | ^{c}Pr | OEt | C | Ethyl 3-cyclopropyl-2-{[4-fluoro-3-(trifluoromethyl)phenyl]methyl}-3- |
| | | | | | oxopropanoate |
| 33 | 3-F, 4-CF₃ | ^{c}Pr | OEt | C | Ethyl 3-cyclopropyl-2-{[3-fluoro-4-(trifluoromethyl)phenyl]methyl}-3- |
| | | | | | oxopropanoate |
| 34 | 3-F, 5-CF₃ | ^{c}Pr | OEt | C | Ethyl 3-cyclopropyl-2-{[3-fluoro-5-(trifluoromethyl)phenyl]methyl}-3- |
| | | | | | oxopropanoate |
| 35 | 3,5-Cl₂ | ^{c}Pr | OEt | C | Ethyl 3-cyclopropyl-2-[(3,5-dichlorophenyl)methyl]-3-oxopropanoate |
| 36 | 3,4-F₂ | ⁱPr | OEt | C | Ethyl 2-[(3,4-difluorophenyl)methyl]-4-methyl-3-oxopentanoate |
| 37 | 3,4,5-F₃ | ⁱPr | OEt | C | Ethyl 4-methyl-3-oxo-2-[(3,4,5-trifluorophenyl)methyl]pentanoate |
| 38 | 3-Cl, 4-F | ⁱPr | OEt | C | Ethyl 2-[(3-chloro-4-fluorophenyl)methyl]-4-methyl-3-oxopentanoate |
| 39 | 3-F, 4-Cl | ⁱPr | OEt | C | Ethyl 2-[(4-chloro-3-fluorophenyl)methyl]-4-methyl-3-oxopentanoate |
| 40 | 3-Cl, 5-F | ⁱPr | OEt | C | Ethyl 2-[(3-chloro-5-fluorophenyl)methyl]-4-methyl-3-oxopentanoate |
| 41 | 3-CF₃, 4-F | ⁱPr | OEt | C | Ethyl 2-{[4-fluoro-3-(trifluoromethyl)phenyl]methyl}-4-methyl-3-oxopentanoate |
| 42 | 3,4-Cl₂ | ⁱPr | OEt | C | Ethyl 2-[(3,4-dichlorophenyl)methyl]-4-methyl-3-oxopentanoate |
| 43 | 3,5-Cl₂ | ⁱPr | OEt | C | Ethyl 2-[(3,5-dichlorophenyl)methyl]-4-methyl-3-oxopentanoate |
| 44 | 3-Cl | ^{c}Bu | OEt | C | Ethyl 2-[(3-chlorophenyl)methyl]-3-cyclobutyl-3-oxopropanoate |
| 45 | 4-Cl | ^{c}Bu | OEt | C | Ethyl 2-[(4-chlorophenyl)methyl]-3-cyclobutyl-3-oxopropanoate |
| 46 | 3,4-F₂ | ^{c}Bu | OEt | C | Ethyl 3-cyclobutyl-2-[(3,4-difluorophenyl)methyl]-3-oxopropanoate |
| 47 | 3-Cl, 4-F | ^{c}Bu | OEt | C | Ethyl 2-[(3-chloro-4-fluorophenyl)methyl]-3-cyclobutyl-3-oxopropanoate |
| 48 | 3-CF₃, 4-F | ^{c}Bu | OEt | C | Ethyl 3-cyclobutyl-2-{[4-fluoro-3-(trifluoromethyl)phenyl]methyl}-3- |
| | | | | | oxopropanoate |
| 49 | 3-F, 5-CF₃ | ^{c}Bu | OEt | C | Ethyl 3-cyclobutyl-2-{[3-fluoro-5-(trifluoromethyl)phenyl]methyl}-3- |
| | | | | | oxopropanoate |
| 50 | 3,4,5-F₃ | ^{c}Bu | OEt | C | Ethyl 3-cyclobutyl-3-oxo-2-[(3,4,5-trifluorophenyl)methyl]propanoate |
| 51 | 3,4-Cl₂ | ^{c}Bu | OEt | C | Ethyl 3-cyclobutyl-2-[(3,4-dichlorophenyl)methyl]-3-oxopropanoate |
| 52 | 4-Cl | CH₂OMe | OMe | C | Methyl 2-[(4-chlorophenyl)methyl]-4-methoxy-3-oxobutanoate |
| 53 | 3-F, 4-Cl | CH₂OMe | OMe | C | Methyl 2-[(4-chloro-3-fluorophenyl)methyl]-4-methoxy-3-oxobutanoate |
| 54 | 3,4-Cl₂ | CH₂OMe | OMe | C | Methyl 2-[(3,4-dichlorophenyl)methyl]-4-methoxy-3-oxobutanoate |
| 55 | 4-Cl | (CH₂)₂OMe | OMe | C | Methyl 2-[(4-chlorophenyl)methyl]-5-methoxy-3-oxopentanoate |
| 56 | 3,4-Cl₂ | CF₃ | OEt | C | Ethyl 2-[(3,4-dichlorophenyl)methyl]-4,4,4-trifluoro-3-oxobutanoate |
| 57 | 3,4-Cl₂ | THF-2-yl | OEt | C | Ethyl 2-[(3,4-dichlorophenyl)methyl]-3-oxo-3-(oxolan-2-yl)propanoate |
| 58 | 4-Cl | CH₂OBn | OMe | C(Int 99) | Methyl 4-(benzyloxy)-2-[(4-chlorophenyl)methyl]-3-oxobutanoate |
| 59 | 3-F | Pyrazin-2-yl- | OEt | C | Ethyl 2-[(3-fluorophenyl)methyl]-3-oxo-3-(pyrazin-2-yl)propanoate |

TABLE 2
Preparation of beta-keto ester intermediates

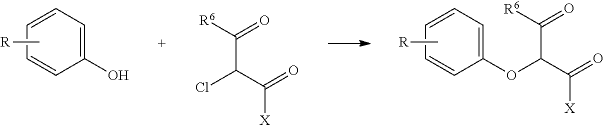

| Int | R | R⁶ | X | Intermediate Name |
|---|---|---|---|---|
| 61 | 3,4-Cl₂ | Me | OMe | Methyl 2-(3,4-dichlorophenoxy)-3-oxobutanoate |
| 62 | 3-CF₃ | Et | OMe | Methyl 3-oxo-2-[3-(trifluoromethyl)phenoxy]pentanoate |
| 63 | 3,4-F₂ | Et | OMe | Methyl 2-(3,4-difluorophenoxy)-3-oxopentanoate |
| 64 | 3-Cl, 4-F | Et | OEt | Ethyl 2-(3-chloro-4-fluorophenoxy)-3-oxopentanoate |
| 65 | 3,4-Cl₂ | Et | OMe | Methyl 2-(3,4-dichlorophenoxy)-3-oxopentanoate |
| 66 | 4-Br | ^{c}Pr | OEt | Ethyl 2-(4-bromophenoxy)-3-cyclopropyl-3-oxopropanoate |
| 67 | 4-Br | ⁱPr | OEt | Ethyl 2-(4-bromophenoxy)-4-methyl-3-oxopentanoate |
| 68 | 3-F, 4-Cl | ⁱPr | OMe | Methyl 2-(4-chloro-3-fluorophenoxy)-4-methyl-3-oxopentanoate |
| 69 | 3,4-Cl₂ | ⁱPr | OMe | Methyl 2-(3,4-dichlorophenoxy)-4-methyl-3-oxopentanoate |

### Intermediate 70

### General Procedure E

### Methyl 3-(3-fluorophenyl)propanoate

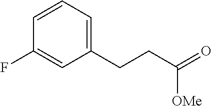 [SMILES: COC(=O)CCc1cccc(F)c1]

3-(3-Fluorophenyl) propionic acid (5.00 g, 29.7 mmol) was dissolved in MeOH (50 mL) and H₂SO₄ (1 mL) was added. The reaction mixture was heated at reflux for 6 h and concentrated in vacuo to approximately 15 mL. EtOAc (100 mL) was added and the organic fraction was washed with 1 M aq Na₂CO₃ (2×100 mL), dried (MgSO₄) and the solvents were removed in vacuo to give the title compound as a pale yellow oil (5.28 g, 98%) which used without further purification or characterization.

### Intermediates 71-77

Intermediates 71-77 were prepared similarly to General Procedure E; see Table 3 below.

TABLE 3
Esterification of 3-aryl propionic acids

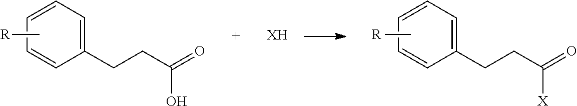

| Int | R | X | Yield | Intermediate Name |
|---|---|---|---|---|
| 71 | 3-Cl | OEt | 97% | Ethyl 3-(3-chlorophenyl)propanoate |
| 72 | 3-CF₃ | OEt | n/a | Ethyl 3-[3-(trifluoromethyl)phenyl]propanoate |
| 73 | 3-Cl, 4-F | OEt | 97% | Ethyl 3-(3-chloro-4-fluorophenyl)propanoate |
| 74 | 2,4-Cl₂ | OEt | 93% | Ethyl 3-(2,4-dichlorophenyl)propanoate |
| 75 | 3,4-Cl₂ | OEt | 100% | Ethyl 3-(3,4-dichlorophenyl)propanoate |
| 76 | 3-F | OEt | 96% | Ethyl 3-(3-fluorophenyl)propanoate |
| 77 | 3-F, 4-Cl | OEt | 97% | Ethyl 3-(4-chloro-3-fluorophenyl)propanoate |

### Intermediate 78

### General Procedure F

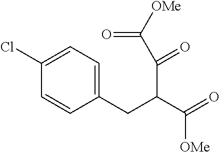 [SMILES: COC(=O)C(=O)C(Cc1ccc(Cl)cc1)C(=O)OC]

Dimethyl oxalate (1.18 g, 10.0 mmol) was dissolved in Et₂O (20 mL) and added to sodium hydride (400 mg, 60% dispersion in mineral oil, 10.0 mmol). MeOH (2 drops) was added and the reaction mixture was heated to 50° C. A solution of 3-(4-chlorophenyl)propionic acid methyl ester (1.99 g, 10.0 mmol) in Et₂O (20 mL) was added dropwise and the reaction mixture was heated at reflux for 2 d. The precipitate was collected by filtration, dissolved in water (50 mL) and acidified to pH 1 with 1 M aq HCl (50 mL). The reaction mixture was extracted with Et₂O (3×100 mL) and the combined organic fractions were washed with water (2×100 mL), dried (Na₂SO₄) and concentrated in vacuo to give the title compound (700 mg, 25%) as an orange oil which used without further purification or characterization.

### Intermediate 79

### General Procedure G

### 1,4-Diethyl 2-[(3-chlorophenyl)methyl]-3-oxobutanedioate

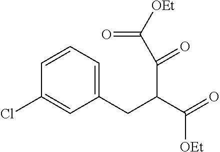 [SMILES: CCOC(=O)C(=O)C(Cc1cccc(Cl)c1)C(=O)OCC]

Sodium hydride (995 mg, 60% dispersion in mineral oil, 24.9 mmol) was suspended in THF (100 mL) and diethyl oxalate (3.76 mL, 24.9 mmol), Intermediate 71 (4.81 g, 22.6 mmol) and EtOH (400 μL) were added. The reaction mixture was heated at reflux for 2 h and was quenched with sat aq NH₄Cl (10 mL) and water (150 mL) and extracted with EtOAc (3×150 mL). The combined organic layers were dried (MgSO₄) and concentrated in vacuo to give the title compound (6.93 g, 98%) as a yellow oil which used without further purification or characterization.

### Intermediates 80-86

Intermediates 80-86 were prepared similarly to General Procedures F-G; see Table 4 below.

TABLE 4
Preparation of beta-keto ester intermediates

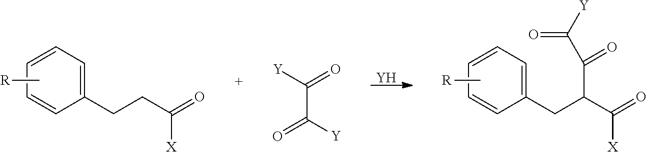

| | | | | | SM | | |
|---|---|---|---|---|---|---|---|
| Int | R | X | Y | Proc | (Int) | Yield | Intermediate Name |
| 80 | 3-F | OMe | OMe | F | 71 | 67% | 1,4-Dimethyl 2-[(3-fluorophenyl)methyl]-3-oxobutanedioate |
| 81 | 3-CF₃ | OEt | OEt | G | 72 | n/a | 1,4-Diethyl 2-oxo-3-{[3-(trifluoromethyl)phenyl]methyl}butanedioate |
| 82 | 3-Cl, 4-F | OEt | OEt | G | 73 | 59% | 1,4-Diethyl 2-[(3-chloro-4-fluorophenyl)methyl]-3-oxobutanedioate |
| 83 | 2,4-Cl₂ | OEt | OEt | G | 74 | 97% | 1,4-Diethyl 2-[(2,4-dichlorophenyl)methyl]-3-oxobutanedioate |
| 84 | 3,4-Cl₂ | OEt | OEt | G | 75 | 96% | 1,4-Diethyl 2-[(3,4-dichlorophenyl)methyl]-3-oxobutanedioate |
| 85 | 3-F | OEt | OEt | G | 76 | 93% | 1,4-Diethyl 2-[(3-fluorophenyl)methyl]-3-oxobutanedioate |
| 86 | 3-F, 4-Cl | OEt | OEt | G | 77 | 57% | 1,4-Diethyl 2-[(4-chloro-3-fluorophenyl)methyl]-3-oxobutanedioate |

### Intermediate 87

### Ethyl 2-(4-chloro-3-fluorophenoxy)acetate

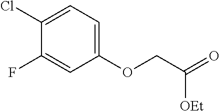 [SMILES: CCOC(=O)COc1ccc(Cl)c(F)c1]

Sodium hydride (600 mg, 60% dispersion in mineral oil, 15.0 mmol) was suspended in THF (50 mL) and 4-chloro-3-fluorophenol (2.00 g, 13.7 mmol) and ethylbromoacetate (1.51 mL, 13.7 mmol) were added. The reaction mixture was stirred for 18 h, diluted with EtOAc (200 mL), washed with 1M aq NaOH (3×100 mL) and water (100 mL), dried (MgSO₄) and concentrated in vacuo to give the title compound as a pale yellow oil (2.20 g, 69%) which was used without further purification or characterization.

### Intermediate 88

### Ethyl 2-(4-chloro-3-fluorophenoxy)acetate

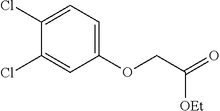 [SMILES: CCOC(=O)COc1ccc(Cl)c(Cl)c1]

The title compound (3.64 g, 95%) was prepared similarly to Intermediate 87, using 3,4-dichlorophenol instead of 4-chloro-3-fluorophenol.

### Intermediate 89

### 1,4-Diethyl 2-(4-chloro-3-fluorophenoxy)-3-oxobutanedioate

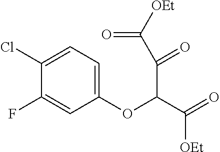 [SMILES: CCOC(=O)C(=O)C(Oc1ccc(Cl)c(F)c1)C(=O)OCC]

Sodium hydride (416 mg, 60% dispersion in mineral oil, 10.4 mmol) was suspended in THF (50 mL) and diethyl oxalate (1.41 mL, 10.4 mmol), Intermediate 87 (2.20 g, 9.46 mmol) and EtOH (200 μL) were added. The reaction mixture was heated at reflux for 3 h, quenched with sat NH₄Cl (10 mL) and water (250 mL) and extracted with EtOAc (3×250 mL). The combined organic fractions were dried (MgSO₄) and the solvents were removed in vacuo to give the crude title compound (2.45 g, 78%) as an orange oil which was used without further purification or characterization.

### Intermediate 90

### 1,4-Diethyl 2-(3,4-dichlorophenoxy)-3-oxobutanedioate

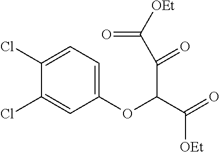 [SMILES: CCOC(=O)C(=O)C(Oc1ccc(Cl)c(Cl)c1)C(=O)OCC]

The title compound (5.03 g, 99%) was prepared similarly to Intermediate 89, using Intermediate 88 instead of Intermediate 87.

### Intermediate 91

### 6-[(3-Fluorophenyl)sulfanyl]-5-methylpyrazolo[1,5-a]pyrimidin-7-ol

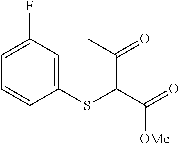 [SMILES: COC(=O)C(Sc1cccc(F)c1)C(C)=O]

Methyl 2-chloroacetoacetate (501 mg, 3.32 mmol) and 3-fluorothiophenol (355 μL, 3.32 mmol) were dissolved in DCM (5 mL) and cooled to 0° C. A solution of triethylamine (508 μL, 3.65 mmol) in DCM (1.5 mL) was added dropwise and the reaction mixture was warmed to room temperature. The reaction mixture was diluted with hexanes (30 mL), washed with water (2×15 mL) and brine (15 mL), dried (MgSO₄) and concentrated in vacuo. The residue was purified by column chromatography to give crude title compound (611 mg, 76%) as a yellow liquid which was used without further purification or characterization.

### Intermediate 92

### Methyl 3-[[(methoxycarbonyl)imino]amino]but-2-enoate

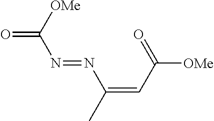

Methyl 2-chloroacetoacetate (1.01 g, 6.71 mmol) was dissolved in Et₂O (10 mL) and methyl carbazate (605 mg, 6.72 mmol) was added. The reaction mixture was stirred for 4 h and the solvents were removed in vacuo to give crude methyl 3-{[(methoxycarbonyl)amino]imino}-2-methylbutanoate (1.48 g) as a yellow solid. This material was suspended in Et₂O (15 mL) and 1 M aq NaHCO₃ (11 mL) was added. The reaction mixture was stirred for 2.5 h and the organic layer was separated and washed with water (20 mL). The combined aqueous fractions were extracted with Et₂O (25 mL) and the combined organic fractions were dried (MgSO₄) and concentrated in vacuo to give crude title compound (0.96 g) as a red liquid.

### Intermediate 93

### General Procedure H

### Methyl 2-[(3-fluorophenyl)amino]-3-{[(methoxycarbonyl)amino]imino}butanoate

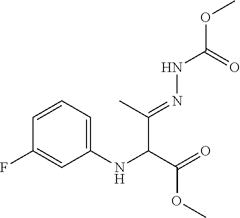 [SMILES: COC(=O)N/N=C(\C)C(Nc1cccc(F)c1)C(=O)OC]

Intermediate 92 (0.48 g, 2.58 mmol) was dissolved in THF (2.5 mL) and a solution of 3-fluoroaniline (326 mg, 2.93 mmol) in THF (2.5 mL) was added. The reaction mixture was stirred at room temperature for 16 h. The solvent was removed in vacuo and the residue was triturated from hexanes to give crude title compound (638 mg) as a yellow solid which was used without further purification or characterization.

### Intermediate 94

### Methyl 2-[(3-fluorophenyl)(methyl)amino]-3-{[(methoxycarbonyl)amino]imino}butanoate

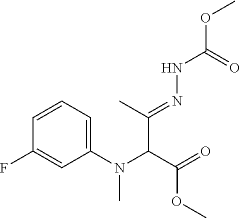 [SMILES: COC(=O)N/N=C(\C)C(C(=O)OC)N(C)c1cccc(F)c1]

Intermediate 92 (0.48 g, 2.58 mmol) and N-methyl-3-fluoroaniline were reacted according to General Procedure H to give the crude title compound (899 mg) as an orange liquid which was used without further purification or characterization.

### Intermediate 95

### Methyl 2-[ethyl(3-fluorophenyl)amino]-3-{[(methoxycarbonyl)amino]imino}butanoate

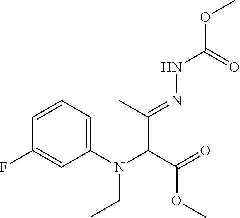 [SMILES: CCN(c1cccc(F)c1)C(C(=O)OC)/C(C)=N/NC(=O)OC]

Intermediate 92 (0.48 g, 2.58 mmol) and N-ethyl-3-fluoroaniline were reacted according to General Procedure H to give the crude title compound (694 mg) which was used without further purification or characterization.

### Intermediate 96

### 7-Hydroxy-6-{[3-(trifluoromethyl)phenyl]methyl}pyrazolo[1,5-a]pyrimidine-5-carboxylic acid

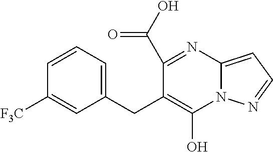 [SMILES: O=C(O)c1nc2ccnn2c(O)c1Cc1cccc(C(F)(F)F)c1]

Example 71 (0.40 g, 1.09 mmol) was suspended in 1 M aq NaOH (10 mL) and heated at reflux for 1 h. The reaction mixture was cooled and acidified with concentrated HCl. The precipitate was collected by filtration and dried to give the title compound (325 mg, 88%) as a beige solid which used without further purification or characterization.

### Intermediate 97

### 6-[(3-Fluorophenyl)methyl]-7-hydroxypyrazolo[1,5-a]pyrimidine-5-carboxylic acid

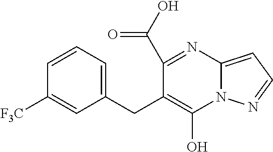 [SMILES: O=C(O)c1nc2ccnn2c(O)c1Cc1cccc(C(F)(F)F)c1]

Example 73 (10.4 g, 33.0 mmol) was dissolved in THF (250 mL) and a solution of LiOH.H₂O (5.54 g, 132 mmol) in water (50 mL) was added. The reaction mixture was stirred for 18 h, acidified with 1 M aq HCl and concentrated in vacuo to approximately 50 mL. The precipitate was collected by filtration to give the title compound as a cream solid (9.46 g, 92%).

### Intermediate 98

### 6-[(3,4-Dichlorophenyl)methyl]7-hydroxypyrazolo[1,5-a]pyrimidine-1-carboxylic acid

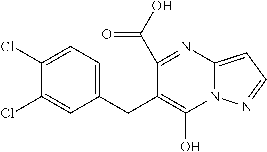 [SMILES: O=C(O)c1nc2ccnn2c(O)c1Cc1ccc(Cl)c(Cl)c1]

The title compound (288 mg, 100%) was prepared similarly to Intermediate 97, using Example 72 instead of Example 73, as a cream solid.

### Intermediate 99

### 6-[(3-Chloro-4-fluorophenyl)methyl]-7-hydroxypyrazolo[1,5-a]pyrimidine-5-carboxylic acid

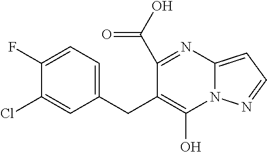 [SMILES: O=C(O)c1nc2ccnn2c(O)c1Cc1ccc(F)c(Cl)c1]

Intermediate 82 (469 mg, 1.42 mmol) and 3-aminopyrazole (130 mg, 1.56 mmol) were dissolved in AcOH (6 mL) and heated at 90° C. for 6 h. The precipitate was collected by filtration, washed with EtOH and dried to give the title compound (73.0 mg, 17%) as a white solid which used without further purification or characterization.

### Intermediate 100

### 6-[(4-Chloro-3-fluorophenyl)methyl]-7-hydroxypyrazolo[1,5-a]pyrimidine-5-carboxylic acid

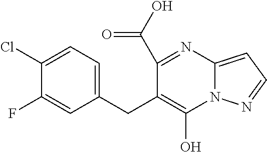 [SMILES: O=C(O)c1nc2ccnn2c(O)c1Cc1ccc(Cl)c(F)c1]

The title compound (33.0 mg, 8%) was prepared similarly to Intermediate 99, using Intermediate 86 instead of Intermediate 82.

### Intermediate 101

### 4-Benzyloxy-3-oxo butyric acid methyl ester

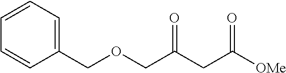 [SMILES: COC(=O)CC(=O)COCc1ccccc1]

Sodium hydride (960 mg, 60% dispersion in mineral oil, 24.0 mmol) was suspended in THF (10 mL) and a solution of benzyl alcohol (1.24 mL, 12.0 mmol) in THF (10 mL) was added dropwise. The reaction mixture was stirred for 30 min and a solution of methyl 4-chloroacetoacetate (1.41 mL, 12.0 mmol) in THF (10 mL) was added dropwise. The reaction mixture was stirred for 16 h, quenched with 2 M aq HCl (25 mL) at 0° C. and adjusted to pH 6. The aqueous phase was extracted with Et₂O (3×50 mL) and the combined organic fractions were washed with sat aq NaHCO₃ (25 mL) and water (50 mL), dried (MgSO₄) and concentrated in vacuo. The residue was purified by column chromatography to give the title compound (1.94 g, 73%) as a pale yellow liquid which used without further purification or characterization.

### Intermediate 102

### 5-[(Benzyloxy)methyl]-6-[(4-chlorophenyl)methyl]pyrazolo[1,5-a]pyrimidin-7-ol

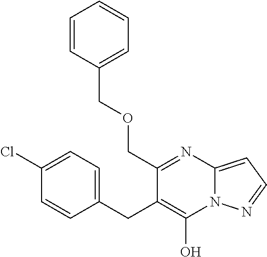 [SMILES: Oc1c(Cc2ccc(Cl)cc2)c(COCc2ccccc2)nc2ccnn12]

Intermediate 58 (1.23 g, 3.54 mmol) and 3-aminopyrazole (309 mg, 3.72 mmol) were dissolved in EtOH (25 mL) and phosphoric acid (85% in water, 486 μL, 7.08 mmol) was added dropwise. The reaction mixture was heated in a sealed tube at 85° C. for 18 h. The precipitate was collected by filtration, washed with cold EtOH (3 mL) and dried to give crude title compound.

### Intermediate 103

### 6-[(3-fluorophenyl)methyl]-7-hydroxy-N-methoxy-N-methylpyrazolo[1,5-a]pyrimidine-5-carboxamide

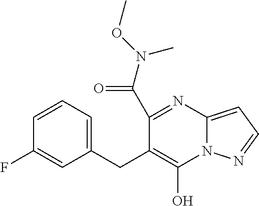 [SMILES: CON(C)C(=O)c1nc2ccnn2c(O)c1Cc1cccc(F)c1]

Intermediate 97 (500 mg, 1.74 mmol) was dissolved in DMF (5 mL) and DIPEA (0.91 mL, 5.22 mmol) and HBTU (990 mg, 2.61 mmol) were added. The reaction mixture was stirred for 30 min and N,O-dimethylhydroxylamine hydrochloride (340 mg, 3.48 mmol) was added. The reaction mixture was stirred at room temperature for 5 h. The solvent was removed in vacuo and the residue was dissolved in EtOAc (75 mL) and washed with sat aq NH₄Cl (100 mL), brine (100 mL), dried (MgSO₄) and concentrated in vacuo. The residue was purified by HPLC to give the title compound (116 mg, 20%) as a white solid.

### Intermediate 104

### 6-[(3,4-Dichlorophenyl)methyl]-7-hydroxy-N-methoxy-N-methylpyrazolo[1,5-a]pyrimidine-5-carboxamide

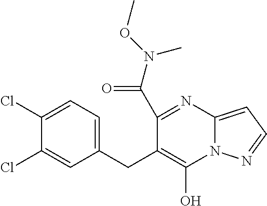 [SMILES: CON(C)C(=O)c1nc2ccnn2c(O)c1Cc1ccc(Cl)c(Cl)c1]

Intermediate 98 (288 mg, 0.85 mmol), EDC hydrochloride (359 mg, 1.87 mmol), HONB (382 mg, 2.13 mmol) and N-ethylmorpholine (271 μL, 2.13 mmol) were dissolved in DMF (10 mL) and stirred for 30 min. N,O-Dimethylhydroxylamine hydrochloride (87.0 mg, 0.89 mmol) was added and the reaction mixture was stirred for 5.5 h. N,O-dimethylhydroxylamine hydrochloride (87.0 mg, 0.89 mmol) was added and the reaction mixture was stirred for 16 h. The reaction mixture was concentrated in vacuo, purified by column chromatography and recrystallised from MeOH to give the title compound (132 mg, 41%) as a white solid.

### Intermediate 105

### 1,5-Diethyl 2-[(4-chlorophenyl)methyl]-3-oxopentanedioate

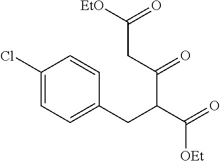 [SMILES: CCOC(=O)CC(=O)C(Cc1ccc(Cl)cc1)C(=O)OCC]

Diethyl 1,3-acetonedicarboxylate (10.0 g, 49.5 mmol) and 4-chlorobenzyl bromide were reacted according to General Procedure C to give the title compound (13.3 g, 82%) as a pale yellow liquid which was used without further purification or characterization.

### Intermediate 106

### Ethyl 2-{6-[(4-chlorophenyl)methyl]-7-hydroxypyrazolo[1,5-a]pyrimidin-5-yl}acetate

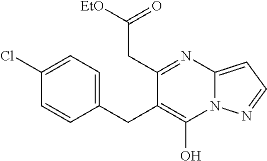 [SMILES: CCOC(=O)Cc1nc2ccnn2c(O)c1Cc1ccc(Cl)cc1]

Intermediate 105 (2.50 g, 7.65 mmol) and 3-aminopyrazole (0.70 g, 8.42 mmol) were suspended in EtOH (30 mL) and phosphoric acid (85% in water, 0.89 mL, 15.3 mmol) was added. The reaction mixture was heated at reflux for 16 h. Water (30 mL) was added and the reaction mixture was stirred for 30 min and cooled to −22° C. The precipitate was collected by filtration, washed with EtOH (50 mL) and dried to give crude title compound (404 mg, 15%) as a white solid which was used without further purification or characterization.

### Intermediate 107

### General Procedure I

### Ethyl 6-[(4-chlorophenyl)methyl]-7-hydroxy-5-methylpyrazolo[1,5-a]pyrimidine-3-carboxylate

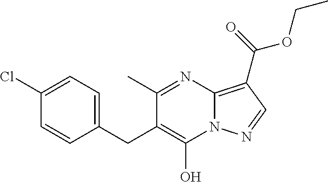 [SMILES: CCOC(=O)c1cnn2c(O)c(Cc3ccc(Cl)cc3)c(C)nc12]

Intermediate 1 (670 mg, 2.60 mmol) and 3-amino-4-carbethoxypyrazole (450 mg, 2.90 mmol) were suspended in EtOH (30 mL) and phosphoric acid (85% in water, 300 μL, 4.37 mmol) was added. The reaction mixture was heated at reflux for 2 d. Water (50 mL) was added and the reaction mixture was stirred for 1 h and cooled to 0° C. for 16 h. The precipitate was collected by filtration, washed with water and dried to give the title compound (670 mg, 73%) as a white solid.

### Intermediates 108-114

Intermediates 108-114 were prepared similarly to General Procedure I; see Table 5 below.

### Intermediate 115

### General Procedure J

### 6-[(4-chlorophenyl)methyl]-7-hydroxy-5-methylpyrazolo[1,5-a]pyrimidine-3-carboxylic acid

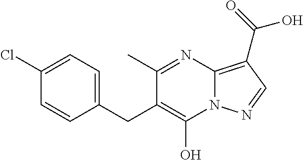 [SMILES: Cc1nc2c(C(=O)O)cnn2c(O)c1Cc1ccc(Cl)cc1]

Intermediate 107 (670 mg, 1.94 mmol) and KOH (1.00 g, 17.8 mmol) were dissolved in water (50 mL) and EtOH (50 mL) and heated at reflux for 2 d. The reaction mixture was acidified to pH 3 with aq phosphoric acid and heated at reflux for 1 h. The precipitate was collected by filtration, washed with water and dried to give the title compound (550 mg, 89%) as a white solid.

### Intermediates 116-122

Intermediates 116-122 were prepared similarly to General Procedure J; see Table 6 below.

TABLE 5
R⁷ ester intermediates

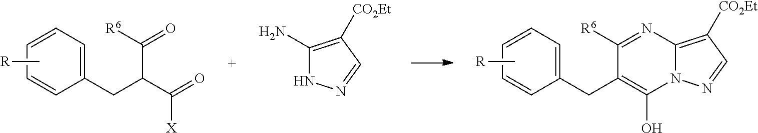

| Int | R | R⁶ | Yield | SM (Int) | Intermediate Name |
|---|---|---|---|---|---|
| 108 | 4-Me | Me | 83% | 5 | Ethyl 7-hydroxy-5-methyl-6-[(4-methylphenyl)methyl]pyrazolo[1,5-a]- |
| | | | | | pyrimidine-3-carboxylate |
| 109 | H | Me | 72% | * | Ethyl 6-benzyl-7-hydroxy-5-methylpyrazolo[1,5-a]pyrimidine-3-carboxylate |
| 110 | 4-CF₃ | Me | 78% | 7 | Ethyl 7-hydroxy-5-methyl-6-{[4-(trifluoromethyl)phenyl]methyl}- |
| | | | | | pyrazolo[1,5-a]pyrimidine-3-carboxylate hydrochloride |
| 111 | 3,4-Cl₂ | Me | 74% | 2 | Ethyl 6-[(3,4-dichlorophenyl)methyl]-7-hydroxy-5-methylpyrazolo[1,5-a]- |
| | | | | | pyrimidine-3-carboxylate |
| 112 | 3-Cl | Me | 49% | 6 | Ethyl 6-[(3-chlorophenyl)methyl]-7-hydroxy-5-methylpyrazolo[1,5-a]- |
| | | | | | pyrimidine-3-carboxylate |
| 113 | 4-Cl | Et | 60% | 11 | Ethyl 6-[(4-chlorophenyl)methyl]-5-ethyl-7-hydroxypyrazolo[1,5-a]- |
| | | | | | pyrimidine-3-carboxylate |
| 114 | 4-Cl | CH₂OMe | 47% | 52 | Ethyl 6-[(4-chlorophenyl)methyl]-7-hydroxy-5-(methoxymethyl)pyrazolo- |
| | | | | | [1,5-a]pyrimidine-3-carboxylate |

*Intermediate beta-keto ester commercially available.

TABLE 6
R⁷ carboxylic acid intermediates

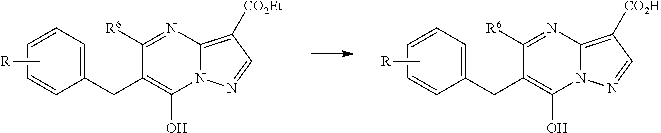

| Int | R | R⁶ | Yield | SM (Int) | Intermediate Name |
|---|---|---|---|---|---|
| 116 | 4-Me | Me | 96% | 108 | 7-Hydroxy-5-methyl-6-[(4-methylphenyl)methyl]pyrazolo[1,5-a]pyrimidine- |
| | | | | | 3-carboxylic acid |
| 117 | H | Me | 95% | 109 | 6-Benzyl-7-hydroxy-5-methylpyrazolo[1,5-a]pyrimidine-3-carboxylic acid |
| 118 | 4-CF₃ | Me | 82% | 110 | 7-Hydroxy-5-methyl-6-{[4-(trifluoromethyl)phenyl]methyl}pyrazolo[1,5- |
| | | | | | a]pyrimidine-3-carboxylic acid |
| 119 | 3,4-Cl₂ | Me | 78% | 111 | 6-[(3,4-Dichlorophenyl)methyl]-7-hydroxy-5-methylpyrazolo[1,5-a]- |
| | | | | | pyrimidine-3-carboxylic acid |
| 120 | 3-Cl | Me | 58% | 112 | 6-[(3-Chlorophenyl)methyl]-7-hydroxy-5-methylpyrazolo[1,5-a]pyrimidine- |
| | | | | | 3-carboxylic acid |
| 121 | 4-Cl | Et | 56% | 113 | 6-[(4-Chlorophenyl)methyl]-5-ethyl-7-hydroxypyrazolo[1,5-a]pyrimidine-3- |
| | | | | | carboxylic acid |
| 122 | 4-Cl | CH₂OMe | 72% | 114 | 6-[(4-Chlorophenyl)methyl]-7-hydroxy-5-(methoxymethyl)pyrazolo[1,5-a]- |
| | | | | | pyrimidine-3-carboxylic acid |

### Example 1

### 6-[(4-Chlorophenyl)methyl]-5-methylpyrazolo[1,5-a]pyrimidin-7-ol

### General Procedure K

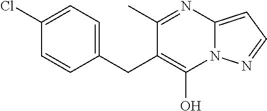 [SMILES: Cc1nc2ccnn2c(O)c1Cc1ccc(Cl)cc1]

Intermediate 1 (5.00 g, 19.6 mmol) and 3-aminopyrazole (1.79 g, 21.6 mmol) were suspended in EtOH (100 mL) and phosphoric acid (85% in water, 2.29 mL, 39.3 mmol) was added. The reaction mixture was heated at reflux for 48 h. Water (30 mL) was added and the reaction mixture was cooled to 4° C. The precipitate was collected by filtration, washed with water and EtOH and dried to give the title compound (4.12 g, 77%) as a white solid. HRMS (ESI⁺) calculated for C₁₄H₁₂ClN₃O: 273.06689. found 273.06696. HPLC: Rf 5.27 min, 100%.

### Example 2

### General Procedure L

### 5-Methyl-6-{[4-(trifluoromethyl)phenyl]methyl}pyrazolo[1,5-a]pyrimidin-7-ol

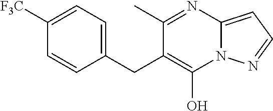 [SMILES: Cc1nc2ccnn2c(O)c1Cc1ccc(C(F)(F)F)cc1]

Intermediate 7 (6.46 mmol) and 3-aminopyrazole (644 mg, 7.75 mmol) were suspended in EtOH (15 mL) and AcOH (2.5 mL) was added. The reaction mixture was heated using a Biotage microwave at 120° C., for 1 h. The precipitate was collected by filtration, washed with EtOH and dried to give the title compound (387 mg, 20%) as a white solid. HRMS (ESI⁺) calculated for C₁₅H₁₂F₃N₃O: 307.093247. found 307.093377. HPLC: Rf 5.40 min, 100%.

### Example 3

### General Procedure M

### 3-[(3-Fluorophenyl)methyl]-2-(pyrazin-2-yl)imidazo[1,5-a]pyrimidin-4-ol

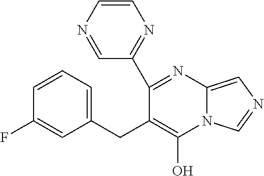 [SMILES: Oc1c(Cc2cccc(F)c2)c(-c2cnccn2)nc2cncn12]

Intermediate 59 (205 mg, 0.71 mmol) and 3-aminopyrazole (71.0 mg, 0.85 mmol) were dissolved in AcOH (5 mL) and heated to 80° C. for 3 d. The reaction mixture was concentrated in vacuo and purified by column chromatography and recrystallisation from EtOH to give the title compound (54.0 mg, 25%) as a pale yellow solid. HRMS (ESI⁺) calculated for C₁₇H₁₂FN₅O: 321.102588. found 321.103018. HPLC: Rf 4.44 min, 100%.

### Example 4

### General Procedure N

### 5-Ethyl-6-{[3-fluoro-5-(trifluoromethyl)phenyl]methyl}pyrazolo[1,5-a]pyrimidin-7-ol

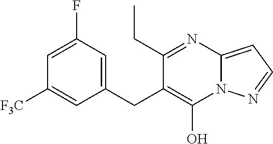 [SMILES: CCc1nc2ccnn2c(O)c1Cc1cc(F)cc(C(F)(F)F)c1]

Intermediate 15 and 3-aminopyrazole (622 mg, 7.49 mmol) were suspended in EtOH (10 mL) and phosphoric acid (85% in water, 1.00 mL, 14.6 mmol) was added. The reaction mixture was heated using a Biotage microwave (170° C., absorption high) for 1 h. The precipitate was collected by filtration, washed from EtOH (2×10 mL) and dried to give the title compound (1.15 g, 54%) as a white solid. HRMS (ESI⁺) calculated for C₁₆H₁₃F₄N₃O: 339.099475. found 339.100165. HPLC: Rf 5.86 min, 98.2%.

### Example 5

### 6-(3-Fluorophenoxy)-5-methylpyrazolo[1,5-a]pyrimidin-7-ol

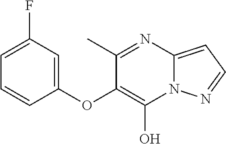 [SMILES: Cc1nc2ccnn2c(O)c1Oc1cccc(F)c1]

Intermediate 60 (190 mg, 0.84 mmol) and 3-aminopyrazole (77.0 mg, 0.92 mmol) were dissolved in EtOH (5 mL) and the reaction mixture was heated at reflux for 1 h. The precipitate was collected by filtration and dried to give the title compound (80.0 mg, 38%) as a white solid. HRMS (ESI⁺) calculated for C₁₃H₁₀FN₃O₂: 259.075705. found 259.076515. HPLC: Rf 4.55 min, 99.8%.

### Example 6

### General Procedure O

### Methyl 6-[(4-chlorophenyl)methyl]-7-hydroxypyrazolo[1,5-a]pyrimidine-5-carboxylate

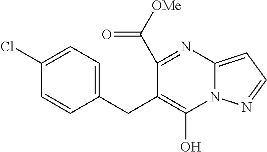 [SMILES: COC(=O)c1nc2ccnn2c(O)c1Cc1ccc(Cl)cc1]

Intermediate 78 (300 mg, 1.05 mmol) and 3-aminopyrazole (87.6 mg, 1.05 mmol) were dissolved in AcOH (2 mL) and the reaction mixture was heated at reflux for 15 h. Et₂O (10 mL) was added and the resulting precipitate was collected by filtration and washed with Et₂O (5×10 mL). The residue was purified by column chromatography to give the title compound (45.2 mg, 14%) as an orange solid. HRMS (ESI⁺) calculated for C₁₅H₁₂ClN₃O₃: 317.056719. found 317.053609. HPLC: Rf 5.68 min, 96.9%.

### Examples 7-77

Examples 7-77 were prepared similarly to General Procedures K-O, by reacting Intermediate beta-keto esters (Table 1) with 3-aminopyrazoles; see Table 7 below.

### Example 78

### 6-[(3-Fluorophenyl)amino]-5-methylpyrazolo[1,5-a]pyrimidin-7-ol

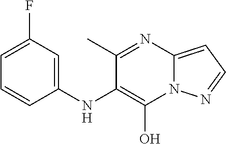 [SMILES: Cc1nc2ccnn2c(O)c1Nc1cccc(F)c1]

Intermediate 93 (473 mg, 1.59 mmol) was suspended in EtOH (7.5 mL), TiCl₃ (1.23 mL, 30 wt % in 2 M aq HCl, 2.39 mmol) was added and the reaction mixture was stirred for 2 h. A solution of 3-aminopyrazole (132 mg, 1.59 mmol) in EtOH (2.5 mL) was added and the reaction mixture was heated at reflux for 75 min and stirred at room temperature for 16 h. The reaction mixture was basified to pH 8 with Et₃N and the solvents were removed in vacuo. The residue was purified by column chromatography and triturated from Et₂O. The residue was suspended in water (5 mL) and stirred for 1 h. The precipitate was collected by filtration and dried to give the title compound (56.0 mg, 14%) as a white solid. HRMS (ESI⁺) calculated for C₁₃H₁₁FN₄O: 258.091689. found 258.092659. HPLC: Rf 4.54 min, 99.6% purity.

TABLE 7
Preparation of pyrazolo[1,5-a]pyrimidines

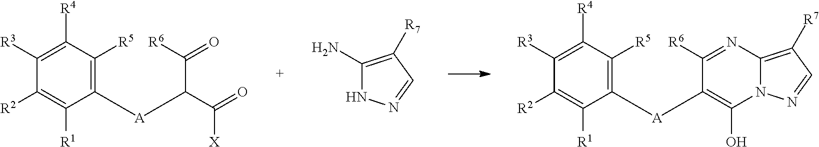

| Ex | Structure | Name | Int | Proc | Yield | HRMS (ESI⁺)/HPLC data |
|---|---|---|---|---|---|---|
| 7 | 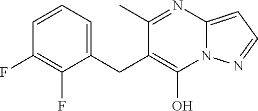 [SMILES: Cc1nc2ccnn2c(O)c1Cc1cccc(F)c1F] | 6-[(2,3-Difluorophenyl)methyl]-5-methyl- pyrazolo[1,5-a]pyrimidine-7-ol | 3 | K | 26% | Calculated for C₁₄H₁₁F₂N₃O: 275.087018. found 275.087618. HPLC: Rf 4.95 min, 100%. |
| 8 | 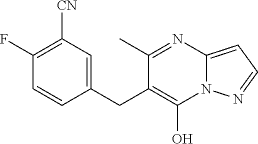 [SMILES: Cc1nc2ccnn2c(O)c1Cc1ccc(F)c(C#N)c1] | 2-Fluoro-5-({7-hydroxy-5-methylpyrazolo- (1,5-a]pyrimidin-6-yl}methyl)benzonitrile | 8 | K | 43% | Calculated for C₁₅H₁₁FN₄O: 282.091689, found 282.092099. HPLC: Rf 4.75 min, 100%. |
| 9 | 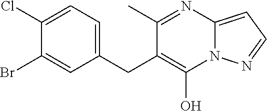 [SMILES: Cc1nc2ccnn2c(O)c1Cc1ccc(Cl)c(Br)c1] | 6-[(3-Bromo-4-chlorophenyl)methyl]-5- methylpyrazolo[1,5-a]pyrimidin-7-ol | 9 | K | 74% | Calculated for C₁₄H₁₁BrClN₃O: 350.977402, found 350.977892. HPLC: Rf 3.54 min (gradient 50-100%), 100%. |
| 10 | 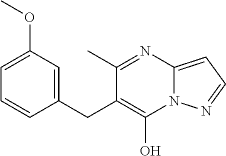 [SMILES: COc1cccc(Cc2c(C)nc3ccnn3c2O)c1] | 6-[(3-Methoxyphenyl)methyl]-5-methyl- pyrazolo[1,5-a]pyrimidin-7-ol | 10 | K | 9% | Calculated for C₁₅H₁₅N₃O₂: 269.116427, found 269.116117. HPLC: Rf 4.75 min, 97.8%. |
| 11 | 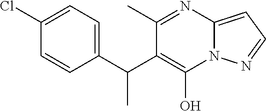 [SMILES: Cc1nc2ccnn2c(O)c1C(C)c1ccc(Cl)cc1] | 6-[1-(4-Chlorophenyl)ethyl]-5-methyl- pyrazolo[1,5-a]pyrimidin-7-ol | 4 | K | 62% | Calculated for C₁₅H₁₄ClN₃O: 287.09254, found 287.08269. HPLC: Rf 5.58 min, 100%. |
| 12 | 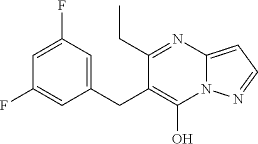 [SMILES: CCc1nc2ccnn2c(O)c1Cc1cc(F)cc(F)c1] | 6-[(3,5-difluorophenyl)methyl]-5-ethyl- pyrazolo[1,5-a]pyrimidin-7-ol | 12 | K | 34% | Calculated for C₁₅H₁₃F₂N₃O: 289.102668, found 289.103328. HPLC: Rf 5.23 min, 99.5%. |
| 13 | 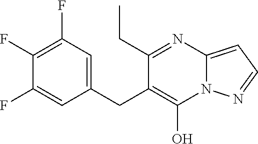 [SMILES: CCc1nc2ccnn2c(O)c1Cc1cc(F)c(F)c(F)c1] | 5-Ethyl-6-[(3,4,5-trifluorophenyl)- methyl]pyrazolo[1,5-a]pyrimidin-7-ol | 13 | N | 29% | Calculated for C₁₅H₁₂F₃N₃O: 307.093247, found 307.095047. HPLC: Rf 5.55 min, 97.6%. |
| 14 | 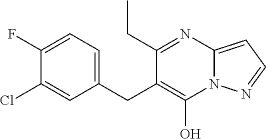 [SMILES: CCc1nc2ccnn2c(O)c1Cc1ccc(F)c(Cl)c1] | 6-[(3-Chloro-4-fluorophenyl)methyl]-5- ethylpyrazolo[1,5-a]pyrimidin-7-ol | 14 | N | 42% | Calculated for C₁₅H₁₃ClFN₃O: 305.073118, found 305.072538. HPLC: Rf 5.52 min, 97.9%. |
| 15 | 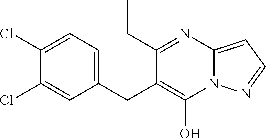 [SMILES: CCc1nc2ccnn2c(O)c1Cc1ccc(Cl)c(Cl)c1] | 6-[(3,4-Dichlorophenyl)methyl]-5- ethylpyrazolo[1,5-a]pyrimidin-7-ol | 16 | K | 3% | Calculated for C₁₅H₁₃Cl₂N₃O: 321.043567, found 321.044267. HPLC: Rf 5.82 min, 98.4%. |
| 16 | 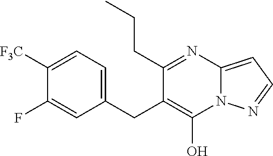 [SMILES: CCCc1nc2ccnn2c(O)c1Cc1ccc(C(F)(F)F)c(F)c1] | 6-{[3-Fluoro-4-(trifluoromethyl)phenyl]- methyl}-5-propylpyrazolo[1,5-a]- pyrimidin-7-ol | 17 | N | 24% | Calculated for C₁₇H₁₅F₄N₃O: 353.115125, found 353.115645. HPLC: Rf 6.12 min, 100%. |
| 17 | 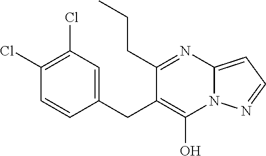 [SMILES: CCCc1nc2ccnn2c(O)c1Cc1ccc(Cl)c(Cl)c1] | 6-[(3,4-Dichlorophenyl)methyl]-5-propyl- pyrazolo[1,5-a]pyrimidin-7-ol | 18 | K | 2% | Calculated for C₁₆H₁₅Cl₂N₃O: 335.059218, found 335.059148. HPLC: Rf 6.16 min, 98.7%. |
| 18 | 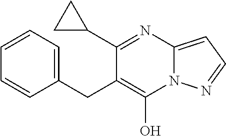 [SMILES: Oc1c(Cc2ccccc2)c(C2CC2)nc2ccnn12] | 6-Benzyl-5-cyclopropylpyrazolo[1,5-a]- pyrimidin-7-ol | 19 | K | 22% | Calculated for C₁₆H1₅N₃O: 265.121512, found 265.121832. HPLC: Rf 5.26 min, 100%. |
| 19 | 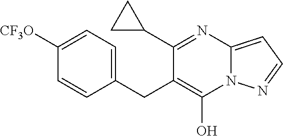 | 5-Cyclopropyl-6-{[4-(trifluoromethoxy)- phenyl]methyl}pyrazolo[1,5-a]pyrimidin- 7-ol | 29 | K | 20% | Calculated for C₁₇H₁₄F₃N₃O₂: 349.103811, found 349.104461. HPLC: Rf 6.05 min, 99.6%. |
| 20 | 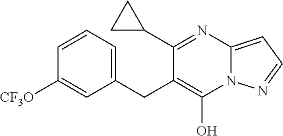 | 5-Cyclopropyl-6-{[3-(trifluoromethoxy)- phenyl]methyl}pyrazolo[1,5-a]pyrimidin- 7-ol | 21 | K | 22% | Calculated for C₁₇H₁₄F₃N₃O₂: 349.103811, found 349.104621. HPLC: Rf 6.01 min, 99.3%. |
| 21 | 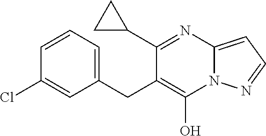 [SMILES: Oc1c(Cc2cccc(Cl)c2)c(C2CC2)nc2ccnn12] | 6-[(3-Chlorophenyl)methyl]-5-cyclo- propylpyrazolo[1,5-a]pyrimidin-7-ol | 22 | N | 23% | Calculated for C₁₆H₁₄ClN₃O: 299.08254, found 299.08395. HPLC: Rf 5.70 min, 98.9%. |
| 22 | 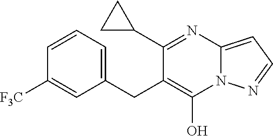 [SMILES: Oc1c(Cc2cccc(C(F)(F)F)c2)c(C2CC2)nc2ccnn12] | 5-Cycloproyl-6-{[3-(trifluoromethyl)- phenyl]methyl}pyrazolo[1,5-a]pyrimidin- 7-ol | 23 | K | 11% | Calculated for C1₇H₁₄F₃N₃O: 333.108897, found 331.110277. HPLC: Rf 5.87 min, 99.4%. |
| 23 | 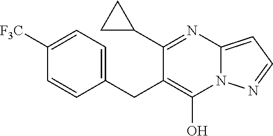 [SMILES: Oc1c(Cc2ccc(C(F)(F)F)cc2)c(C2CC2)nc2ccnn12] | 5-Cyclopropyl-6-[4-(trifluoromethyl)- phenyl]methyl}pyrazolo[1,5-a]pyrimidin- 7-ol | 24 | K | 43% | Calculated for C1₇H₁₄F₃N₃O: 333.108897, found 333.109427. HPLC: Rf 5.92 min, 99.1%. |
| 24 | 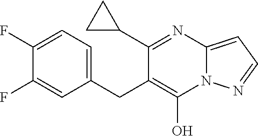 [SMILES: Oc1c(Cc2ccc(F)c(F)c2)c(C2CC2)nc2ccnn12] | 5-Cyclopropyl-6-[(3,4-difluorophenyl)- methyl]pyrazolo[1,5-a]pyrimidin-7-ol | 25 | N | 10% | Calculated for C₁₆H₁₃F₂N₃O: 301.102668, found 301.104118. HPLC: Rf 5.51 min, 99.5%. |
| 25 | 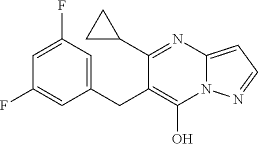 [SMILES: Oc1c(Cc2cc(F)cc(F)c2)c(C2CC2)nc2ccnn12] | 5-Cyclopropyl-6-[(3,5-difluorophenyl)- methyl]pyrazolo[1,5-a]pyrimidin-7-ol | 26 | N | 10% | Calculated for C₁₆H₁₃F₂N₃O: 301.102668, found 301.102968. HPLC: Rf 5.50 min, 97%. |
| 26 | 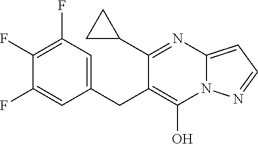 [SMILES: Oc1c(Cc2cc(F)c(F)c(F)c2)c(C2CC2)nc2ccnn12] | 5-Cyclopropyl-6-[(3,4,5-trifluorophenyl)- methyl]pyrazolo[1,5-a]pyrimidin-7-ol | 27 | N | 17% | Calculated for C₁₆H₁₂F₃N₃O: 319.093247, found 319.095197. HPLC: Rf 5.70 min, 99.2%. |
| 27 | 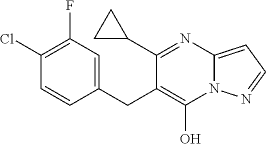 [SMILES: Oc1c(Cc2ccc(Cl)c(F)c2)c(C2CC2)nc2ccnn12] | 6-[(4-Chloro-3-fluorophenyl)methyl]-5- cyclopropylpyrazolo[1,5-a]pyrimidin-7-ol | 28 | N | 4% | Calculated for C₁₆H₁₃ClFN₃O: 317.073118, found 317.073738. HPLC: Rf 5.81 min, 97.5%. |
| 28 | 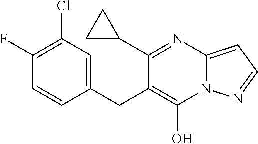 [SMILES: Oc1c(Cc2ccc(F)c(Cl)c2)c(C2CC2)nc2ccnn12] | 6-[(3-Chloro-4-fluorophenyl)methyl]-5- cyclopropylpyrazolo[1,5-a]pyrimidin-7-ol | 29 | N | 2% | Calculated for C₁₆H₁₃ClFN₃O: 317.073118, found 317.073978. HPLC: Rf 5.78 min, 98.5%. |
| 29 | 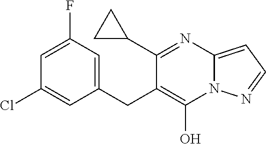 [SMILES: Oc1c(Cc2cc(F)cc(Cl)c2)c(C2CC2)nc2ccnn12] | 6-[(3-Chloro-5-fluorophenyl)methyl]-5- cyclopropylpyrazolo[1,5-a]pyrimidin-7-ol | 30 | N | 7% | Calculated for C₁₆H₁₃ClFN₃O: 317.073118, found 317.073828. HPLC: Rf 5.85 min, 100%. |
| 30 | 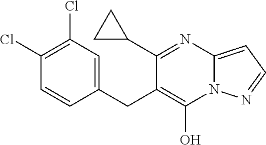 [SMILES: Oc1c(Cc2ccc(Cl)c(Cl)c2)c(C2CC2)nc2ccnn12] | 5-Cyclopropyl-6-[(3,4-dichlorophenyl)- methyl]pyrazolo[1,5-a]pyrimidin-7-ol | 31 | N | 2% | Calculated for C₁₆H₁₃Cl₂N₃O: 333.043567, found 333.043537. HPLC: Rf 5.97 min, 96.7%. |
| 31 | 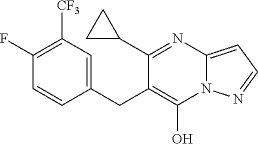 [SMILES: Oc1c(Cc2ccc(F)c(C(F)(F)F)c2)c(C2CC2)nc2ccnn12] | 5-Cyclopropyl-6-{[4-fluoro-3-(trifluoro- methyl)phenyl]methyl}pyrazolo[1,5-a]- pyrimidin-7-ol | 32 | N | 1% | Calculated for C₁₇H₁₃F₄N₃O: 351.099475, found 351.099975. HPLC: Rf 5.95 min, 98.2%. |
| 32 | 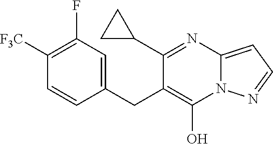 [SMILES: Oc1c(Cc2ccc(C(F)(F)F)c(F)c2)c(C2CC2)nc2ccnn12] | 5-Cyclopropyl-6-{[3-fluoro-4- (trifluoromethyl)phenyl]methyl}pyrazolo[1, 5-a]pyrimidin-7-ol | 33 | N | 4% | Calculated for C₁₇H₁₃F₄N₃O: 351.099475, found 351.100545. HPLC: Rf 5.98 min, 100%. |
| 33 | 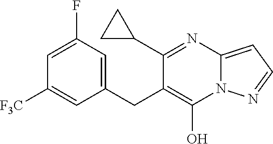 [SMILES: Oc1c(Cc2cc(F)cc(C(F)(F)F)c2)c(C2CC2)nc2ccnn12] | 5-Cyclopropyl-6-{[3-fluoro-5-(trifluoro- methyl)phenyl]methyl}pyrazolo[1,5-a]- pyrimidin-7-ol | 34 | N | 7% | Calculated for C₁₇H₁₃F₄N₃O: 351.099475, found 351.100015. HPLC: Rf 6.00 min, 100%. |
| 34 | 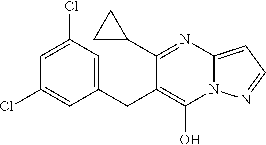 [SMILES: Oc1c(Cc2cc(Cl)cc(Cl)c2)c(C2CC2)nc2ccnn12] | 5-Cyclopropyl-6-[(3,5-dichlorophenyl)- methyl]pyrazolo[1,5-a]pyrimidin-7-ol | 35 | K | 22% | Calculated for C₁₆H₁₃Cl₂N₃O: 333.043567, found 333.044467. HPLC: Rf 6.20 min, 99.4%. |
| 35 | 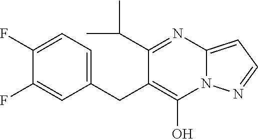 [SMILES: CC(C)c1nc2ccnn2c(O)c1Cc1ccc(F)c(F)c1] | 6-[(3,4-Difluorophenyl)methyl]-5-(propan- 2-yl)pyrazolo[1,5-a]pyrimidin-7-ol | 36 | N | 3% | Calculated for C₁₆H₁₅F₂N₃O: 303.118319, found 303.121319. HPLC: Rf 5.70 min, 98.6%. |
| 36 | 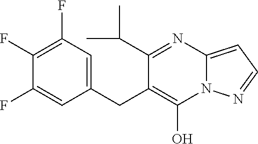 [SMILES: CC(C)c1nc2ccnn2c(O)c1Cc1cc(F)c(F)c(F)c1] | 5-(Propan-2-yl)-6-[(3,4,5-trifluorophenyl)- methyl]pyrazolo[1,5-a]pyrimidin-7-ol | 37 | N | 7% | Calculated for C₁₆H₁₄F₃N₃O: 321.108897, found 321.109327. HPLC: Rf 5.86 min, 99.8%. |
| 37 | 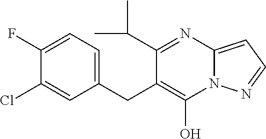 [SMILES: CC(C)c1nc2ccnn2c(O)c1Cc1ccc(F)c(Cl)c1] | 6-[(3-Chloro-4-fluorophenyl)methyl]-5- (propan-2-yl)pyrazolo[1,5-a]pyrimidin-7- ol | 38 | N | 12% | Calculated for C₁₆H₁₅ClFN₃O: 319.088768, found 319.089448. HPLC: Rf 5.81 min, 99.5%. |
| 38 | 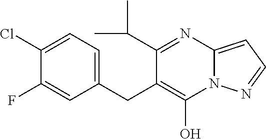 [SMILES: CC(C)c1nc2ccnn2c(O)c1Cc1ccc(Cl)c(F)c1] | 6-[(4-Chloro-3-fluorophenyl)methyl]-5- (propan-2-yl)pyrazolo[1,5-a]pyrimidin-7- ol | 39 | L | 9% | Calculated for C₁₆H₁₅ClFN₃O: 319.088768, found 319.088528. HPLC: Rf 5.85 min, 98.8%. |
| 39 | 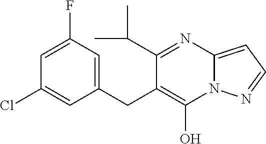 [SMILES: CC(C)c1nc2ccnn2c(O)c1Cc1cc(F)cc(Cl)c1] | 6-[(3-Chloro-5-fluorophenyl)methyl]-5- (propan-2-yl)pyrazolo[1,5-a]pyrimidin-7- ol | 40 | K | 10% | Calculated for C₁₆H₁₅ClFN₃O: 319.088768, found 319.091308. HPLC: Rf 6.02 min, 98%. |
| 40 | 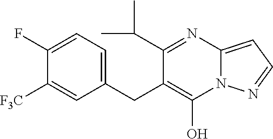 [SMILES: CC(C)c1nc2ccnn2c(O)c1Cc1ccc(F)c(C(F)(F)F)c1] | 6-{[4-Fluoro-3-(trifluoromethyl)phenyl]- methyl}-5-(propan-2-yl)pyrazolo[1,5-a]- pyrimidin-7-ol | 41 | N | 4% | Calculated for C₁₇H₁₅F₄N₃O: 353.115125, found 353.115965. HPLC: Rf 6.11 min, 99.4%. |
| 41 | 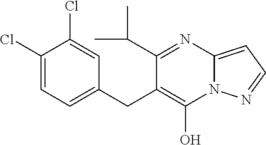 [SMILES: CC(C)c1nc2ccnn2c(O)c1Cc1ccc(Cl)c(Cl)c1] | 6-[(3,4-Dichlorophenyl)methyl]-5-(propan- 2-yl)pyrazolo[1,5-a]pyrimidin-7-ol | 42 | K | 6% | Calculated for C₁₆H₁₅Cl₂N₃O: 335.059217, found 335.060297. HPLC: Rf 6.23 min, 99.9%. |
| 42 | 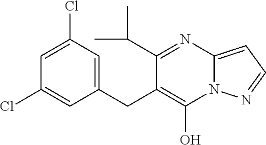 [SMILES: CC(C)c1nc2ccnn2c(O)c1Cc1cc(Cl)cc(Cl)c1] | 6-[(3,5-Dichlorophenyl)methyl]-5-(propan- 2-yl)pyrazolo[1,5-a]pyrimidin-7-ol | 43 | N | 15% | Calculated for C₁₆H₁₅Cl₂N₃O: 335.059218, found 335.060738. HPLC: Rf 6.39 min, 100%. |
| 43 | 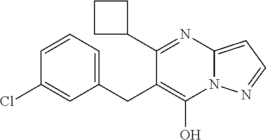 [SMILES: Oc1c(Cc2cccc(Cl)c2)c(C2CCC2)nc2ccnn12] | 6-[(3-Chlorophenyl)methyl]-5-cyclobutyl- pyrazolo[1,5-a]pyrimidin-7-ol | 44 | N | 14% | Calculated for C₁₇H₁₆ClN₃O: 313.09819, found 313.09785. HPLC: Rf 6.11 min, 99.4%. |
| 44 | 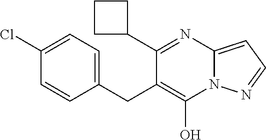 [SMILES: Oc1c(Cc2ccc(Cl)cc2)c(C2CCC2)nc2ccnn12] | 6-[(4-Chlorophenyl)methyl]-5-cyclobutyl- pyrazolo[1,5-a]pyrimidin-7-ol | 45 | K | 26% | Calculated for C₁₇H₁₆ClN₃O: 313.09819, found 313.09849. HPLC: Rf 6.16 min, 99.5%. |
| 45 | 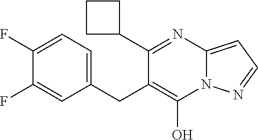 [SMILES: Oc1c(Cc2ccc(F)c(F)c2)c(C2CCC2)nc2ccnn12] | 5-Cyclobutyl-6-[(3,4-difluorophenyl)- methyl]pyrazolo[1,5-a]pyrimidin-7-ol | 46 | N | 35% | Calculated for C₁₇H₁₅F₂N₃O: 315.118319, found 315.119129. HPLC: Rf 5.89 min, 99.6%. |
| 46 | 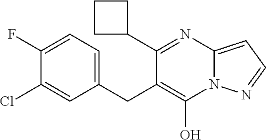 [SMILES: Oc1c(Cc2ccc(F)c(Cl)c2)c(C2CCC2)nc2ccnn12] | 6-[(3-Chloro-4-fluorophenyl)methyl]-5- cyclobutylpyrazolo[1,5-a]pyrimidin-7-ol | 47 | N | 5% | Calculated for C₁₇H₁₅ClFN₃O: 331.088768, found 331.089958. HPLC: Rf 6.16 min, 97.6%. |
| 47 | 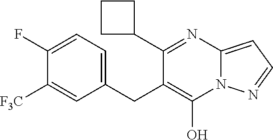 [SMILES: Oc1c(Cc2ccc(F)c(C(F)(F)F)c2)c(C2CCC2)nc2ccnn12] | 5-Cyclobutyl-6-{[4-fluoro-3-(trifluoro- methyl)phenyl]methyl}pyrazolo[1,5-a]- pyrimidin-7-ol | 48 | N | 10% | Calculated for C₁₈H₁₅F₄N₃O: 365.115125, found 365.115105. HPLC: Rf 6.27 min, 100%. |
| 48 | 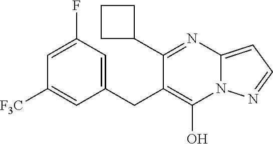 [SMILES: Oc1c(Cc2cc(F)cc(C(F)(F)F)c2)c(C2CCC2)nc2ccnn12] | 5-Cyclobutyl-6-{[3-fluoro-5-(trifluoro- methyl)phenyl]methyl}pyrazolo[1,5-a]- pyrimidin-7-ol | 49 | K | 25% | Calculated for C₁₈H₁₅F₄N₃O: 365.115125, found 365.116195. HPLC: Rf 6.31 min, 100%. |
| 49 | 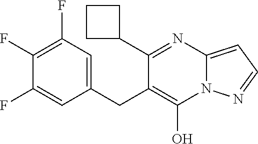 [SMILES: Oc1c(Cc2cc(F)c(F)c(F)c2)c(C2CCC2)nc2ccnn12] | 5-Cyclobutyl-6-[(3,4,5-trifluorophenyl)- methyl]pyrazolo[1,5-a]pyrimidin-7-ol | 50 | N | 10% | Calculated for C₁₇H₁₄F₃N₃O: 333.108897, found 333.109487. HPLC: Rf 6.05 min, 99.5%. |
| 50 | 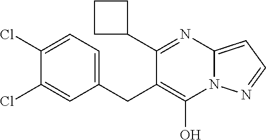 [SMILES: Oc1c(Cc2ccc(Cl)c(Cl)c2)c(C2CCC2)nc2ccnn12] | 5-Cyclobutyl-6-[(3,4-dichlorophenyl)- methyl]pyrazolo[1,5-a]pyrimidin-7-ol | 51 | N | 16% | Calculated for C₁₇H₁₅Cl₂N₃O: 347.059218, found 347.060058. HPLC: Rf 6.51 min, 97.2%. |
| 51 | 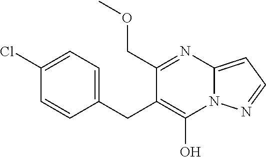 [SMILES: COCc1nc2ccnn2c(O)c1Cc1ccc(Cl)cc1] | 6-[(4-Chlorophenyl)methyl]-5-(methoxy- methyl)pyrazolo[1,5-a]pyrimidin-7-ol | 52 | K | 59% | Calculated for C₁₅H₁₄ClN₃O₂: 303.077454, found 303.078734. HPLC: Rf 5.30 min, 98.4%. |
| 52 | 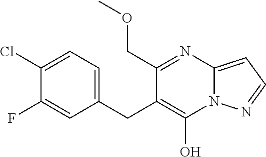 [SMILES: COCc1nc2ccnn2c(O)c1Cc1ccc(Cl)c(F)c1] | 6-[(4-Choro-3-fluorophenyl)methyl]-5- (methoxymethyl)pyrazolo[1,5-a]pyrimidin- 7-ol | 53 | K | 34% | Calculated for C₁₅H₁₃ClFN₃O₂: 321.068033, found 321.069303. HPLC: Rf 5.39 min, 99.7%. |
| 53 | 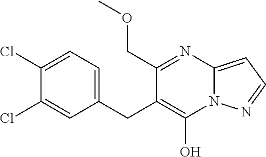 [SMILES: COCc1nc2ccnn2c(O)c1Cc1ccc(Cl)c(Cl)c1] | 6-[(3,4-Dichlorophenyl)methyl]-5- (methoxymethyl)pyrazolo[1,5-a]pyrimidin- 7-ol | 54 | K | 46% | Calculated for C₁₅H₁₃Cl₂N₃O₂: 337.038482, found 337.039602. HPLC: Rf 5.71 min, 98.7%. |
| 54 | 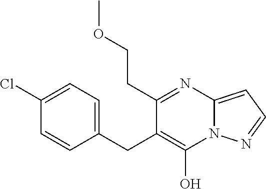 [SMILES: COCCc1nc2ccnn2c(O)c1Cc1ccc(Cl)cc1] | 6-[(4-Chlorophenyl)methyl]-5-(2- methoxyethyl)pyrazolo[1,5-a]pyrimidin-7- ol | 55 | K | 70% | Calculated for C₁₆H₁₆ClN₃O₂: 317.093104, found 317.094414. HPLC: Rf 5.45 min, 97.5%. |
| 55 | 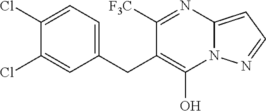 [SMILES: Oc1c(Cc2ccc(Cl)c(Cl)c2)c(C(F)(F)F)nc2ccnn12] | 6-[(3,4-Dichlorophenyl)methyl]-5- (trifluoromethyl)pyrazolo[1,5-a]pyrimidin- 7-ol | 56 | N | 1% | Calculated for C₁₄H₈Cl₂F₃N₃O: 360.999652, found 361.000142. HPLC: Rf 6.64 min, 100%. |
| 56 | 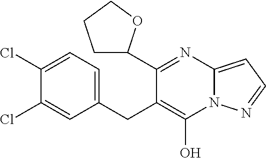 [SMILES: Oc1c(Cc2ccc(Cl)c(Cl)c2)c(C2CCCO2)nc2ccnn12] | 6-[(3,4-Dichlorophenyl)methyl]-5-(oxolan- 2-yl)pyrazolo[1,5-a]pyrimidin-7-ol | 57 | K | 20% | Calculated for C₁₇H₁₅Cl₂N₃O₂: 363.054132, found 363.055672. HPLC: Rf 6.07 min, 99.6%. |
| 57 | 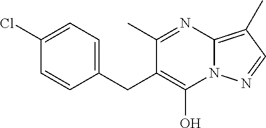 [SMILES: Cc1nc2c(C)cnn2c(O)c1Cc1ccc(Cl)cc1] | 6-[(4-Chlorophenyl)methyl]-3,5-dimethyl- pyrazolo[1,5-a]pyrimidin-7-ol | 1 | K* | 2% | Calculated for C₁₅H₁₄ClN₃O: 287.08254, found 287.08253. HPLC: Rf 5.46 min, 100%. |
| 58 | 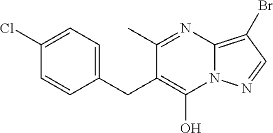 [SMILES: Cc1nc2c(Br)cnn2c(O)c1Cc1ccc(Cl)cc1] | 3-Bromo-6-[(4-chlorophenyl)methyl]-5- methylpyrazolo[1,5-a]pyrimidin-7-ol | 1 | K* | 30% | Calculated for C₁₄H₁₁BrClN₃O: 350.977402, found 350.978562. HPLC: Rf 5.71 min, 99.3%. |
| 59 | 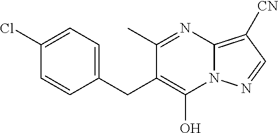 [SMILES: Cc1nc2c(C#N)cnn2c(O)c1Cc1ccc(Cl)cc1] | 6-[(4-Chlorophenyl)methyl]-7-hydroxy-5- methylpyrazolo[1,5-a]pyrimidine-3- carbonitrile | 1 | K* | 81% | Calculated for C₁₅H₁₁ClN₄O: 298.062139, found 298.062949. HPLC: Rf 5.42 min, 100%. |
| 60 | 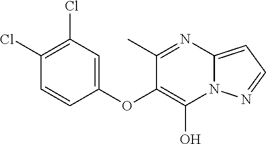 [SMILES: Cc1nc2ccnn2c(O)c1Oc1ccc(Cl)c(Cl)c1] | 6-(3,4-Dichlorophenoxy)-5-methyl- pyrazolo[1,5-a]pyrimidin-7-ol | 61 | M | 41% | Calculated for C₁₃H₉Cl₂N₃O₂: 309.007182, found 309.005812. HPLC: Rf 5.45 min, 97.3%. |
| 61 | 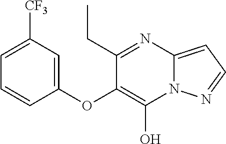 [SMILES: CCc1nc2ccnn2c(O)c1Oc1cccc(C(F)(F)F)c1] | 5-Ethyl-6-[3-(trifluoromethyl)phenoxy] pyrazolo[1,5-a]pyrimidin-7-ol | 62 | M | 1% | Calculated for C₁₅H₁₂F₃N₃O₂: 323.088161, found 323.089041. HPLC: Rf 5.88 min, 100%. |
| 62 | 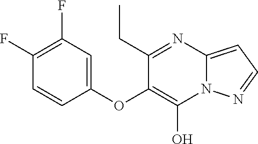 [SMILES: CCc1nc2ccnn2c(O)c1Oc1ccc(F)c(F)c1] | 6-(3,4-Difluorophenoxy)-5-ethyl- pyrazolo[1,5-a]pyrimidin-7-ol | 63 | K | 28% | Calculated for C₁₄H₁₁F₂N₃O₂: 291.081933, found 291.083153. HPLC: Rf 5.23 min, 100%. |
| 63 | 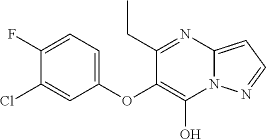 [SMILES: CCc1nc2ccnn2c(O)c1Oc1ccc(F)c(Cl)c1] | 6-(3-Chloro-4-fluorophenoxy)-5-ethyl- pyrazolo[1,5-a]pyrimidin-7-ol | 64 | K | 23% | Calculated for C₁₄H₁₁ClFN₃O₂: 307.052383, found 307.053573. HPLC: Rf 5.50 min, 99.2%. |
| 64 | 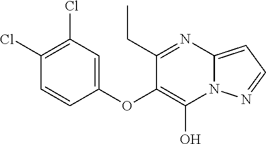 [SMILES: CCc1nc2ccnn2c(O)c1Oc1ccc(Cl)c(Cl)c1] | 6-(3,4-Dichlorophenoxy)-5-ethyl- pyrazolo[1,5-a]pyrimidin-7-ol | 65 | K | 4% | Calculated for C₁₄H₁₁Cl₂N₃O₂: 323.022832, found 323.022852. HPLC: Rf 4.15 min, 98.6%. |
| 65 | 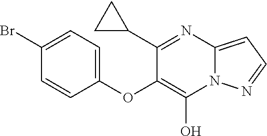 [SMILES: Oc1c(Oc2ccc(Br)cc2)c(C2CC2)nc2ccnn12] | 6-(4-Bromophenoxy)-5-cyclopropyl- pyrazolo[1,5-a]pyrimidin-7-ol | 66 | L | 16% | Calculated for C₁₅H₁₂BrN₃O₂: 345.011289, found 345.011549. HPLC: Rf 5.60 min, 100%. |
| 66 | 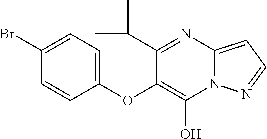 [SMILES: CC(C)c1nc2ccnn2c(O)c1Oc1ccc(Br)cc1] | 6-(4-Bromophenoxy)-5-(propan-2-yl)- pyrazolo[1,5-a]pyrimidin-7-ol | 67 | L | 7% | Calculated for C₁₅H₁₄BrN₃O₂: 347.026939, found 347.027539. HPLC: Rf 5.86 min, 99.2%. |
| 67 | 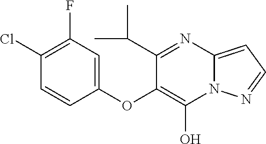 [SMILES: CC(C)c1nc2ccnn2c(O)c1Oc1ccc(Cl)c(F)c1] | 6-(4-Chloro-3-fluorophenoxy)-5-(propan- 2-yl)pyrazolo[1,5-a]pyrimidin-7-ol | 68 | L | 2% | Calculated for C₁₅H₁₃ClFN₃O₂: 321.068033, found 321.069863. HPLC: Rf 5.86 min, 100%. |
| 68 | 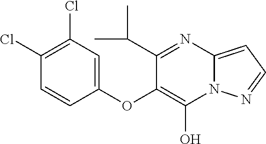 [SMILES: CC(C)c1nc2ccnn2c(O)c1Oc1ccc(Cl)c(Cl)c1] | 6-(3,4-diChlorophenoxy)-5-(propan-2-yl)- pyrazolo[1,5-a]pyrimidin-7-ol | 69 | L | 5% | Calculated for C₁₅H₁₃Cl₂N₃O₂: 337.038482, found 337.039552. HPLC: Rf 6.13 min, 99.3%. |
| 69 | 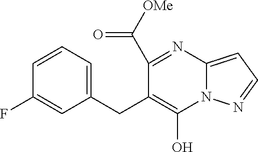 [SMILES: COC(=O)c1nc2ccnn2c(O)c1Cc1cccc(F)c1] | Methyl 6-[(3-fluorophenyl)methyl]-7- hydroxypyrazolo[1,5-a]pyrimidine-5- carboxylate | 80 | O | 59% | Calculated for C₁₅H₁₂FN₃O₃: 301.08627, found 301.08687. HPLC: Rf 5.21 min, 97.3%. |
| 70 | 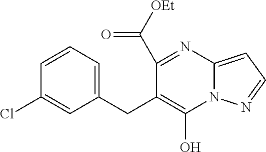 [SMILES: CCOC(=O)c1nc2ccnn2c(O)c1Cc1cccc(Cl)c1] | Ethyl 6-[(3-chlorophenyl)methyl]-7- hydroxypyrazolo[1,5-a]pyrimidine-5- carboxylate | 79 | O | 39% | Calculated for C₁₆H₁₄ClN₃O₃: 331.072369, found 331.071989. HPLC: Rf 5.88 min, 100%. |
| 71 | 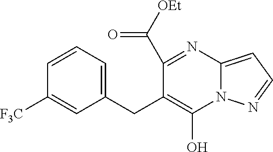 [SMILES: CCOC(=O)c1nc2ccnn2c(O)c1Cc1cccc(C(F)(F)F)c1] | Ethyl 7-hydroxy-6-{[3-(trifluoromethyl)- phenyl]methyl}pyrazolo[1,5-a]pyrimidine- 5-carboxylate | 81 | O | 36% | Calculated for C₁₇H₁₄F₃N₃O₃: 365.098726, found 365.098716. HPLC: Rf 5.96 min, 98.6%. |
| 72 | 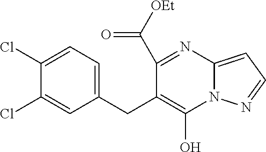 [SMILES: CCOC(=O)c1nc2ccnn2c(O)c1Cc1ccc(Cl)c(Cl)c1] | Ethyl 6-[(3,4-dichlorophenyl)methyl]-7- hydroxypyrazolo[1,5-a]pyrimidine-5- carboxylate | 84 | O | 25% | Calculated for C₁₆H₁₃Cl₂N₃O₃: 365.033397, found 365.034897. HPLC: Rf 6.32 min, 100%. |
| 73 | 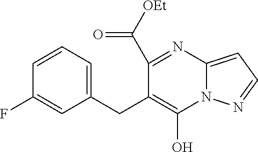 [SMILES: CCOC(=O)c1nc2ccnn2c(O)c1Cc1cccc(F)c1] | Ethyl 6-[(3-fluorophenyl)methyl]-7- hydroxypyrazolo[1,5-a]pyrimidine-5- carboxylate | 85 | O | 34% | Calculated for C₁₆H₁₄FN₃O₃: 315.10192, found 315.10246. HPLC: Rf 5.60 min, 96.8%. |
| 74 | 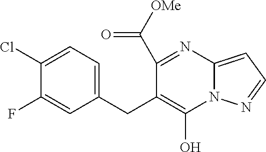 [SMILES: COC(=O)c1nc2ccnn2c(O)c1Cc1ccc(Cl)c(F)c1] | Ethyl 6-[(4-chloro-3-fluorophenyl)methyl]- 7-hydroxy pyrazolo[1,5-a]pyrimidine-5- carboxylate | 86 | O | 1% | Calculated for C₁₆H₁₃ClFN₃O₃: 349.062947, found 349.063487. HPLC: Rf 6.08 min, 96%. |
| 75 | 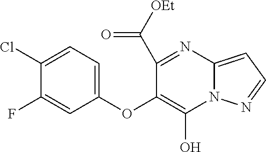 [SMILES: CCOC(=O)c1nc2ccnn2c(O)c1Oc1ccc(Cl)c(F)c1] | Ethyl 6-(4-chloro-3-fluorophenoxy)-7- hydroxypyrazolo[1,5-a]pyrimidine-5- carboxylate | 89 | L | 30% | Calculated for C₁₅H₁₁ClFN₃O₄: 351.042212, found 351.042552. HPLC: Rf 5.59 min, 98.6%. |
| 76 | 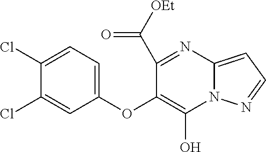 [SMILES: CCOC(=O)c1nc2ccnn2c(O)c1Oc1ccc(Cl)c(Cl)c1] | Ethyl 6-(3,4-dichlorophenoxy)-7-hydroxy- pyrazolo[1,5-a]pyrimidine-5-carboxylate | 90 | L | 41% | Calculated for C₁₅H₁₁Cl₂N₃O₄: 367.012661, found 367.013061. HPLC: Rf 5.69 min, 100%. |
| 77 | 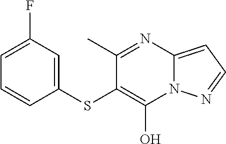 [SMILES: Cc1nc2ccnn2c(O)c1Sc1cccc(F)c1] | 6-[(3-Fluorophenyl)sulfanyl]-5-methyl- pyrazolo[1,5-a]pyrimidin-7-ol | 91 | M | 36% | Calculated for C₃H₁₀FN₃OS: 275.052861, found 275.053491. HPLC: Rf 4.87 min, 100%. |

*Intermediate beta-keto ester/3-aminopyrazole commercially available.

### Example 79

### General Procedure P

### 6-[Ethyl(3-fluorophenyl)amino]-5-methylpyrazolo[1,5-a]pyrimidin-7-ol

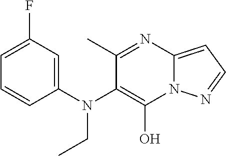 [SMILES: CCN(c1cccc(F)c1)c1c(C)nc2ccnn2c1O]

Intermediate 95 (610 mg, 1.88 mmol) was dissolved in EtOH (7 mL), TiCl₃ (2.50 mL, 30 wt % in 2 M aq HCl, 5.00 mmol) was added and the reaction mixture was stirred for 5 h. A solution of 3-aminopyrazole (311 mg, 3.75 mmol) in EtOH (5 mL) was added and the reaction mixture was heated at reflux for 7 h. The solvents were removed in vacuo and the residue was purified by column chromatography, triturated from Et₂O/MeOH and dried to give the title compound (54.0 mg, 11%) as a white solid. HRMS (ESI⁺) calculated for C₁₅H₁₅FN₄O: 286.122989. found 286.122649. HPLC: Rf 4.60 min, 98.3%.

### Example 80

### 6-[(3-Fluorophenyl)(methyl)amino]-5-methylpyrazolo[1,5-a]pyrimidin-7-ol

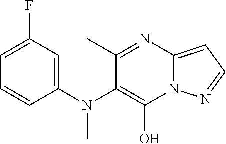 [SMILES: Cc1nc2ccnn2c(O)c1N(C)c1cccc(F)c1]

Intermediate 94 (813 mg, 2.61 mmol) was reacted according to General Procedure P to give the title compound (101 mg, 33%) as a white solid. HRMS (ESI⁺) calculated for C₁₄H₁₃FN₄O: 272.107339. found 272.106659. HPLC: Rf 5.07 min, 98.2%.

### Example 81

### General Procedure Q

### Methyl 6-[(3-chlorophenyl)methyl]-7-hydroxypyrazolo[1,5-a]pyrimidine-5-carboxylate

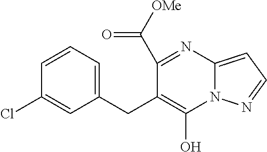 [SMILES: COC(=O)c1nc2ccnn2c(O)c1Cc1cccc(Cl)c1]

Example 70 (400 mg, 1.21 mmol) and sodium hydride (145 mg, 60% dispersion in mineral oil, 3.62 mmol) were dissolved in MeOH (5 mL) and heated using a Biotage microwave (130° C., absorption high) for 30 min. The reaction mixture was acidified with AcOH (0.5 mL) and the precipitate was removed by filtration. The filtrate was concentrated in vacuo and the residue was purified by column chromatography to give the title compound as a white solid (48 mg, 13%). HRMS (ESI⁺) calculated for C₁₅H₁₂ClN₃O₃: 317.056719. found 317.055999. HPLC: Rf 5.45 min, 99%.

### Examples 82-84

Examples 82-84 were prepared similarly to General Procedure Q; see Table 8 below.

### Example 85

### 6-[(3-Chlorophenyl)methyl]-7-hydroxypyrazolo[1,5-a]pyrimidine-5-carboxylic acid

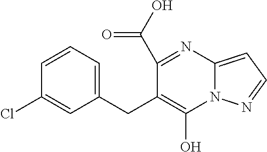 [SMILES: O=C(O)c1nc2ccnn2c(O)c1Cc1cccc(Cl)c1]

Example 70 (400 mg, 1.21 mmol) and sodium hydride (145 mg, 60% dispersion in mineral oil, 3.62 mmol) were dissolved in MeOH (5 mL) and heated using a Biotage microwave (130° C., absorption high) for 30 min. The reaction mixture was acidified with AcOH (0.5 mL) and the precipitate was removed by filtration. The filtrate was concentrated in vacuo and the residue was purified by column chromatography to give the title compound as a white solid (78 mg, 27%). HRMS (ESI⁺) calculated for C₁₄H₁₀ClN₃O₃: 303.041069. found 303.040849. HPLC: Rf 5.45 min, 98.4%.

### Example 86

### Propan-2-yl7-hydroxy-6-{[3-(trifluoromethyl)phenyl]methyl}pyrazolo[1,5-a]pyrimidine-5-carboxylate

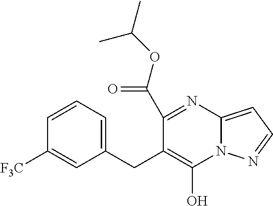 [SMILES: CC(C)OC(=O)c1nc2ccnn2c(O)c1Cc1cccc(C(F)(F)F)c1]

Intermediate 96 (50.0 mg, 0.15 mmol) was dissolved in isopropanol (2.5 mL) and conc sulfuric acid (0.25 mL) was added. The reaction mixture was heated using a Biotage microwave at 100° C. for 1 h. The solvents were removed in vacuo and the residue was purified by HPLC and column chromatography to give the title compound (2.96 mg, 5%) as an off-white solid. HRMS (ESI⁺) calculated for C₁₈H₁₆F₃N₃O₃: 379.114376. found 379.115806. HPLC: Rf 6.28 min, 99.4%.

TABLE 8
Transesterification of R⁶ ethyl esters

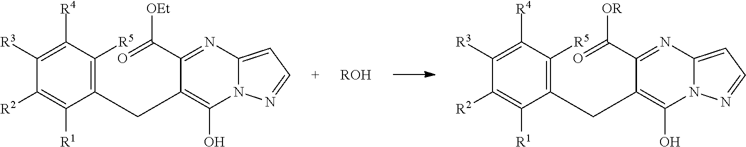

| Ex | Structure | Name | Int | Yield | HRMS (ESI⁺)/HPLC data |
|---|---|---|---|---|---|
| 82 | 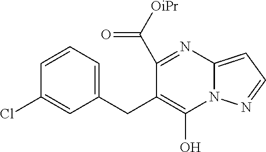 [SMILES: CC(C)OC(=O)c1nc2ccnn2c(O)c1Cc1cccc(Cl)c1] | Propan-2-yl 6-[(3-chlorophenyl)methyl]-7- hydroxypyrazolo[1,5-a]pyrimidine-5- carboxylate | Ex 70 | 47% | Calculated for C₁₇H₁₆ClN₃O₃: 345.088019, found 345.087209. HPLC: Rf 6.17 min, 98.9%. |
| 83 | 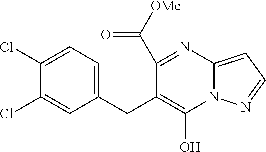 [SMILES: COC(=O)c1nc2ccnn2c(O)c1Cc1ccc(Cl)c(Cl)c1] | Methyl 6-[(3,4-dichlorophenyl)methyl]-7- hydroxypyrazolo[1,5-a]pyrimidine-5- carboxylate | Ex 72 | 40% | Calculated for C₁₅H₁₁Cl₂N₃O₃: 351.017747, found 351.018617. HPLC: Rf 5.90 min, 99.8%. |
| 84 | 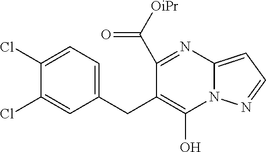 [SMILES: CC(C)OC(=O)c1nc2ccnn2c(O)c1Cc1ccc(Cl)c(Cl)c1] | Propan-2-yl 6-[(3,4-dichlorophenyl)- methyl]-7-hydroxy pyrazolo[1,5-a]- pyrimidine-5-carboxylate | Ex 72 | 39% | Calculated for C₁₇H₁₅Cl₂N₃O₃: 379.049047, found 379.048437. HPLC: Rf 6.47 min, 99.1%. |

### Example 87

### Propan-2-yl6-[(3-chloro-4-fluorophenyl)methyl]-7-hydroxypyrazolo[1,5-a]pyrimidine-5-carboxylate

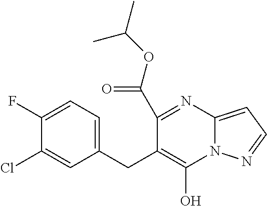 [SMILES: CC(C)OC(=O)c1nc2ccnn2c(O)c1Cc1ccc(F)c(Cl)c1]

The title compound (9.53 mg, 23%) was prepared similarly to Example 86, using Intermediate 99 instead of Intermediate 96, as a cream solid. HRMS (ESI⁺) calculated for C₁₇H₁₅ClFN₃O₃: 363.078597. found 363.079187. HPLC: Rf 6.27 min, 100%.

### Example 88

### Propan-2-yl6-[(4-chloro-3-fluorophenyl)methyl]-7-hydroxypyrazolo[1,5-a]pyrimidine-5-carboxylate

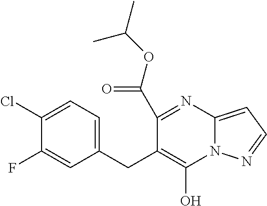 [SMILES: CC(C)OC(=O)c1nc2ccnn2c(O)c1Cc1ccc(Cl)c(F)c1]

The title compound (19.3 mg, 52%) was prepared similarly to Example 86, using Intermediate 100 instead of Intermediate 96. HRMS (ESI⁺) calculated for C₁₇H₁₅ClFN₃O₃: 363.078597. found 363.077937. HPLC: Rf 6.29 min, 100%.

### Example 89

### Methyl 6-(3,4-dichlorophenoxy)-7-hydroxypyrazolo[1,5-a]pyrimidine-5-carboxylate

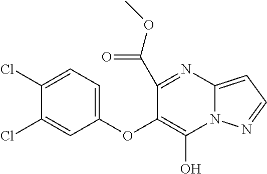 [SMILES: COC(=O)c1nc2ccnn2c(O)c1Oc1ccc(Cl)c(Cl)c1]

Example 76 (250 mg, 0.68 mmol) and sodium hydride (82 mg, 60% dispersion in mineral oil, 2.05 mmol) were dissolved in MeOH (4 mL) and heated using a Biotage microwave at 100° C. for 20 min. The reaction mixture was acidified with AcOH (0.2 mL), concentrated in vacuo and the residue was purified by column chromatography, refluxing in MeOH (50 mL) for 20 min and filtration to give the title compound (28.0 mg, 12%) as a white solid. HRMS (ESI⁺) calculated for C₁₄H₉Cl₂N₃O₄: 352.997011. found 352.997041. HPLC: Rf 5.46 min, 98.7%.

### Example 90

### 6-[(4-Chlorophenyl)methyl]-5-(hydroxymethyl)pyrazolo[1,5-a]pyrimidin-7-ol

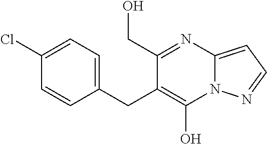 [SMILES: OCc1nc2ccnn2c(O)c1Cc1ccc(Cl)cc1]

Intermediate 102 (380 mg, 1.00 mmol) was suspended in DCM (300 mL) and cooled to −75° C. under argon. A solution of boron trichloride (10.0 mL, 1.00 M in DCM, 10.0 mmol) was added dropwise. The reaction mixture was stirred for 2 h and quenched with 17% ammonia in water (2 mL) and MeOH (50 mL). The reaction mixture was basified to pH 9 and the solvents were removed in vacuo. The residue was partitioned between water (100 mL) and 10% EtOH/EtOAc (800 mL) and the organic layer was washed with 3:1 water:brine (100 mL) and brine (100 mL), dried (MgSO₄) and concentrated in vacuo to give crude product (278 mg). A sample (50.0 mg, 0.17 mmol) was purified by HPLC to give the title compound (9.43 mg, 19%) as a white solid. HRMS (ESI⁺) calculated for C₁₄H₁₂ClN₃O₂: 289.061804. found 289.062944. HPLC: Rf 4.75 min, 100%.

### Example 91

### 6-[(4-Chlorophenyl)methyl]-5-(morpholin-4-ylmethyl)pyrazolo[1,5-a]pyrimidin-7-ol

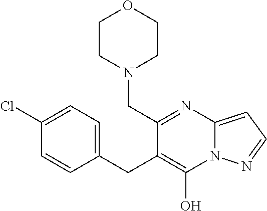 [SMILES: Oc1c(Cc2ccc(Cl)cc2)c(CN2CCOCC2)nc2ccnn12]

Example 90 (60 mg, 0.21 mmol) was suspended in DCM (60 mL) and thionyl chloride (72.0 L, 1.00 mmol) was added dropwise. The reaction mixture was stirred at room temperature for 5 h, thionyl chloride (720 μL, 10.0 mmol) was added and the reaction mixture was stirred for 18 h. Thionyl chloride (1.00 mL, 13.7 mmol) was added and the reaction mixture was stirred for 48 h. The reaction mixture was concentrated in vacuo and the residue partitioned between sat aq NaHCO₃ (35 mL) and EtOAc (25 mL). The aqueous phase was extracted with EtOAc (25 mL) and the combined organic fractions were washed with water (10 mL), brine (25 mL), dried (MgSO₄) and the solvents were removed in vacuo. The residue was dissolved in DMF (2 mL), K₂CO₃ (500 mg) and morpholine (52.0 μL, 0.60 mmol) were added and the reaction mixture was heated at 50° C. for 16 h. The solvents were removed in vacuo and the residue was partitioned between 1 M aq HCl (25 mL) and EtOAc (20 mL). The aqueous layer was acidified to pH 4. The organic layer washed with 1 M aq HCl (15 mL) and the combined aqueous layers were washed with EtOAc (20 mL), basified to pH 8 with NaHCO₃ and extracted with EtOAc (2×20 mL). The combined organic fractions were washed with brine (20 mL), dried (MgSO₄) and the solvents removed in vacuo. The residue was purified by HPLC and dried to give the title compound (25.2 mg, 23%) as a white solid. HRMS (ESI⁺) calculated for C₁₈H₁₉ClN₄O₂: 358.119654. found 358.120454. HPLC: Rf 4.72 min, 100%.

### Example 92

### 6-[(3-Fluorophenyl)methyl]-5-(3-methyl-1,2,4-oxadiazol-5-yl)pyrazolo[1,5-a]pyrimidin-7-ol

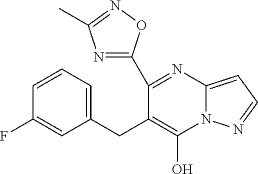 [SMILES: Cc1noc(-c2nc3ccnn3c(O)c2Cc2cccc(F)c2)n1]

Sodium hydride (266 mg, 60% dispersion in mineral oil, 6.64 mmol) was suspended in DMF (20 mL) and acetamide oxime (491 mg, 6.64 mmol) and Example 69 (400 mg, 1.33 mmol) were added. The reaction mixture was heated using a Biotage microwave reactor at 100° C. for 20 min. The solvents were removed in vacuo. The residue was dissolved in EtOH (10 mL) and 1M aq HCl (70 mL) was added. The precipitate was collected by filtration and recrystallised from EtOH to give the title compound (83.0 mg, 19%) as a yellow solid. HRMS (ESI⁺) calculated for C₁₆H₁₂FN₅O₂: 325.097503. found 325.098633. HPLC: Rf 5.42 min, 98.2%.

### Example 93

### 6-[(3-Fluorophenyl)methyl]-5-(2-hydroxypropan-2-yl)pyrazolo[1,5-a]pyrimidin-7-ol

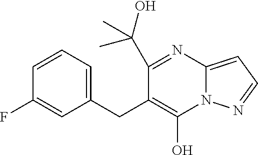 [SMILES: CC(C)(O)c1nc2ccnn2c(O)c1Cc1cccc(F)c1]

Example 69 (100 mg, 0.32 mmol) was dissolved in THF (2 mL), methylmagnesium bromide (3.17 mL, 1 M in THF, 3.17 mmol) was added and the reaction mixture was stirred for 4 h. Methylmagnesium bromide (3.17 mL, 1 M in THF, 3.17 mmol) was added and the reaction mixture was stirred for 16 h. The reaction mixture was quenched with water (1 mL) and the solvents were removed in vacuo. The residue was diluted with water (50 mL) and extracted with EtOAc (2×50 mL). The combined organic fractions were washed with brine (50 mL), dried (MgSO₄) and concentrated in vacuo. The residue was purified by HPLC and column chromatography to give the title compound (5.79 mg, 6%) as an off-white solid. HRMS (ESI⁺) calculated for C₁₆H₁₆FN₃O₂: 301.122655. found 301.122185. HPLC: Rf 4.74 min, 100%.

### Example 94

### General Procedure R

### 1-{6-[(3-Fluorophenyl)methyl]-7-hydroxypyrazolo[1,5-a]pyrimidin-5-yl}ethan-1-one

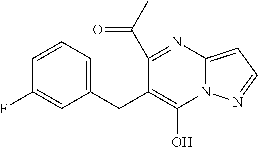 [SMILES: CC(=O)c1nc2ccnn2c(O)c1Cc1cccc(F)c1]

Intermediate 103 (61.0 mg, 0.18 mmol) was dissolved in THF (6 mL), methylmagnesium bromide (1.8 mL, 1 M in THF, 1.80 mmol) was added and the reaction mixture was stirred for 16 h. The solvents were removed in vacuo and the residue diluted with EtOAc (50 mL), washed with 1 M aq citric acid (2×75 mL), dried (MgSO₄) and concentrated in vacuo. The residue was purified by HPLC and recrystallisation from EtOAc to give the title compound (11.2 mg, 22%) as a yellow solid. HRMS (ESI⁺) calculated for C₁₅H₁₂FN₃O₂: 285.091355. found 285.091665. HPLC: Rf 4.94 min, 97.6%.

### Example 95

### 6-[(3-Fluorophenyl)methyl]-5-(1-hydroxyethyl)pyrazolo[1,5-a]pyrimidin-7-ol

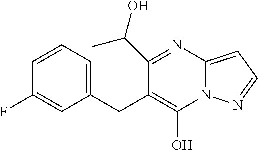 [SMILES: CC(O)c1nc2ccnn2c(O)c1Cc1cccc(F)c1]

Example 94 (42.0 mg, 0.15 mmol) was dissolved in MeOH (2 mL) and sodium borohydride (16.7 mg, 0.44 mmol) was added. The reaction mixture was stirred for 2.5 h, quenched with water (1 mL) and concentrated in vacuo. The residue was purified by HPLC to give the title compound (3.26 mg, 8%) as an off-white solid. HRMS (ESI⁺) calculated for C₁₅H₁₄FN₃O₂: 287.107005. found 287.106765. HPLC: Rf 4.26 min, 99.2%.

### Example 96

### 1-{6-[(3,4-Dichlorophenyl)methyl]-7-hydroxypyrazolo[1,5-a]pyrimidin-5-yl}ethan-1-one

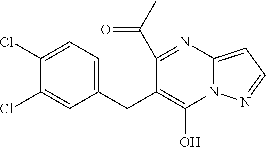 [SMILES: CC(=O)c1nc2ccnn2c(O)c1Cc1ccc(Cl)c(Cl)c1]

Intermediate 104 was reacted according to General Procedure R to give the title compound (10.8 mg, 27%) as a yellow solid. HRMS (ESI⁺) calculated for C₁₅H₁₁Cl₂N₃O₂: 335.022832. found 335.023032. HPLC: Rf 5.82 min, 99.1%.

### Example 97

### 1-{6-[(3,4-Dichlorophenyl)methyl]-7-hydroxypyrazolo[1,5-a]pyrimidin-5-yl}propan-1-one

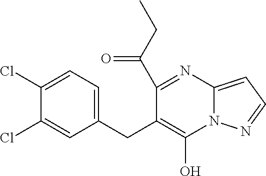 [SMILES: CCC(=O)c1nc2ccnn2c(O)c1Cc1ccc(Cl)c(Cl)c1]

Intermediate 104 and ethylmagnesium bromide were reacted according to General Procedure R to give the title compound (16.6 mg, 36%) as an off-white solid. HRMS (ESI⁺) calculated for C₁₆H₁₃Cl₂N₃O₂: 349.038482. found 349.038512. HPLC: Rf 6.14 min, 99.7%.

### Example 98

### 6-[(4-Chlorophenyl)methyl]-5-[(3-methyl-1,2,4-oxadiazol-5-yl)methyl]pyrazolo[1,5-a]-pyrimidin-7-ol

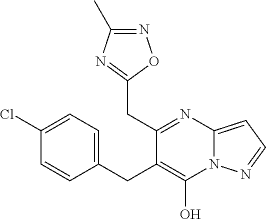 [SMILES: Cc1noc(Cc2nc3ccnn3c(O)c2Cc2ccc(Cl)cc2)n1]

Sodium hydride (116 mg, 60% dispersion in mineral oil, 2.89 mmol) was suspended in DMF (5 mL) and acetamide oxime (214 mg, 2.89 mmol) and Intermediate 106 (200 mg, 0.58 mmol) were added. The reaction mixture was heated using a Biotage microwave at 100° C. for 30 min and the solvents were removed in vacuo. The residue was dissolved in EtOH (30 mL), acidified with 1 M aq HCl (100 mL) and cooled to −22° C. over 60 h. The precipitate was collected by filtration and purified by HPLC to give the title compound (7.50 mg, 4%) as a white solid. HRMS (ESI⁺) calculated for C₁₇H₁₄ClN₅O₂: 355.083602. found 355.083112. HPLC: Rf 5.63 min (gradient 20-100%), 99.9%.

### Example 99

### General Procedure S

### 6-[(4-Chlorophenyl)methyl]-7-hydroxy-N,5-dimethylpyrazolo[1,5-a]pyrimidine-3-carboxamide

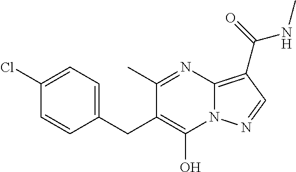 [SMILES: CNC(=O)c1cnn2c(O)c(Cc3ccc(Cl)cc3)c(C)nc12]

Intermediate 115 (15.9 mg, 50.0 μmol), imidazole (10.0 mg, 147 μmol) and DBU (8.00 mg, 52.5 μmol) were dissolved in MeCN (200 μL). T₃P (39.0 μL, 50% solution in EtOAc, 65.6 μmol) was added and the reaction mixture was shaken for 2 h. A solution of methylamine (2.33 mg, 75.0 μmol) in MeCN (200 μL) was added and the reaction mixture was shaken for 7 d. The reaction mixture was purified by column chromatography and dried to give the title compound (3.20 mg, 19%). Analytical HPLC-MS: purity 98%, ES⁺: 331.1 [MH]. HPLC*: Rf 2.08 min, 98%.

### Example 100

### 6-[(4-Chlorophenyl)methyl]-7-hydroxy-N,N,5-trimethylpyrazolo[1,5-a]pyrimidine-3-carboxamide

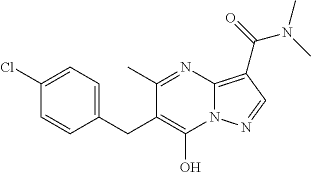 [SMILES: Cc1nc2c(C(=O)N(C)C)cnn2c(O)c1Cc1ccc(Cl)cc1]

Intermediate 115 (300 mg, 0.94 mmol) was dissolved in thionyl chloride (5 mL) and heated at reflux for 2 h. The reaction mixture was concentrated in vacuo and dissolved in THF (5 mL) at 0° C. Dimethylamine (5 mL) was added dropwise and the reaction mixture was stirred at room temperature for 18 h. The solvent was removed in vacuo and the residue was dissolved in CHCl₃ (30 mL) and washed with sat aq NH₄Cl (20 mL), sat. NaHCO₃ (10 mL), brine (2×10 mL), dried (Na₂SO₄) and concentrated in vacuo. The residue was purified by HPLC to give the title compound (180 mg, 55%) as an off-white solid. HRMS (ESI⁺) calculated for C₁₇H₁₇ClN₄O₂: 344.104004. found 344.104784. HPLC: Rf 5.50 min, 98.2%.

### Example 101

### General Procedure T

### 6-[(4-Chlorophenyl)methyl]-7-hydroxy-5-methyl-N-[(2R)-oxolan-2-ylmethyl]-pyrazolo[1,5-a]pyrimidine-3-carboxamide

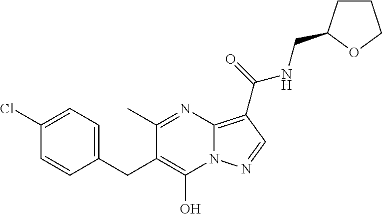 [SMILES: Cc1nc2c(C(=O)NC[C@H]3CCCO3)cnn2c(O)c1Cc1ccc(Cl)cc1]

Intermediate 115 (100 mg, 0.31 mmol) was dissolved in DMF (5 mL) and DIPEA (0.22 mL, 1.26 mmol), HBTU (179 mg, 0.47 mmol), HOBt (106 mg, 0.79 mmol) and (R)-2-tetrahydrofurfuryl amine (95.5 mg, 0.94 mmol) were added. The reaction mixture was stirred at room temperature for 18 h. The solvent was removed in vacuo and the residue was dissolved in EtOAc (30 mL) and washed with sat aq NH₄Cl (20 mL), sat. NaHCO₃ (10 mL), brine (2×10 mL), dried (Na₂SO₄) and concentrated in vacuo. The residue was purified by HPLC to give the title compound (42 mg, 33%) as a white solid. HRMS (ESI⁺) calculated for C₂₀H₂₁ClN₄O₃: 400.130218. found 400.131738. HPLC: Rf 5.60 min, 100%.

### Example 102

### General Procedure U

### 7-Hydroxy-N-(2-methoxyethyl)-5-methyl-6-[(4-methylphenyl)methyl]pyrazolo[1,5-a]pyrimidine-3-carboxamide

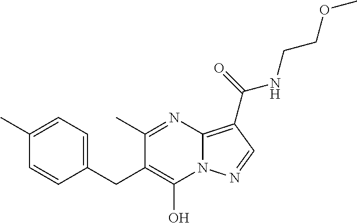

Intermediate 116 (14.9 mg, 50.0 μmol), imidazole (10.0 mg, 147 μmol) and DBN (6.00 mg, 48.3 μmol) were dissolved in MeCN (200 μL). T₃P (39.0 μL, 50% solution in EtOAc, 65.6 μmol) was added and the reaction mixture was shaken for 2 h. A solution of 2-methoxy-ethylamine (5.75 mg, 75.0 μmol) in MeCN (200 uL) was added and the reaction mixture was shaken for 4 d. The reaction mixture was purified by column chromatography to give the title compound (12.7 mg, 72%). HRMS (ESI⁺) for C₁₉H₂₂N₄O₃: 354.169191. found 354.170481. HPLC*: Rf 2.13 min, 100%.

### Example 103

### General Procedure V

### 6-Benzyl-7-hydroxy-5-methyl-N-(2-phenoxyethyl)pyrazolo[1,5-a]pyrimidine-3-carboxamide

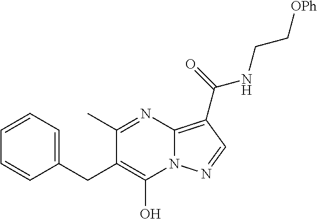 [SMILES: Cc1nc2c(C(=O)NCCOc3ccccc3)cnn2c(O)c1Cc1ccccc1]

Intermediate 117 (14.0 mg, 50.0 μmol) and 1-methylimidazole (8.00 μL, 100 μmol) were dissolved in DMF (200 μL). T₃P (39.0 μL, 50% solution in EtOAc, 65.6 μmol) was added and the reaction mixture was shaken for 1 h. A solution of 2-phenoxy-ethylamine (8.25 mg, 60.0 μmol) in MeCN (200 μL) was added and the reaction mixture was shaken for 7 d. The reaction mixture was purified by column chromatography to give the title compound (4.8 mg, 24%). HRMS (ESI⁺) calculated for C₂₃H₂₂N₄O₃: 402.169191. found 402.170681. HPLC*: Rf 2.35 min, 100%.

### Examples 104-118

Examples 104-118 were prepared similarly to General Procedures S-V, by reacting intermediate carboxylic acids with the required amines; see Table 9 below.

TABLE 9
Esterification of R⁷ carboxylic acids

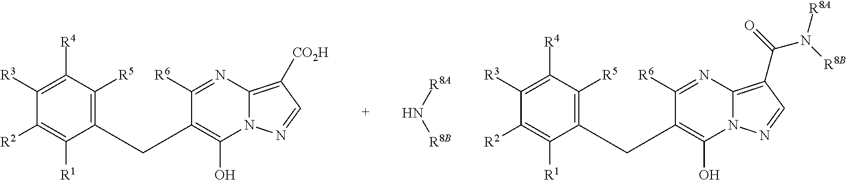

| Ex | Structure | Name | Int | Proc | Yield | HRMS (ESI⁺)/HPLC data |
|---|---|---|---|---|---|---|
| 104 | 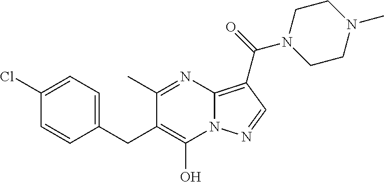 [SMILES: Cc1nc2c(C(=O)N3CCN(C)CC3)cnn2c(O)c1Cc1ccc(Cl)cc1] | 6-[(4-Chlorophenyl)methyl]- 5-methyl-3-[(4- methylpiperazin- 1-yl)carbonyl]pyrazolo [1,5-a]pyrimidin-7-ol | 115 | S | 69% | Calculated for C₂₀H₂₂ClN₅O₂: 399.146203, found 399.146843. HPLC*: Rf 2.08 min, 99%. |
| 105 | 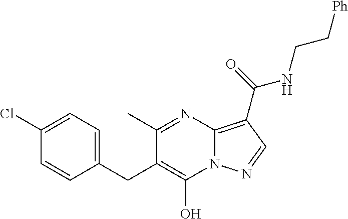 [SMILES: Cc1nc2c(C(=O)NCCc3ccccc3)cnn2c(O)c1Cc1ccc(Cl)cc1] | 6-[(4-Chlorophenyl)methyl]- 7-hydroxy-5-methyl-N- (2-phenylethyl)pyrazolo[1,5- a]pyrimidine-3-carboxamide | 115 | S | 63% | Calculated for C₂₅H₂₁ClN₄O₂: 420.135304, found 420.136604. HPLC*: Rf 2.51 min, 100%. |
| 106 | 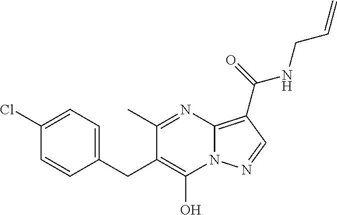 [SMILES: C=CCNC(=O)c1cnn2c(O)c(Cc3ccc(Cl)cc3)c(C)nc12] | 6-[(4-Chlorophenyl)methyl]- 7-hydroxy-5-methyl-N-(prop- 2-en-1-yl)pyrazolo[1,5-a]- pyrimidine-3-carboxamide | 115 | S | 68% | Calculated for C₁₈H₁₇ClN₄O₂: 356.104004, found 356.104874. HPLC*: Rf 2.24 min, 100%. |
| 107 | 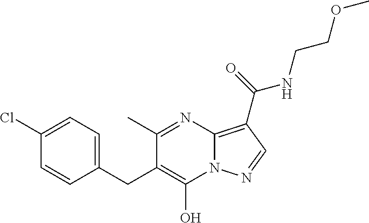 [SMILES: COCCNC(=O)c1cnn2c(O)c(Cc3ccc(Cl)cc3)c(C)nc12] | 6-[(4-Chlorophenyl)methyl]-7- hydroxy-N-(2-methoxyethyl)- 5-methylpyrazolo[1,5-a]- pyrimidine-3-carboxamide | 115 | S | 70% | Calculated for C₁₈H₁₉ClN₄O₃: 374.1146, found 374.1144. HPLC*: Rf 2.13 min, 99%. |
| 108 | 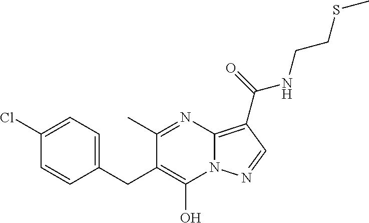 [SMILES: CSCCNC(=O)c1cnn2c(O)c(Cc3ccc(Cl)cc3)c(C)nc12] | 6-[(4-Chlorophenyl)methyl]- 7-hydroxy-5-methyl-N-[2- (methylsulfanyl)ethyl] pyrazolo[1,5-a]pyrimidine-3- carboxamide | 115 | S | 70% | Calculated for C₁₈H₁₉ClN₄O₂S: 390.091724, found 390.092544. HPLC*: Rf 2.29 min, 99%. |
| 109 | 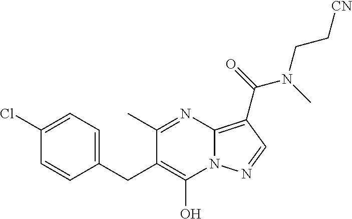 [SMILES: Cc1nc2c(C(=O)N(C)CCC#N)cnn2c(O)c1Cc1ccc(Cl)cc1] | 6-[(4-Chlorophenyl) methyl]-N-(2- cyanoethyl)-7-hydroxy-N,5- dimethylpyrazolo[1,5-a] pyrimidine-3-carboxamide | 115 | S | 66% | Calculated for C₁₉H₁₈ClN₅O₂: 383.114903, found 383.116263. HPLC*: Rf 2.19 min, 100%. |
| 110 | 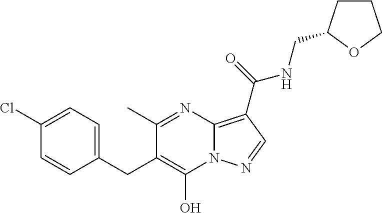 [SMILES: Cc1nc2c(C(=O)NC[C@@H]3CCCO3)cnn2c(O)c1Cc1ccc(Cl)cc1] | 6-[(4-Chlorophenyl) methyl]-7-hydroxy- 5-methyl-N-(oxolan- 2-ylmethyl)pyrazolo-[1,5-a] pyrimidine-3-carboxamide | 115 | T | 18% | Calculated for C₂₀H₂₁ClN₄O₃: 400.130218, found 400.132188. HPLC: Rf 5.60 min, 97.9%. |
| 111 | 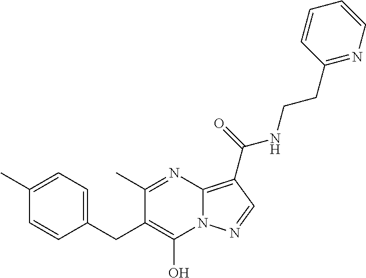 [SMILES: Cc1ccc(Cc2c(C)nc3c(C(=O)NCCc4ccccn4)cnn3c2O)cc1] | 7-Hydroxy-5-methyl-6-[(4- methylphenyl)-methyl]- N-[2-(pyridin-2-yl) ethyl]pyrazolo- [1,5-a]pyrimidine-3- carboxamide | 116 | U | 69% | Calculated for C₂₃H₂₃N₅O₂: 401.185175, found 401.185455. HPLC*: Rf 2.16 min, 100%. |
| 112 | 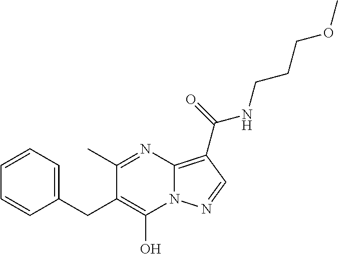 [SMILES: COCCCNC(=O)c1cnn2c(O)c(Cc3ccccc3)c(C)nc12] | 6-Benzyl-7-hydroxy-N-(3- methoxypropyl)-5- methylpyrazolo[1,5-a] pyrimidine-3-carboxamide | 117 | V | 24% | Calculated for C₁₉H₂₂N₄O₃: 354.169191, found 354.170751. HPLC*: Rf 2 min, 100%. |
| 113 | 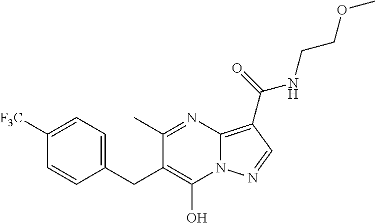 [SMILES: COCCNC(=O)c1cnn2c(O)c(Cc3ccc(C(F)(F)F)cc3)c(C)nc12] | 7-Hydroxy-N-(2- methoxyethyl)-5-methyl-6- {[4-(trifluoromethyl) phenyl]methyl}pyrazolo [1,5-a]pyrimidine-3- carboxamide | 118 | U | 72% | Calculated for C₁₉H₁₉F₃N₄O₃: 408.140925, found 408.142795. HPLC*: Rf 2.24 min, 100%. |
| 114 | 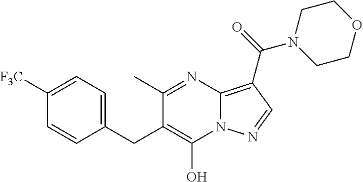 [SMILES: Cc1nc2c(C(=O)N3CCOCC3)cnn2c(O)c1Cc1ccc(C(F)(F)F)cc1] | 5-Methyl-3-(morpholin-4- ylcarbonyl)-6-{[4- (trifluoromethyl)phenyl] ethyl}pyrazolo-[1,5-a] pyrimidin-7-ol | 118 | U | 86% | Calculated for C₂₀H₁₉F₃N₄O₃: 420.140925, found 420.142185. HPLC*: Rf 2.21 min, 100%. |
| 115 | 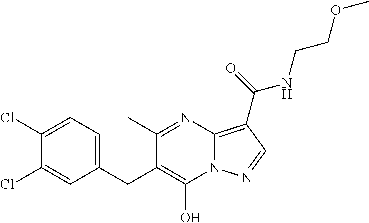 [SMILES: COCCNC(=O)c1cnn2c(O)c(Cc3ccc(Cl)c(Cl)c3)c(C)nc12] | 6-[(3,4-Dichlorophenyl) methyl]-7-hydroxy-N- (2-methoxyethyl)- 5-methylpyrazolo-[1,5-a] pyrimidine-3-carboxamide | 119 | T | 47% | Calculated for C₁₈H₁₈Cl₂N₄O₃: 408.075596, found 408.076886. HPLC: Rf 5.70 min, 99.7%. |
| 116 | 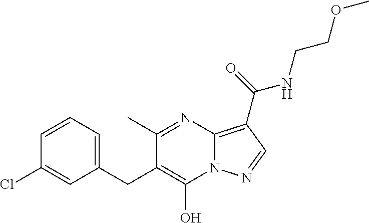 [SMILES: COCCNC(=O)c1cnn2c(O)c(Cc3cccc(Cl)c3)c(C)nc12] | 6-[(3-Chlorophenyl) methyl]-7-hydroxy-N-(2- methoxyethyl)- 5-methylpyrazolo[1,5-a]- pyrimidine-3-carboxamide | 120 | T | 47% | Calculated for C₁₈H₁₉ClN₄O₃: 374.114568, found 374.115188. HPLC: Rf 5.30 min, 96%. |
| 117 | 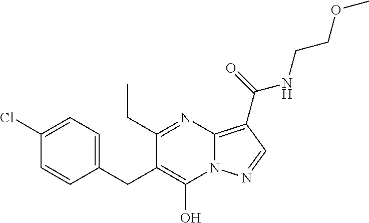 [SMILES: CCc1nc2c(C(=O)NCCOC)cnn2c(O)c1Cc1ccc(Cl)cc1] | 6-[(4-Chlorophenyl)methyl]- 5-ethyl-7-hydroxy-N- (2-methoxyethyl)pyrazolo- [1,5-a]pyrimidine-3- carboxamide | 121 | T | 65% | Calculated for C₁₉H₂₁ClN₄O₃: 388.130218, found 388.131368. HPLC: Rf 5.60 min, 98.8%. |
| 118 | 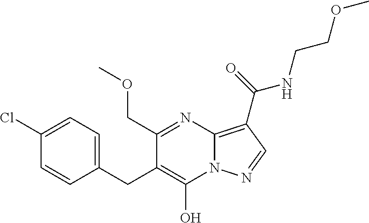 [SMILES: COCCNC(=O)c1cnn2c(O)c(Cc3ccc(Cl)cc3)c(COC)nc12] | 6-[(4-Chlorophenyl)methyl]- 7-hydroxy-N-(2-methoxy- ethyl)5-(methoxymethyl)- pyrazolo[1,5-a]pyrimidine- 3-carboxamide | 122 | T | 11% | Calculated for C₁₉H₂₁ClN₄O₄: 404.125133, found 404.126453. HPLC: Rf 5.70 min, 96.2%. |

### Biological Tests

### CCR2 Functional Calcium Assay

The CCR2 receptor couples through the Gi/Gq signaling pathway and results in activation of calcium mobilization. The functional activity of test compounds was routinely tested by measuring the ability of compounds to antagonize CCR2 activity in a dose dependent manner, in HEK293 EBNA cells transfected with the human CCR2 receptor (hMCP-1 challenge), using a calcium flux Fluorescent Imaging Plate Reader FLIPR assay. Non-transfected HEK293 EBNA cells were used as control of non-specific response.

Briefly, test compounds were dissolved in dimethyl sulfoxide (DMSO) to a concentration of 10 mM and stored in matrix screenmate racks. The required amount of compound was transferred to 96-well compound plates on the day of assay and diluted in assay buffer to the required final concentration; dose-response measurements were assayed by making 1:3 serial dilutions to produce 10 point curves. The compounds were then transferred to 384-well assay plates ready for use. Top concentrations were adjusted depending on the potency of the compounds with a typical concentration range of 30 μM to 0.5 nM being used. The assay buffer used was HBSS buffer supplemented with 20 mM HEPES and 0.1% BSA, pH7.4. The loading/wash buffers were the same as the assay buffer.

Cells were suspended in culture medium at a density of 10000 cells/50 μl (the cell culture media composition was DMEM high glucose supplemented with 10% dialyzed FBS, 250 μg/ml Geneticin, and 400 μg/ml Hygromycin B), transferred to 384-well black/clear Costar plates (Costar #3712) (50 μl/well) and incubated at 37° C., in a 5% CO₂/95% air humidified incubator for 16 h. The cells were washed with assay buffer at 37° C. using the Biotek ELx 405, washing 3 times, leaving 20 μl buffer in the well. 20 μl Fluo-4 (Fluo-4 stock solution (1 mM) was prepared by dissolving one vial of Fluo-4 (50 μg) in 45 μl of pluronic acid (240 mg/ml in DMSO). This stock solution of Fluo-4 was then diluted 250 times with loading buffer to give a Fluo-4 concentration of 4 μM. The dye solution (used within 2 h and kept away from light) was added to each well using a repeating multichannel pipette; the cells were then incubated at 37° C. for 60 min. Following the incubation, cells were washed in assay buffer at 37° C. using the Biotek ELx 405, washing 3 times, leaving 40 μl in each well and incubated for 10 min at 37° C. before use. A combined agonist/antagonist protocol was used. Compound (antagonist) was added to the cell plate using the FLIPR. Basal fluorescence was recorded every second for 10 seconds prior to compound addition (10 μl) and fluorescence recorded every second for 1 minute then every 6 seconds for a further 1 minute. Agonist (MCP-1) was then added using the FLIPR and fluorescence recorded as described above.

The positive control (agonist) was human recombinant MCP-1 which was stored as a stock concentration of 10 μM in distilled water and stored at −20° C. (maximal response: 30 nM; EC₅₀ dose: 3-5 nM). The reference compound (antagonist) was RS102895 which was used as a 10 mM DMSO solution and stored at −20° C. (full inhibition at 2 μM, fKi=84 nM).

FLIPR responses were measured as peak minus basal fluorescence intensity and were expressed as a percentage of EC₅₀ MCP-1 challenge. Curve-fitting and parameter estimation were carried out using GraphPad Prism 4.0 (GraphPad Software Inc., San Diego, Calif.).

The exemplified compounds of the invention were found to be highly potent inhibitors of CCR2 (See Table 10).

### CCR2 Binding Assay ([¹²⁵I]-MCP-1 Displacement)

The binding of test compounds to the CCR2 receptor was evaluated using [¹²⁵I]-MCP-1. Test compounds were shown to displace the radiolabelled ligand in a competitive manner.

Briefly, 25 μL assay buffer (25 mM HEPES, pH 7.4, 5 mM MgCl₂, 1 mM CaCl₂, 0.2% (w/v) protease free BSA, 100 μg/mL bacitracine and 0.1 M NaCl) was placed into total binding wells and 25 μL unlabelled ligand (0.4 μM MCP-1, for determination of non-specific binding) was placed into non-specific binding wells. [¹²⁵I]-MCP-1 (25 μL), human CCR2-HEK293 EBNA membrane preparation (25 μL) and SPA beads (25 μL) were added to all the wells. The wells were incubated for 4 h and counted for 1 min/well in a Perkin Elmer Topcount NXT.

The SPA beads (wheat germ agglutinin (WGA) PEI Type A PVT 0.25 mg/well) were prepared by reconstituting lyophilised bead to 100 mg/mL with de-ionised water and further diluting in assay buffer to give 10 mg/mL. The radioligand ([¹²⁵I]-MCP-1) was prepared by dilution in assay buffer to give 0.32 μCi/mL, ˜17600 dpm/25 μL (specific activity 2000 Ci/mMol). The final assay concentration was 0.04 nM. The human CCR2-HEK293 EBNA cells membranes were prepared as follows: cells were spun down at 1000×g for 3 min at room temperature, washed in PBS and spun down again. The cells were then homogenised with an Ultra Turrax at setting 6 in 5-10 mL ice cold buffer A (EDTA 10 mM, HEPES 10 mM, pH 7.4) for 10 sec. Following dilution with further ice cold buffer A and spinning at 20000×g for 20 min at 4° C., the mixture was re-homogenised in 5-10 mL ice cold buffer B (EDTA 0.1 mM, HEPES 10 mM, pH 7.4) and spun at 20000×g for 20 min at 4° C. The protein was assayed and re-suspended in buffer C (Buffer B+1 tablet/10 mL of Roche protease inhibitor cocktail) at 3 mg/mL. Before use, the membranes were thawed and diluted with assay buffer to give 80 μg/mL (2 μg/well).

Specific binding was determined as the difference between total binding in the absence of antagonist and binding in the presence of excess antagonist (non-specific binding). Data was expressed as a percentage of specific binding and analysed by a 4-parameter logistic equation using GraphPad Prism 4 software (GraphPad, San Diego, Calif., USA) to yield IC₅₀ values. Ki values were calculated from the IC₅₀ values using the correction for radioligand concentration.

Tested exemplified compounds of the invention were found to be highly potent inhibitors of CCR2 (See Table 10).

TABLE 10
CCR2 functional activity and binding data
(A: <10 nM, B: 10-100 nM, C: 100-1000 nM)

| | Functional | Binding |
|---|---|---|
| Example | CCR2 fKi | CCR2 Kᵢ |
| 1 | B | C |
| 2 | C | |
| 3 | C | |
| 4 | A | |
| 5 | B | |
| 6 | B | |
| 7 | C | |
| 8 | C | |
| 9 | B | |
| 10 | C | |
| 11 | C | |
| 12 | A | |
| 13 | A | |
| 14 | A | |
| 15 | A | |
| 16 | A | C |
| 17 | A | |
| 18 | A | |
| 19 | B | |
| 20 | B | |
| 21 | A | B |
| 22 | A | B |
| 23 | A | |
| 24 | A | |
| 25 | A | |
| 26 | A | |
| 27 | A | |
| 28 | A | |
| 29 | A | |
| 30 | A | |
| 31 | A | |
| 32 | A | |
| 33 | A | B |
| 34 | A | |
| 35 | A | |
| 36 | A | |
| 37 | A | A |
| 38 | A | |
| 39 | A | |
| 40 | A | C |
| 41 | A | |
| 42 | A | |
| 43 | A | |
| 44 | A | |
| 45 | A | |
| 46 | A | |
| 47 | A | |
| 48 | A | |
| 49 | A | |
| 50 | A | |
| 51 | B | |
| 52 | B | |
| 53 | A | |
| 54 | B | |
| 55 | A | |
| 56 | A | |
| 57 | C | |
| 58 | B | |
| 59 | C | |
| 60 | B | |
| 61 | C | |
| 62 | A | |
| 63 | B | |
| 64 | A | B |
| 65 | A | C |
| 66 | A | |
| 67 | A | |
| 68 | A | |
| 69 | B | |
| 70 | A | C |
| 71 | B | |
| 72 | A | B |
| 73 | C | |
| 74 | A | |
| 75 | B | |
| 76 | B | |
| 77 | C | |
| 78 | C | |
| 79 | C | |
| 80 | C | |
| 81 | A | |
| 82 | A | |
| 83 | A | |
| 84 | A | |
| 85 | C | |
| 86 | B | |
| 87 | A | |
| 88 | A | |
| 89 | A | |
| 90 | C | |
| 91 | C | |
| 92 | C | |
| 93 | C | |
| 94 | B | |
| 95 | C | |
| 96 | B | |
| 97 | B | |
| 98 | C | |
| 99 | C | |
| 100 | C | |
| 101 | C | |
| 102 | C | |
| 103 | C | |
| 104 | C | |
| 105 | C | |
| 106 | C | |
| 107 | B | |
| 108 | C | |
| 109 | C | |
| 110 | C | |
| 111 | C | |
| 112 | C | |
| 113 | C | |
| 114 | C | |
| 115 | B | |
| 116 | C | |
| 117 | B | |
| 118 | C | |

### In Vivo Efficacy

Diabetic nephropathy is a common manifestation of renal disease and is defined as the progressive development of renal insufficiency in the setting of hyperglycaemia. This sustained hyperglycaemia causes glomerular mesangial expansion through increased synthesis and decreased degradation of extracellular matrix protein, which progressively destroys the glomerular capillaries, eventually leading to proteinuria and renal failure. Animal models for diabetes can be employed for assessing the mechanisms of the disease, screening potential therapies for the treatment of this condition, and evaluation of therapeutic options. Streptozotocin (STZ) is an antibiotic, more specifically an analogue of N-acetylglucosamine which selectively inhibits the activity of beta-cell O-GlcNAcase, an enzyme responsible for the removal of O-GlcNAc from protein. A single intraperitoneal injection of STZ in rats results in selective damage of the insulin producing beta cells in the pancreas causing insulin deficiency and subsequent hyperglycaemia after 48 hours. Over the time course of this procedure which can last from 3 weeks to many months, animals develop modest elevations in albuminuria and serum creatinine and some of the histological lesions associated with diabetic nephropathy. The aim of this study was to determine the efficacy of a test compound CCR2 antagonist (Example 33) in a rat model of STZ induced diabetes.

Male Wistar rats were given daily oral administration of the vehicle (30% w/v hydroxypropyl-beta-cyclodextrin (HPBC)) in saline to groups 1 and 3, or oral administration of Example 33 (8 mg/kg/day) to group 2, from 3 days prior to administration of streptozotocin. On day 0 streptozotocin was injected (50 mg/kg body wt, i.p. dissolved in 20 mM sodium citrate buffer) to rats in groups 1 and 2 (STZ groups). Group 3 rats (sham) were injected with an equivalent volume of 20 mM sodium citrate buffer. On day 43 all animals were culled, the left kidney was removed and cut in a sagittal section. These tissue samples were fixed by immersion in 10% (wt/vol) formaldehyde in phosphate-buffered saline (PBS) (0.01 mol/L, pH 7.4) at room temperature. After dehydration using graded ethanol, the tissue was embedded in Paraplast (Sherwood Medical, Mahwah, N.J., USA) and cut into fine (8 μm) sections and mounted on glass slides. Sections were then deparaffinized with xylene. After deparaffinization, sections were counterstained with hematoxylin and eosin or stained with EDI, and viewed under a light microscope (Zeiss AxioSkop). The measured parameters were 1) monocyte and macrophage infiltration, 2) tubular damage and 3) glomerular damage. A semiquantitative score was assigned to each of the parameters by an observer unaware of the treatment.

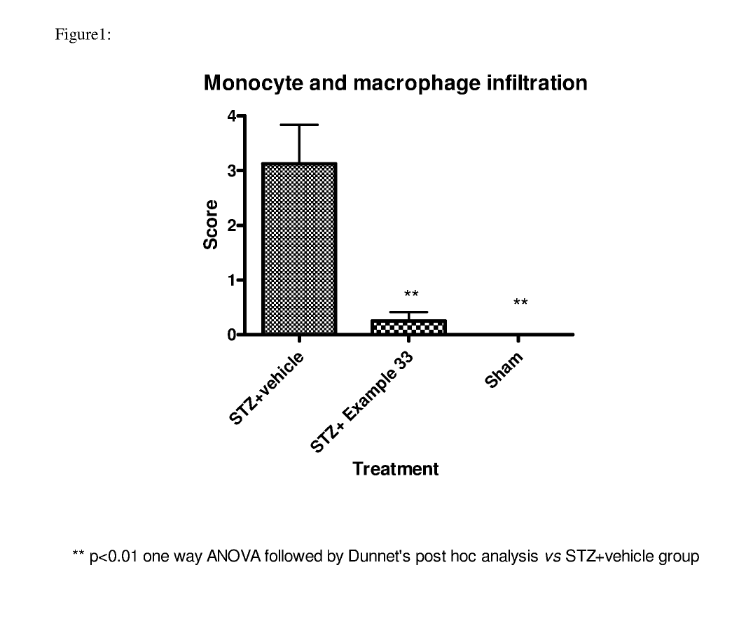

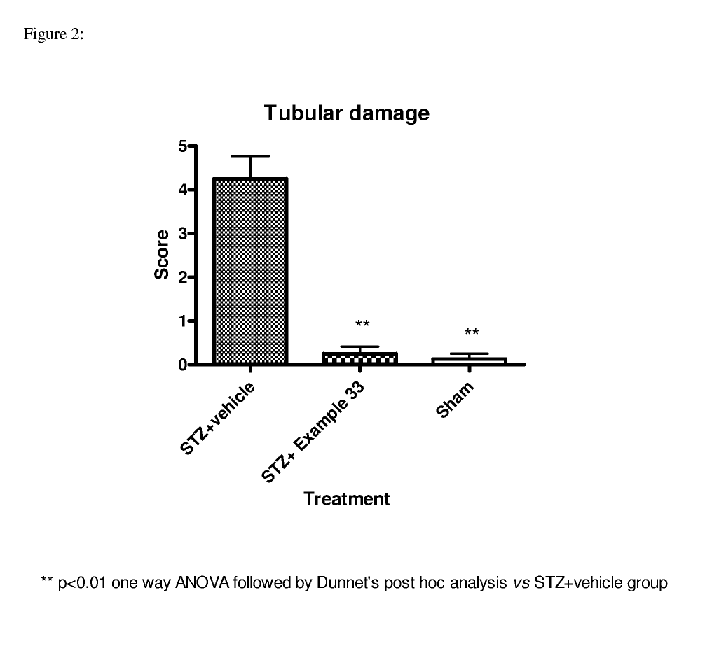

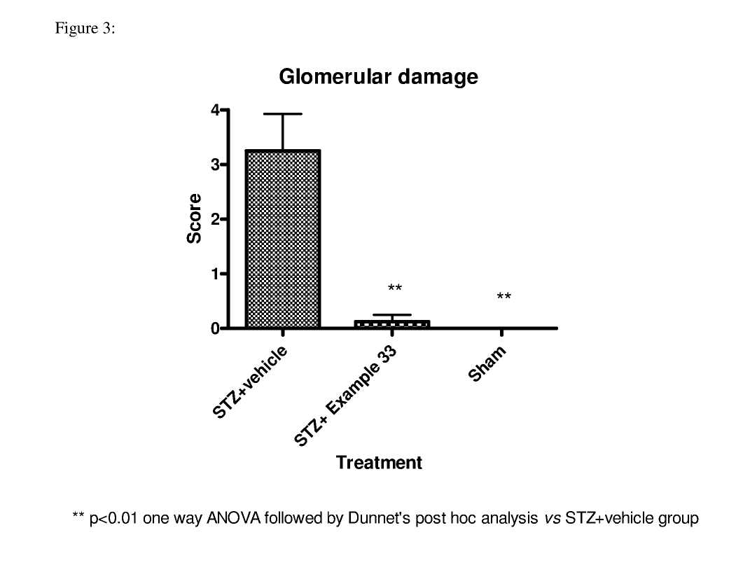

Streptozocin treatment resulted in monocyte and macropage infiltration (FIG. 1), tubular damage (FIG. 2) and glomerular damage (FIG. 3), all statistically significantly higher (p<0.01) than seen in the sham (non-diabetic) group. The administration of the test compound in STZ-treated animals had a statistically significant effect reducing all three parameters (p<0.01) vs the STZ-vehicle treated group. These results illustrate the utility of the test compound, and the compounds of the invention in general, in the treatment of diabetic nephropathy.

1-25. (canceled)

26. A compound of formula (I) 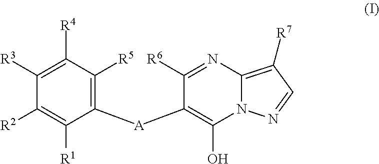
or a pharmaceutically acceptable salt, solvate, hydrate, geometrical isomer, tautomer, optical isomer or N-oxide thereof, wherein:
R¹-R⁵ are each independently selected from hydrogen, halogen, cyano, C₁₋₄-alkyl, C₁₋₄-alkoxy, fluoro-C₁₋₄-alkyl and fluoro-C₁₋₄-alkoxy;
R⁶ is selected from C₁₋₆-alkyl, fluoro-C₁₋₆-alkyl, hydroxy-C₁₋₆-alkyl, C₁₋₄-alkoxy-C₁₋₄-alkyl, C₃₋₅-cycloalkyl, C₁₋₆-alkylcarbonyl, C₁₋₆-alkoxycarbonyl, —CO₂H, heterocyclyl, heterocyclyl-C₁₋₄-alkyl, heteroaryl and heteroaryl-C₁₋₄-alkyl, wherein any heteroaryl residue is optionally substituted with C₁₋₄-alkyl;
R⁷ is selected from hydrogen, halogen, cyano, C₁₋₄-alkyl and —C(O)N(R^{8A})(R^{8B});
A is selected from —CH(R⁹)—, —N(R¹⁰)—, —O— and —S—;
R^{8A} and R^{8B} are each independently selected from hydrogen, C₁₋₄-alkyl, C₂₋₄-alkenyl, cyano-C₁₋₄-alkyl, C₁₋₄-alkoxy-C₁₋₄-alkyl, C₁₋₄-alkylthio-C₁₋₄-alkyl, —C₁₋₄-alkylene-N(R^{11A})(R^{11B}), phenyl-C₁₋₄-alkyl, phenoxy-C₁₋₄-alkyl, heteroaryl-C₁₋₄-alkyl and heterocyclyl-C₁₋₄-alkyl; or
R^{8A} and R^{8B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl;
R⁹ and R¹⁰ are each selected from hydrogen and C₁₋₄-alkyl;
R^{11A} and R^{11B} are each independently selected from hydrogen, C₁₋₄-alkyl and phenyl;
or
R^{11A} and R^{11B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl;
provided that at least one of R¹-R⁵ is selected from halogen, cyano, C₁₋₄-alkyl, C₁₋₄-alkoxy, fluoro-C₁₋₄-alkyl or fluoro-C₁₋₄-alkoxy; and
provided that the compound of formula (I) is not selected from the group consisting of:
6-[(2-Chloro-4-fluorophenyl)methyl]-7-hydroxy-5-methyl-N-(3-pyridinylmethyl)-pyrazolo[1,5-a]pyrimidine-3-carboxamide;
N-(2-Cyanoethyl)-6-[(4-fluorophenyl)methyl]-7-hydroxy-N,5-dimethyl-pyrazolo[1,5-a]pyrimidine-3-carboxamide;
6-[(2-Chloro-4-fluorophenyl)methyl]-7-hydroxy-5-methyl-N-(2-phenylethyl)-pyrazolo[1,5-a]pyrimidine-3-carboxamide;
7-Hydroxy-5-methyl-6-(phenylmethyl)-pyrazolo[1,5-a]pyrimidine-3-carbonitrile;
N-[2-(Butylmethylamino)ethyl]-6-[(2-fluorophenyl)methyl]-7-hydroxy-5-methyl-pyrazolo[1,5-a]pyrimidine-3-carboxamide;
6-[(4-Chlorophenyl)methyl]-7-hydroxy-N,5-dimethyl-N-(phenylmethyl)-pyrazolo[1,5-a]pyrimidine-3-carboxamide;
6-[(3-Chlorophenyl)methyl]-7-hydroxy-5-methyl-N-(2-phenylethyl)-pyrazolo[1,5-a]pyrimidine-3-carboxamide;
N-Butyl-6-[(4-fluorophenyl)methyl]-7-hydroxy-N,5-dimethyl-pyrazolo[1,5-a]pyrimidine-3-carboxamide;
N-Butyl-6-[(2-chlorophenyl)methyl]-7-hydroxy-5-methyl-pyrazolo[1,5-a]pyrimidine-3-carboxamide;
[6-[(4-Chlorophenyl)methyl]-7-hydroxy-5-methylpyrazolo[1,5-a]pyrimidin-3-yl]-1-pyrrolidinyl-methanone;
[6-[(3-Methylphenyl)methyl]-7-hydroxy-5-methylpyrazolo[1,5-a]pyrimidin-3-yl][4-ethyl-1-piperazinyl]-methanone;
6-[(2-Chloro-4-fluorophenyl)methyl]-7-hydroxy-5-methyl-N-[3-(4-morpholinyl)propyl]-pyrazolo[1,5-a]pyrimidine-3-carboxamide;
6-[(4-Chlorophenyl)methyl]-7-hydroxy-N-(2-methoxyethyl)-5-methyl-pyrazolo[1,5-a]pyrimidine-3-carboxamide;
[6-[(2-Fluorophenyl)methyl]-7-hydroxy-5-methylpyrazolo[1,5-a]pyrimidin-3-yl]-1-piperidinyl-methanone;
N-[3-(2-Ethyl-1-piperidinyl)propyl]-6-[(2-fluorophenyl)methyl]-7-hydroxy-5-methyl-pyrazolo[1,5]pyrimidine-3-carboxamide;
6-[(2-Fluorophenyl)methyl]-7-hydroxy-5-methyl-N-(2-phenylethy)-pyrazolo[1,5-a]pyrimidine-3-carboxamide;
[6-[(4-Chorophenyl)methyl]-7-hydroxy-5-methylpyrazolo[1,5-a]pyrimidin-3-yl]-1-piperidinyl-methanone;
6-[(2-Fluorophenyl)methyl]-7-hydroxy-N,5-dimethyl-N-(phenylethyl)-pyrazolo[1,5-a]pyrimidine-3-carboxamide;
N-[2-(Dimethylamino)ethyl]-6-[(2-fluorophenyl)methyl]-7-hydroxy-5-methyl-pyrazolo[1,5-a]pyrimidine-3-carboxamide;
[6-[(2-Chlorophenyl)methyl]-7-hydroxy-5-methylpyrazolo[1,5-a]pyrimidin-3-yl]-1-pyrrolidinyl-methanone;
[6-[(2-Chloro-4-fluorophenyl)methyl]-7-hydroxy-5-methylpyrazolo[1,5-a]pyrimidin-3-yl]-1-pyrrolidinyl-methanone;
[6-[(4-Chlorophenyl)methyl]-7-hydroxy-5-methylpyrazolo[1,5-a]pyrimidin-3-yl]-4-morpholinyl-methanone;
[6-[(4-Methylphenyl)methyl]-7-hydroxy-5-methylpyrazolo[1,5-a]pyrimidin-3-yl]-1-pyrrolidinyl-methanone;
6-[(4-Fluorophenyl)methyl]-7-hydroxy-5-methyl-N-[2-(4-morpholinyl)ethyl]-pyrazolo[1,5-a]pyrimidine-3-carboxamide;
[6-[(2-Chloro-4-fluorophenyl)methyl]-7-hydroxy-5-methylpyrazolo[1,5-a]pyrimidin-3-yl]-4-morpholinyl-methanone;
6-[(2-Chlorophenyl)methyl]-7-hydroxy-5-methyl-N-(phenylmethyl)-pyrazolo[1,5-a]pyrimidine-3-carboxamide;
6-[(2-Fluorophenyl)methyl]-N-(2-furanylmethyl)-7-hydroxy-N,5-dimethyl-pyrazolo[1,5-a]pyrimidine-3-carboxamide;
6-[(4-Chlorophenyl)methyl]-N-[3-(diethylamino)propyl]-7-hydroxy-5-methyl-pyrazolo[1,5-a]pyrimidine-3-carboxamide;
N-[2-(Ethylphenylamino)ethyl]-6-[(2-fluorophenyl)methyl]-7-hydroxy-5-methyl-pyrazolo[1,5-a]pyrimidine-3-carboxamide;
6-[(2-Chlorophenyl)methyl]-7-hydroxy-5-methyl-N-(1-methylpropyl)-pyrazolo[1,5-a]-pyrimidine-3-carboxamide; and
6-[(2-Fluorophenyl)methyl]-7-hydroxy-N,5-dimethyl-pyrazolo[1,5-a]pyrimidine-3-carboxamide.

27. The compound according to claim 26, wherein R⁷ is selected from hydrogen, halogen, cyano, C₁₋₄-alkyl.

28. The compound according to claim 27, wherein R⁷ is H.

29. The compound according to claim 26, wherein R⁶ is selected from C₁₋₄-alkyl, fluoro-C₁₋₄-alkyl, hydroxy-C₁₋₄-alkyl, C₁₋₄-alkoxy-C₁₋₄-alkyl, C₃₋₅-cycloalkyl and C₁₋₄-alkoxycarbonyl.

30. The compound according to claim 26, wherein R⁶ is selected from C₃₋₄-alkyl, C₃₋₄-cycloalkyl and C₁₋₃-alkoxycarbonyl.

31. The compound according to claim 26, wherein R⁶ is ethyl, isopropyl cyclopropyl or cyclobutyl.

32. The compound according to claim 26, wherein A is —CH(R⁹)— or —O—.

33. The compound according to claim 26, wherein A is CH₂.

34. The compound according to claim 26, wherein R¹-R⁵ are each independently selected from hydrogen fluoro, chloro, bromo and CF₃.

35. The compound according to claim 26, wherein R¹ is hydrogen, and R²-R⁵ are each independently selected from fluoro, chloro, bromo and CF₃.

36. The compound according to claim 26, wherein R¹ and R⁵ are hydrogen, and R²-R⁴ are each independently selected from fluoro, chloro, bromo and CF₃.

37. The compound according to claim 26, wherein R¹, R⁴, and R⁵ are hydrogen, and R² and R³ are each independently selected from fluoro, chloro, bromo and CF₃.

38. The compound according to claim 26, wherein R¹, R³, and R⁵ are hydrogen, and R² and R⁴ are each independently selected from fluoro, chloro, bromo and CF₃.

39. The compound according to claim 26, wherein R¹, R², R⁴, and R⁵ are hydrogen, and R³ is selected from fluoro, chloro, bromo and CF₃.

40. The compound according to claim 26, wherein R¹, R³, R⁴, and R⁵ are hydrogen, and R² is selected from fluoro, chloro, bromo and CF₃.

41. The compound according to claim 37, wherein R² and R³ are independently selected from fluoro and CF₃.

42. The compound according to claim 38, wherein R² and R⁴ are independently selected from fluoro and CF₃.

43. A compound of formula (I) for use in therapy: 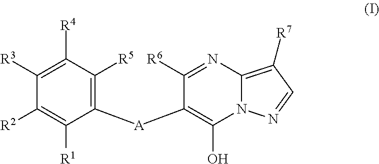
or a pharmaceutically acceptable salt, solvate, hydrate, geometrical isomer, tautomer, optical isomer or N-oxide thereof, wherein:
R¹-R⁵ are each independently selected from hydrogen, halogen, cyano, C₁₋₄-alkyl, C₁₋₄-alkoxy, fluoro-C₁₋₄-alkyl and fluoro-C₁₋₄-alkoxy;
R⁶ is selected from C₁₋₆-alkyl, fluoro-C₁₋₆-alkyl, hydroxy-C₁₋₆-alkyl, C₁₋₄-alkoxy-C₁₋₄-alkyl, C₃₋₅-cycloalkyl, C₁₋₆-alkylcarbonyl, C₁₋₆-alkoxycarbonyl, —CO₂H, heterocyclyl, heterocyclyl-C₁₋₄-alkyl, heteroaryl and heteroaryl-C₁₋₄-alkyl, wherein any heteroaryl residue is optionally substituted with C₁₋₄-alkyl;
R⁷ is selected from hydrogen, halogen, cyano, C₁₋₄-alkyl and —C(O)N(R^{8A})(R^{8B});
A is selected from —CH(R⁹)—, —N(R¹⁰)—, —O— and —S—;
R^{8A} and R^{8B} are each independently selected from hydrogen, C₁₋₄-alkyl, C₂₋₄-alkenyl, cyano-C₁₋₄-alkyl, C₁₋₄-alkoxy-C₁₋₄-alkyl, C₁₋₄-alkylthio-C₁₋₄-alkyl, —C₁₋₄-alkylene-N(R^{11A})(R^{11B}), phenyl-C₁₋₄-alkyl, phenoxy-C₁₋₄-alkyl, heteroaryl-C₁₋₄-alkyl and heterocyclyl-C₁₋₄-alkyl; or
R^{8A} and R^{8B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl;
R⁹ and R¹⁰ are each selected from hydrogen and C₁₋₄-alkyl;
R^{11A} and R^{11B} are each independently selected from hydrogen, C₁₋₄-alkyl and phenyl;
or
R^{11A} and R^{11B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl;
provided that at least one of R¹-R⁵ is selected from halogen, cyano, C₁₋₄-alkyl, C₁₋₄-alkoxy, fluoro-C₁₋₄-alkyl or fluoro-C₁₋₄-alkoxy; and
provided that the compound is not selected from the group consisting of:
N-[2-(Ethylphenylamino)ethyl]-6-[(2-fluorophenyl)methyl]-7-hydroxy-5-methyl-pyrazolo[1,5-a]pyrimidine-3-carboxamide;
6-[(2-Chlorophenyl)methyl]-7-hydroxy-5-methyl-N-(1-methylpropyl)-pyrazolo[1,5-a]-pyrimidine-3-carboxamide; and
6-[(2-Fluorophenyl)methyl]-7-hydroxy-N,5-dimethyl-pyrazolo[1,5-a]pyrimidine-3-carboxamide.

44. A pharmaceutical formulation containing a compound as defined in claim 43 as active ingredient, in combination with a pharmaceutically acceptable diluent or carrier.

45. A compound of formula (I) 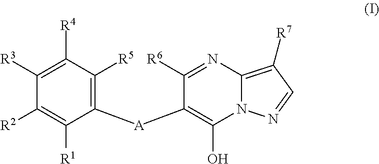
or a pharmaceutically acceptable salt, solvate, hydrate, geometrical isomer, tautomer, optical isomer or N-oxide thereof, for use in the treatment or prevention of, or for the manufacture of a medicament for the treatment or prevention of, medical conditions wherein mediation of the MCP-1/CCR2 pathway is beneficial, in which formula:
R¹-R⁵ are each independently selected from hydrogen, halogen, cyano, C₁₋₄-alkyl, C₁₋₄-alkoxy, fluoro-C₁₋₄-alkyl and fluoro-C₁₋₄-alkoxy;
R⁶ is selected from C₁₋₆-alkyl, fluoro-C₁₋₆-alkyl, hydroxy-C₁₋₆-alkyl, C₁₋₄-alkoxy-C₁₋₄-alkyl, C₃₋₅-cycloalkyl, C₁₋₆-alkylcarbonyl, C₁₋₆-alkoxycarbonyl, —CO₂H, heterocyclyl, heterocyclyl-C₁₋₄-alkyl, heteroaryl and heteroaryl-C₁₋₄-alkyl, wherein any heteroaryl residue is optionally substituted with C₁₋₄-alkyl;
R⁷ is selected from hydrogen, halogen, cyano, C₁₋₄-alkyl and —C(O)N(R^{8A})(R^{8B});
A is selected from —CH(R⁹)—, —N(R¹⁰)—, —O— and —S—;
R^{8A} and R^{8B} are each independently selected from hydrogen, C₁₋₄-alkyl, C₂₋₄-alkenyl, cyano-C₁₋₄-alkyl, C₁₋₄-alkoxy-C₁₋₄-alkyl, C₁₋₄-alkylthio-C₁₋₄-alkyl, —C₁₋₄-alkylene-N(R^{11A})(R^{11B}), phenyl-C₁₋₄-alkyl, phenoxy-C₁₋₄-alkyl, heteroaryl-C₁₋₄-alkyl and heterocyclyl-C₁₋₄-alkyl; or
R^{8A} and R^{8B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl;
R⁹ and R¹⁰ are each selected from hydrogen and C₁₋₄-alkyl;
R^{11A} and R^{11B} are each independently selected from hydrogen, C₁₋₄-alkyl and phenyl;
or
R^{11A} and R^{11B}, together with the nitrogen atom to which they are bound, form a 4- to 6-membered saturated heterocyclic ring which optionally contains an additional heteroatom selected from nitrogen and oxygen, and which ring is optionally substituted with C₁₋₄-alkyl;
provided that at least one of R¹-R⁵ is selected from halogen, cyano, C₁₋₄-alkyl, C₁₋₄-alkoxy, fluoro-C₁₋₄-alkyl or fluoro-C₁₋₄-alkoxy.

46. The compound for use according to claim 45, wherein the medical condition is pain or an inflammatory disease.

47. The compound for use according to claim 45, wherein the medical condition is selected from psoriasis, uveitis, atherosclerosis, rheumatoid arthritis, multiple sclerosis, inflammatory bowel disease, Crohn's disease, nephritis, lupus and lupus nephritis, organ allograft rejection, fibroid lung, renal insufficiency, IgA nephropathy, renal fibrosis, diabetes and diabetic complications, diabetic nephropathy, diabetic retinopathy, diabetic retinitis, diabetic microangiopathy, obesity, diabetic and other forms of neuropathy, neuropathic pain (including that associated with diabetes), chronic pain, giant cell arteritis and other vasculitic inflammatory diseases, tuberculosis, sarcoidosis, invasive staphylococcia, inflammation after cataract surgery, allergic rhinitis, allergic conjunctivitis, chronic urticaria, chronic obstructive pulmonary disease (COPD), allergic asthma. HIV associated dementia, periodontal diseases, periodontitis, gingivitis, gum disease, diastolic cardiomyopathies, cardiac infarction, myocarditis, chronic heart failure, angiostenosis, restenosis, reperfusion disorders, glomerulonephritis (including but not restricted to focal and segmental glomerulosclerosis, IgA glomerulonephritis. IgM glomerulonephritis, membranoproliferative glomerulonephritis, membranous glomerulonephritis, minimal change nephropathy, vasculitis (including microscopic polyarteritis, Wegener's granulomatosis, Henoch Schonlein purpura and polyarteritis nodosa)), solid tumors and cancers, chronic lymphocytic leukemia, chronic myelocytic leukemia, multiple myeloma, malignant myeloma, Hodgkin's disease, and carcinomas of the bladder, breast, cervix, colon, rectum, lung, prostate and stomach.

48. A compound of formula (I) as defined in claim 45 or a pharmaceutically acceptable salt, solvate, hydrate, geometrical isomer, tautomer, optical isomer or N-oxide thereof, for use in the inhibition of, or for use in the manufacture of a medicament for the inhibition of, the spread of metastatic tumour cells from the site of a primary tumour.
